(19) United States
(12) Patent Application Publication
(10) Pub. No.: US 2022/0313129 A1
(43) Pub. Date: Oct. 6, 2022

(54) DEVICES AND METHODS FOR RECEIVING FLUIDS

(71) Applicant: YourBio Health, Inc., Medford, MA (US)

(72) Inventors: Vincent J. Barone, Medford, MA (US); Ping Gong, Belmont, MA (US); Timothy M. Blicharz, Charlestown, MA (US); Hershel S.G. Macaulay, Palo Alto, CA (US); Jessica A. Wakefield, Boston, MA (US)

(73) Assignee: YourBio Health, Inc., Medford, MA (US)

(21) Appl. No.: 17/607,944

(22) PCT Filed: May 1, 2020
(86) PCT No.: PCT/US2020/031202

§ 371 Date: Nov. 1, 2021

Related U.S. Application Data
(60) Provisional application No. 62/842,303, filed on May 2, 2019.
(60) Provisional application No. 62/880,137, filed on Jul. 30, 2019.
(60) Provisional application No. 62/942,540, filed on Dec. 2, 2019.
(60) Provisional application No. 62/948,788, filed on Dec. 16, 2019.
(60) Provisional application No. 62/959,868, filed on Jan. 10, 2020.

(51) Int. Cl. A61B 5/15; A61B 5/151

(52) U.S. Cl. CPC A61B 5/150099; A61B 5/150022; A61B 5/150068; A61B 5/150412; A61B 5/150984; A61B 5/151; A61B 2010/008

(57) ABSTRACT

The present disclosure generally relates to receiving bodily fluid through a device opening. In one aspect, the device includes an interface that facilitates piercing of skin and/or withdrawal of fluid from the skin. The skin may be subjected to vacuum from a vacuum source.

### RELATED APPLICATIONS

This application claims the benefit of U.S. Provisional Patent Application Ser. No. 62/842,303, filed May 2, 2019; U.S. Provisional Patent Application Ser. No. 62/880,137, filed Jul. 30, 2019; U.S. Provisional Patent Application Ser. No. 62/942,540, filed Dec. 2, 2019; U.S. Provisional Patent Application Ser. No. 62/948,788, filed Dec. 16, 2019; and U.S. Provisional Patent Application Ser. No. 62/959,868, filed Jan. 10, 2020. Each of the above is incorporated herein by reference.

### FIELD

The present disclosure generally relates to systems and methods for receiving fluids or other materials, such as blood or interstitial fluid, from subjects, e.g., from the skin and/or beneath the skin.

### BACKGROUND

Phlebotomy or venipuncture is the process of obtaining intravenous access for the purpose of intravenous therapy or obtaining a sample of venous blood. This process is typically practiced by medical practitioners, including paramedics, phlebotomists, doctors, nurses, and the like. Substantial equipment is needed to obtain blood from a subject, including the use of evacuated (vacuum) tubes, e.g., such as the Vacutainer™ (Becton, Dickinson and company) and Vacuette™ (Greiner Bio-One GmBH) systems. Other equipment includes hypodermic needles, syringes, and the like. However, such procedures are complicated and require sophisticated training of practitioners, and often cannot be done in non-medical settings. Accordingly, improvements in methods of obtaining blood or other fluids from or through the skin are still needed.

Sampling capillary blood by fingerstick requires less training than venipuncture and can be self-administered. Disadvantages to fingerstick sampling are that it is painful, and it can be difficult to reliably obtain a blood sample of sufficient volume and quality for testing. The practice of lancing other sites such as the arm, thigh, or palm has been used to alleviate the pain associated with a fingerstick, but the lower capillary densities in these regions make it difficult to obtain an adequate sample volume for testing.

### SUMMARY

In some embodiments, the present disclosure generally relates to devices and methods for receiving fluids from a subject, such as blood. The subject matter of the present disclosure involves, in some cases, interrelated products, alternative solutions to a particular problem, and/or a plurality of different uses of one or more systems and/or articles.

In one aspect of the disclosure, a device for receiving fluid from a subject is provided. The device includes a device actuator, one or more needles or flow activators configured to cause fluid to be released from the subject, a vacuum source, a support having a sidewall, and an interface configured to contact the subject's skin, the interface defining an opening through which fluid is received from the subject. At least a portion of the interface is moveable relative to the sidewall of the support in some cases.

In another aspect of the disclosure, a device for receiving fluid from a subject is provided. The device includes a housing including an inlet sidewall defining an opening to receive fluid into the housing, a device actuator, one or more needles or flow activators configured to cause fluid to be released from the subject, and an interface configured to contact the subject's skin. The interface in some cases includes a distal surface configured to contact the subject's skin, and the inlet sidewall includes a distal end, wherein a surface area of the distal surface of the inlet sidewall is larger than a surface area of the distal end of the inlet sidewall in certain embodiments.

In another aspect of the disclosure, a device for receiving fluid from a subject is provided. The device includes a device actuator, one or more needles or flow activators configured to cause fluid to be released from the subject, a vacuum source, and an interface configured to contact the subject's skin. The interface defines an opening through which fluid is received from the subject. The interface has a sidewall comprising a funnel shape.

In another aspect of the disclosure, a device for receiving fluid from a subject is provided. The device includes a device actuator, one or more needles or flow activators configured to cause fluid to be released from the subject, a vacuum source comprising a flexible dome made of a first material, and a shell made of a second material having a higher Young's modulus than that of the first material. The device actuator is moveable relative to the shell. Movement of the device actuator relative to the shell causes compression of the flexible dome.

In another aspect of the disclosure, a device for receiving fluid from a subject is provided. In one set of embodiments, the device comprises one or more flow activators configured to cause fluid to be released from the subject upon insertion into the skin of the subject; a vacuum source able to apply reduced pressure to the skin to withdraw the fluid released from the subject; and an interface configured to contact the skin of the subject, the interface defining an opening through which the fluid is received from the subject into the device, wherein the interface initially contacts the skin at a first contact region, and after the reduced pressure is applied to the skin, the interface contacts the skin at a second contact region. In some cases, the second contact region circumscribes the first contact region. In certain embodiments, when vacuum is applied, the skin is drawn into the interface region, thereby causing the interface to contact the skin at the second contact region.

In another aspect of the disclosure, a device for receiving fluid from a subject is provided. In one set of embodiments, the device comprises one or more flow activators configured to cause fluid to be released from the subject upon insertion into the skin of the subject; a vacuum source able to apply reduced pressure to the skin to withdraw the fluid released from the subject; and an interface configured to contact the skin of the subject at a contact region, the interface defining an opening through which the fluid is received from the subject into the device, wherein the interface is configured to diffuse force substantially evenly at the contact region. In some cases, the force applied to the skin via the interface at any location varies no more than +/−20% from the average force applied to the skin.

In another aspect of the disclosure, a device for receiving fluid from a subject is provided. In one set of embodiments, the device comprises one or more flow activators configured to cause fluid to be released from the subject upon insertion into the skin of the subject; a vacuum source able to apply reduced pressure to the skin to withdraw the fluid released from the subject; and an interface configured to contact the skin of the subject, the interface defining an opening through which the fluid is received from the subject into the device, wherein the interface has a Young's Modulus of less than 1 GPa. In some cases, the Young's Modulus may be less than 30 GPa, less than 20 GPa, less than 10 GPa, less than 5 GPa, less than 3 GPa, less than 2 GPa, less than 1 GPa, less than 500 MPa, less than 300 MPa, less than 200 MPa, less than 100 MPa, less than 50 MPa, less than 30 MPa, less than 20 MPa, less than 10 MPa, less than 5 MPa, etc.

In another aspect of the disclosure, a device for receiving fluid from a subject is provided. In one set of embodiments, the device comprises one or more flow activators configured to cause fluid to be released from the subject upon insertion into the skin of the subject; a vacuum source able to apply reduced pressure to the skin to withdraw the fluid released from the subject; and an interface configured to contact the skin of the subject, the interface defining an opening through which the fluid is received from the subject into the device, wherein the interface defines a surface that slopes inwardly towards the opening. In some cases, the slope exceeds at least 3°, at least 5°, at least 7°, or at least 10° at at least one location within the interface.

In another aspect, the present disclosure encompasses methods of making one or more of the embodiments described herein, for example, a device for receiving fluid. In still another aspect, the present disclosure encompasses methods of using one or more of the embodiments described herein, for example, a device for receiving fluid.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments that incorporate one or more aspects of the disclosure will be described by way of example with reference to the accompanying figures, which are schematic and are not necessarily intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1A is a side view of a support and interface of a fluid receiving device in accordance with aspects of the disclosure;

FIG. 1B is a cross-section view of the support and interface of FIG. 1A;

FIG. 2 is a cross-section view of the support and interface of FIG. 1A integrated with a fluid receiving module to form a fluid receiving device;

FIG. 3A is the support and interface of FIG. 1A at atmospheric conditions;

FIG. 3B is the support and interface of FIG. 1B under vacuum conditions;

FIG. 4A is a side view of one embodiment of a support and interface of a fluid receiving device;

FIG. 4B is a cross-section view of the support and interface of FIG. 4A;

FIG. 5A is a bottom perspective view of the support and interface of FIG. 4A;

FIG. 5B is a partial cutaway perspective view of the support and interface of FIG. 4A;

FIG. 6 is a perspective view of the support and interface of FIG. 4A integrated with a fluid receiving module to form a fluid receiving device;

FIG. 7 is a side view of the fluid receiving device of FIG. 6;

FIG. 8 is a bottom view of the fluid receiving device of FIG. 6;

FIG. 9 is a cross-section view of the fluid receiving device of FIG. 6 along line 9-9 of FIG. 8;

FIG. 10A is a side view of one embodiment of a support and interface of a fluid receiving device;

FIG. 10B is a cross-section view of the support and interface of FIG. 10A;

FIG. 11A is a side view of one embodiment of a support and interface of a fluid receiving device;

FIG. 11B is a cross-section view of the support and interface of FIG. 11A;

FIG. 12 is a cross-section view of one embodiment of a support and interface of a fluid receiving device;

FIG. 13A is a side view of one embodiment of a support and interface of a fluid receiving device;

FIG. 13B is a cross-section view of the support and interface of FIG. 13A;

FIG. 14A is a side view of one embodiment of a support and interface of a fluid receiving device;

FIG. 14B is a cross-section view of the support and interface of FIG. 14A;

FIG. 15A is a side view of one embodiment of a fluid receiving device having the interface of the FIG. 14A embodiment;

FIG. 15B is a cross-section view of the fluid receiving device of FIG. 15A;

FIG. 16A is a side view of one embodiment of a fluid receiving device having an interface; and

FIG. 16B is a cross-section view of the fluid receiving device of FIG. 16A.

FIG. 17A is a perspective view of a support and interface of one embodiment integrated with a fluid receiving module to form a fluid receiving device;

FIG. 17B is a side view of the fluid receiving device of FIG. 17A;

FIG. 17C is a front view of the fluid receiving device of FIG. 17A;

FIG. 17D is a cross-section view of the fluid receiving device of FIG. 17A taken along line 17D-17D in FIG. 17C;

FIG. 17E is a partial cutaway view of the fluid receiving device of FIG. 17A taken along line 17E-17E in FIG. 17C;

FIG. 18 is a perspective view of one embodiment of a fluid receiving device having a device actuator and a shell according to one aspect;

FIG. 19 is a front, perspective, partial cutaway view of the fluid receiving device of FIG. 18, where a portion of the shell is hidden from view;

FIG. 20 is a rear, perspective, partial cutaway view of the fluid receiving device of FIG. 18, where a portion of the shell is hidden from view;

FIG. 21 is the fluid receiving device of FIG. 18 with the device actuator in a deployed position and the flexible dome in a compressed configuration;

FIG. 22 is a rear, perspective, partial cutaway view of the fluid receiving device of FIG. 21 with the device actuator in the deployed position and the flexible dome in the compressed configuration;

FIG. 23 is a perspective, partial cutaway view of the fluid receiving device of FIG. 18, where the piercing assembly, a portion of the shell, and a portion of the flexible dome are hidden from view;

FIG. 24 is a perspective, partial cutaway view of the fluid receiving device of FIG. 23 with the device actuator in a deployed position and the flexible dome in a compressed configuration;

FIG. 25 is a cross-section view of the fluid receiving device of FIG. 18 showing the interaction between the device actuator, shell and flexible dome;

FIG. 26 is a perspective view of a shell of a fluid receiving device;

FIG. 27 is a top view of the shell of FIG. 26;

FIG. 28 is a perspective view of a device actuator;

FIG. 29 is a bottom view of the device actuator of FIG. 28;

FIG. 30 is a partial cutaway view of a fluid receiving device showing the interaction between the device actuator and the shell;

FIG. 31 is a perspective view of another embodiment of a device actuator having a ratchet;

FIG. 32 is a bottom view of the device actuator of FIG. 31;

FIG. 33 is a side view of the device actuator of FIG. 31;

FIGS. 34A-34G depict a sequence of interactions between the ratchet on the device actuator and a pawl on the shell;

FIG. 35 is a perspective view of a piercing assembly of one embodiment;

FIG. 36 is an exploded view of the piercing assembly of FIG. 35;

FIG. 37 is a top view of the piercing assembly of FIG. 35;

FIG. 38 is a cross-section view of the piercing assembly of FIG. 35 along line 38-38 of FIG. 37;

FIG. 39 is a top view of the piercing assembly of FIG. 35;

FIG. 40 depicts a cross-section view of the piercing assembly of FIG. 35 along line 40-40 of FIG. 39 and a detail view of a notch in a guide housing of the piercing assembly;

FIG. 41 is a perspective view of the piercing assembly of FIG. 35 and a detail view of the notch of the guide housing;

FIG. 42 is a perspective view of a guide housing of the piercing assembly of FIG. 35;

FIG. 43 is a front view of the guide housing of FIG. 42;

FIG. 44 is a top view of the guide housing of FIG. 42;

FIG. 45 is a perspective view of a support ring of the piercing assembly of FIG. 35;

FIG. 46 is another perspective view of the support ring of FIG. 45;

FIG. 47 is a top view of the support ring of FIG. 45;

FIG. 48 is a perspective view of a vacuum source in the form of a flexible dome;

FIG. 49 is a side view of the flexible dome of FIG. 48;

FIG. 50 is a cross-sectional view of the flexible dome of FIG. 48 taken along line 50-50 of FIG. 49;

FIG. 51 is a cross-sectional view of an alternative shape for a flexible dome;

FIG. 52 is a cross-sectional view of an alternative shape for a flexible dome;

FIG. 53 is a schematic illustration of a distance-based latch release;

FIG. 54 is a schematic illustration of a force-based latch release;

FIG. 55 is a schematic illustration of a deployment actuator and retraction actuator arranged as springs in series;

FIG. 56 is a schematic illustration of a deployment actuator and retraction actuator arranged as springs in parallel;

FIG. 57 is a perspective view of one embodiment of a fluid receiving device having a device actuator according to one aspect;

FIG. 58 is a partial cutaway view of the fluid receiving device of FIG. 57, where a portion of the housing is hidden from view;

FIG. 59 is a rear perspective view of another partial cutaway view of the fluid receiving device of FIG. 57;

FIG. 60 is an exploded view of the fluid receiving device of FIG. 57 according to one embodiment;

FIG. 61 is an exploded view of a piercing assembly including a spring and latch assembly, needles, and a guide housing, according to one embodiment;

FIG. 62 is the piercing assembly of FIG. 61 in an assembled state;

FIG. 63 is a partial cutaway view of the piercing assembly of FIG. 62;

FIG. 64 is another partial cutaway view of the piercing assembly of FIG. 62;

FIG. 65 is a cross-sectional view of a spring and latch assembly of the piercing assembly of FIG. 61;

FIG. 66 is a top view of the guide housing of FIG. 61;

FIG. 67 is a front view of the guide housing of FIG. 66;

FIG. 68 is a partial cutaway view of the guide housing of FIG. 66;

FIG. 69 is a perspective view of the housing of the fluid receiving device of FIG. 57;

FIG. 70 is a top view of the housing of FIG. 69;

FIG. 71 is an exploded view of an alternative embodiment of a piercing assembly;

FIG. 72 is a further exploded view of the piercing assembly of FIG. 71;

FIG. 73 is the piercing assembly of FIG. 71 in an assembled state;

FIG. 74 is a partial cutaway view of the piercing assembly of FIG. 71;

FIG. 75 is another partial cutaway view of the piercing assembly of FIG. 71;

FIG. 76 is a top view of the guide housing of FIG. 71;

FIG. 77 is a partial cutaway view of the guide housing of FIG. 71;

FIGS. 78A-78M are illustrative embodiments of cross-sections of different support shapes; and

FIGS. 79A-79F are illustrative embodiments of different interface arrangements.

### DETAILED DESCRIPTION

Aspects of the disclosure are not limited in application to the details of construction and the arrangement of components set forth in the following description or illustrated in the drawings. For example, illustrative embodiments relating to piercing skin and receiving blood released from the pierced skin are discussed below, but aspects of the disclosure are not limited to use with devices that pierce skin and/or receive blood. Other embodiments may be employed, such as devices that receive other bodily fluids without piercing, and aspects of the disclosure may be practiced or be carried out in various ways. Also, the phraseology and terminology used herein is for the purpose of description and should not be regarded as limiting.

It is appreciated that, with some fluid receiving devices, when obtaining fluid from pierced skin via vacuum, the device is pressed down onto the skin, which imparts a force onto the skin. It is recognized that the force imparted to the skin may have an effect on the quality and quantity of fluid that is withdrawn from the skin. For example, in the case of blood, it is recognized that, if the device pinches, compresses and/or stretches the skin too much, fluid may be impeded from being withdrawn from the skin, e.g. due to blood vessels deforming and collapsing in reaction to the forces imparted to the skin.

According to one aspect, in some embodiments, the device has an interface that helps distribute force to the skin to avoid high concentrations of force on the skin. For example, in some cases, the interface may be configured to diffuse force substantially evenly at the contact region. For instance, the force applied to the skin via the interface at any location of the skin may vary by no more than +/−50%, no more than +/−40%, no more than +/−30%, no more than +/−20%, no more than +/−10%, no more than +/−5%, no more than +/−3%, no more than +/−2%, or no more than +/−2% from the average force applied to the skin

It is also recognized that, in some embodiments, piercing into and withdrawing fluid from skin that has bulged into certain shapes in reaction to application of vacuum may give rise to increased fluid volume and/or quality. Improved quality may include factors such as avoiding damage to red blood cells and release of cell contents such as hemoglobin or potassium, or decreasing activation of coagulation. In some cases, a skin bulge may help piercing needles to penetrate into the skin at the full insertion depth of the needles. The skin bulge may keep the skin taut, and it may be easier for needles to penetrate into taut skin.

It is also recognized that, in some cases, bulging of skin may induce vasodilation and increase blood flow. For instance, a skin bulge may result in bulge tissue deformation that mechanically dilates blood vessels that reside within the tissue. This vasodilation could be amplified by the body's physiological response to the forces applied to the skin.

According to one aspect, in some embodiments, the device has an interface that permits skin movement under the device to permit skin recruitment into a device opening and to promote desirable skin bulging to facilitate piercing of skin and subsequent withdrawal of fluid from the skin. In some cases, skin movement might happen after piercing of skin, e.g., movement may occur due to vacuum that is generated after skin is pierced.

According to another aspect, in some embodiments, the device interface helps to maintain a seal with the skin during skin bulging and/or other movement.

Embodiments described herein relate to a fluid receiving device having an interface for facilitating fluid withdrawal from a subject. In some embodiments, the interface may be integrated with a fluid receiving module that may serve to promote withdrawal of fluid from a subject. The fluid receiving module may include one or more of the following components: a vacuum source, a fluid storage chamber, and a flow activator. In some embodiments, the fluid receiving device is arranged to pierce the skin of a subject, subject the pierced skin to vacuum to draw fluid out of the skin, and collect the fluid inside the device. The device may be arranged to deploy a plurality of microneedles into the skin. The device may be positioned on any suitable location on the subject, for example, on the arm or leg, on the back, on the abdomen, etc.

The subject is usually human, although non-human subjects may be used in certain instances, for instance, other mammals such as a dog, a cat, a horse, a rabbit, a cow, a pig, a sheep, a goat, a rat (e.g., Rattus Norvegicus), a mouse (e.g., Mus musculus), a guinea pig, a hamster, a primate (e.g., a monkey, a chimpanzee, a baboon, an ape, a gorilla, etc.), or the like.

The device may be actuated by the subject, and/or by another person (e.g., a health care provider, such as a doctor), or the device itself may be self-actuating, e.g., upon application to the skin of a subject.

In one set of embodiments, the vacuum source is a pressure regulator that creates a pressure differential (such as a vacuum). The pressure regulator may be a pressure controller component or system able to create a pressure differential between two or more locations. The pressure differential should be at least sufficient to urge the movement of fluid or other material in accordance with various embodiments as discussed herein, and the absolute pressures at the two or more locations are not important so long as their differential is appropriate, and their absolute values are reasonable for the purposes discussed herein. For example, the pressure regulator may produce a pressure higher than atmospheric pressure in one location, relative to a lower pressure at another location (atmospheric pressure or some other pressure), where the differential between the pressures is sufficient to cause fluid transport. In another example, the regulator or controller will involve a pressure lower than atmospheric pressure (a vacuum) in one location, and a higher pressure at another location(s) (atmospheric pressure or a different pressure) where the differential between the pressures is sufficient to transport fluid. Wherever “vacuum” or “pressure” is used herein, in association with a pressure regulator or pressure differential, it should be understood that the opposite can be implemented as well, as would be understood by those of ordinary skill in the art, e.g., a vacuum chamber can be replaced in many instances with a pressure chamber, for creating a pressure differential suitable for causing the transport of fluid or other material.

In some embodiments, the vacuum source is a component that a user may actuate to generate a vacuum. Vacuum sources that are actuated to generate a vacuum may be purely mechanical, or may require electricity to operate (e.g. battery-operated or wired to receive electricity from a wall socket). In some embodiments, the vacuum source has a moveable component such as a flexible membrane, a piston, an expandable foam, or a shape memory material that is moved to generate a vacuum. In some illustrative embodiments described in further detail below, the vacuum source may be a flexible dome that is compressible and may be biased to return to an expanded state, generating vacuum during reversion of the dome from a compressed state to an expanded state.

In some embodiments, the vacuum source may be actuated a single time to generate sufficient vacuum. In other embodiments, the vacuum source may be repeatedly actuated (e.g. repeated pumping) to generate the desired vacuum.

In some embodiments, the vacuum source is a pre-packaged vacuum—a volume or chamber that has been pre-evacuated at manufacturing to be at a pressure that is less than ambient pressure. In some embodiments, a user may actuate the fluid receiving device to open fluid communication to the pre-packaged vacuum chamber. In some cases, the pre-packaged vacuum is present in the device before the device is affixed to the skin of the subject to which blood is to be withdrawn. Thus, when the device is first applied to the skin, a pre-packaged vacuum is already present in the device, as opposed to devices in which the device must be first applied to the skin before a vacuum can be created within the device. However, in other embodiments, the device need not have a pre-packaged vacuum.

Thus, the device in some embodiments contains a “pre-packaged” vacuum chamber, such that it is received “ready for use,” without requiring any actuation to produce a vacuum within the vacuum chamber. In some embodiments, the vacuum source is a Vacutainer™ tube, a Vacuette™ tube, or other commercially-available vacuum tube.

In some embodiments, the device operation is entirely mechanical and does not require a power source (e.g. electrical, battery) or software electronics. In other embodiments, however, power sources or software electronics may be used.

In some embodiments, the vacuum source may be a vacuum pump that is able to create a vacuum within the device. In some embodiments, the vacuum source may include chemicals or other reactants that can react to increase or decrease pressure which, with the assistance of mechanical or other means driven by the reaction, can form a pressure differential. In some embodiments, chemical reaction can drive mechanical actuation to form a pressure differential without a change in pressure based on the chemical reaction itself. In some case, the vacuum source may be mechanically generated, for example, using a flexible dome, e.g., as described herein.

Other examples of a vacuum source include: a syringe pump, a piston pump, a syringe, a bulb, a Venturi tube, manual (mouth) suction. In some embodiments, a vacuum source comprises a spring-loaded mechanism. The user may cock the spring-loaded mechanism during use of the device. In other embodiments, the spring-loaded mechanism may be supplied pre-cocked prior to device actuation, and a user may release the mechanism by, e.g., actuating a device actuator. In some embodiments, a vacuum source may comprise a bi-stable dome. The dome may be supplied in a buckled state prior to device actuation, and actuation of the device may cause the buckled dome to pop up into an expanded state to generate vacuum.

In some embodiments, a vacuum may be created by a vacuum source without an external power and/or an external vacuum source, e.g., the vacuum source may be self-contained within the device. For instance, a vacuum may be created through a change in shape of a portion of the device (e.g., using a shape memory polymer). As a specific example, a shape memory polymer may be shaped to be flat at a first temperature (e.g., room temperature) but curved at a second temperature (e.g., body temperature), and when applied to the skin, the shape memory polymer may alter from a flat shape to a curved shape, thereby creating a vacuum. As another example, a mechanical device may be used to create the vacuum, For example, springs, coils, expanding foam (e.g., from a compressed state), a shape memory polymer, shape memory metal, or the like may be stored in a compressed or wound released upon application to a subject, then released (e.g., unwinding, uncompressing, etc.), to mechanically create the vacuum.

Non-limiting examples of shape-memory polymers and metals include Nitinol, compositions of oligo(epsilon-caprolactone)diol and crystallizable oligo(rho-dioxanone)diol, or compositions of oligo(epsilon-caprolactone)dimethacrylate and n-butyl acrylate.

In some embodiments, the device may include an indicator that provides an indication of the vacuum level that has been generated. In some embodiments, the indicator comprises a button or other element that retracts into the vacuum source (e.g. a vacuum chamber) due to being subjected by the vacuum generated by the vacuum source. In some embodiments, the indicator comprises a manometer or other pressure gauge.

According to one aspect, to promote withdrawal of fluid, the device includes an interface configured to contact skin, the interface being conformable to skin, e.g. the interface is able to deform to conform to skin as the skin bulges under vacuum. The interface may conform to the skin in an elastic (e.g. reversible) manner, or in a plastic (e.g. irreversible) manner.

In some embodiments, the interface may be conformable to skin due to the material that the interface is made from, and/or due to structural geometry of the device.

In some embodiments, the interface may be moveable relative to other components of the device, such as a rigid housing or a rigid support that connects the interface to other components of the device.

In some embodiments, the interface is made of a flexible material such as silicone, including ECOFLEX 10, ECOFLEX 30, DRAGONSKIN 30, SMOOTH-SIL 940, SMOOTH-SIL 950, and SMOOTH-SIL 960, each from SMOOTH-ON, INC. Any of these silicone materials may be combined with SLACKER (from SMOOTH-ON, INC.), a material that makes the silicone materials softer and tackier. In some embodiments, the interface is made of a thermoplastic elastomer, including thermoplastic vulcanizate. Examples of thermoplastic elastomers include, but are not limited to, SANTOPRENE 111-35, SANTOPRENE 211-35, SANTOPRENE 111-45, and SANTOPRENE 211-45 from EXXONMOBIL, or VERSAFLEX CL2242, VERSAFLEX CL2250, VERSAFLEX OM 1040X-1, and VERSAFLEX OM 1060X-1 from POLYONE. Examples of other possible flexible materials for the interface include, but are not limited to: polyurethanes, polystyrene/rubber block copolymers, e.g. Styrene-ethylene-butylene-styrene (SEBS), EPDM, and compressible foam (e.g., closed-cell foam or open-cell foam with a thin film coating to provide a seal against the skin).

In some embodiments, the structural geometry of the device may permit the interface to be conformable to skin, (e.g., in some embodiments, moveable relative to other components of the device). For example, the device may include a region of decreased thickness that connects the interface to the rest of the device, and the decreased thickness may act as a flexure region, e.g. a hinge, that permits movement of the interface relative to other portions of the device. Other approaches may be used to achieve a flexure region, such as strategic removal of material (e.g. slits in the interface material), texturing of the material, co-molding of parts (e.g. two rigid materials connected by a more flexible middle material, arranged, for example, in concentric rings or a layered laminate), forming the interface out of components(s) having non-uniform material properties, or a bellows design. In some embodiments, the interface may comprise a single part, or may comprise multiple parts. In some embodiments, the multiple parts may be moveable relative to one another, e.g. via a pivot relationship.

In some embodiments, an interface may be paired with a support that may connect the interface to other components of the device, such as a vacuum source or a device housing.

In some embodiments, the support may be more rigid than the interface. In some embodiments, the interface is made of a first material and the support is made of a second material, the first material having a lower Young's modulus than a Young's modulus of the second material. In other embodiments, however, the support and the interface may be made from the same material.

In some cases, the Young's Modulus of the first and/or the second material may each independent be less than 30 GPa, less than 20 GPa, less than 10 GPa, less than 5 GPa, less than 3 GPa, less than 2 GPa, less than 1 GPa, less than 500 MPa, less than 300 MPa, less than 200 MPa, less than 100 MPa, less than 50 MPa, less than 30 MPa, less than 20 MPa, less than 10 MPa, less than 5 MPa, etc.

In some of these embodiments, structural geometry of the device may permit the interface to be moveable relative to the support. For example, an area of decreased thickness or other shape that gives rise to a hinged arrangement may permit the interface to move relative to the support. In some embodiments, at least a portion of the interface may be thinner than at least a portion of the support.

The shape of the support may vary between different embodiments. In some embodiments, the support is a cylindrical shape with vertically straight walls. In some embodiments, the support is funnel-shaped, where the walls taper in a direction moving from the device opening into the device. In some cases, having a funnel-shaped support may help to distribute forces on the skin and/or may help to promote a desirable skin bulge. The support may have other shapes as well.

In some embodiments, the interface may be rigid rather than flexible. The rigid interface may be provided with one or more features to aid in some of the effects described above, such as promotion of skin recruitment to permit desirable skin bulging, as well as distribution of force on the skin. In some embodiments, a rigid interface may be coated with a lubricant to facilitate movement of skin under the interface. Examples of lubricant include, but are not limited to: petroleum jelly, glycerin, propylene glycol, hydroxyethylcellulose, hydroxypropylmethylcellulose, silicones, e.g. trisiloxane/dimethicone/cyclomethicone, fruit pectin, and extracts of aloe vera.

Materials for the rigid interface include, but are not limited to: photopolymerized methacrylic acid esters, polyethylene terephthalate (alcohol) esters (PET), polypropylene, polyethylene methyl acrylic ester, polycarbonate, polystyrene, poly ethylene, polyvinyl chloride, cycloolefin copolymer (COC), polytetrafluoroethylene, fluoropolymers, polyvinylidene chloride, polyimide, and polyester. Combinations of these and/or other materials may be used in some embodiments.

In some embodiments, the skin may be recruited into the device, as mentioned. In some cases, this may be determined by determining an area where the interface initially contacts the skin, e.g., a first contact region, and determining an area of the skin where the interface contacts it, after withdrawal of fluid, e.g., due to application of reduced pressure. This second contact region may, in some cases, be bigger or circumscribe the first contact region.

In some embodiments, the rigid interface may be shaped to have any of the same shapes as those discussed above for the support portion of the device.

It should be noted that a flow activator need not be included with all embodiments, as the device may not necessarily employ a mechanism for causing fluid release from the subject. For instance, the device may receive fluid that has already been released due to another cause, such as a cut or an abrasion, fluid release due to a separate and independent device, such as a separate lancet, an open fluid access such as during a surgical operation, and so on.

If included, a flow activator may physically penetrate, pierce, and/or or abrade, cut skin either laterally (e.g., slit) or rotationally (e.g., coring), chemically peel, corrode and/or irritate, release and/or produce electromagnetic, acoustic or other waves, other otherwise operate to cause fluid release from a subject. The flow activator may include a moveable mechanism, e.g., to move a needle, or may not require movement to function. For example, the flow activator may include a jet injector or a “hypospray” that delivers fluid under pressure to a subject, a pneumatic system that delivers and/or receives fluid, a hygroscopic agent that adsorbs or absorbs fluid, a reverse iontophoresis system, a transducer that emits ultrasonic waves, or thermal, radiofrequency and/or laser energy, and so on, any of which need not necessarily require movement of a flow activator to cause fluid release from a subject. In some embodiments, the flow activator may include one or more needles and/or blades.

It will be appreciated from the following description that the device may have needle deployment and retraction mechanisms that are conceptually similar in various aspects to the devices disclosed in International Application No. PCT/US2017/043580, filed Jul. 25, 2017, and U.S. Pat. No. 8,821,412, filed Nov. 19, 2012, the disclosures of which are incorporated by reference herein in their entireties.

In addition, the following documents are each incorporated herein by reference in their entities: Int Pat. Apl. Pub. Nos. WO 2010/101621, WO 2011/053787, WO 2011/053796, WO 2011/053788, WO 2011/094573, WO 2011/065972, WO 2011/088214, WO 2010/101626, WO 2011/163347, WO 2012/021792, WO 2012/021801, WO 2012/064802, WO 2011/088211, WO 2012/149143, WO 2012/149155, WO 2012/149126, WO 2012/154362, WO 2012/149134, WO 2016/123282, and WO 2018/022535.

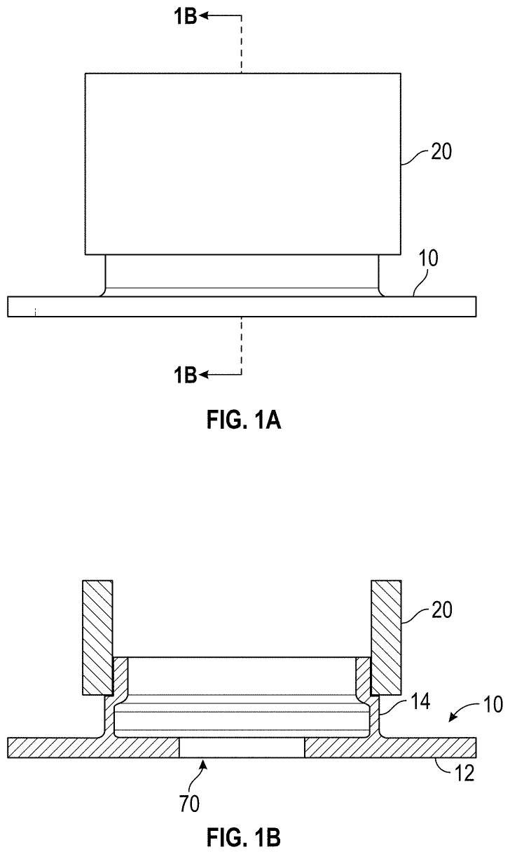

Turning to the figures, FIGS. 1A and 1B depict one illustrative embodiment of a support and interface of a fluid receiving device. The support is cylindrical and, as seen in FIG. 1B, has walls that are straight in the vertical direction. The interface 10 has a horizontal portion 12 and a vertical portion 14. The interface is made of a flexible material such that the horizontal portion 12 is moveable relative to the vertical portion 14 and is also moveable relative to the support 20.

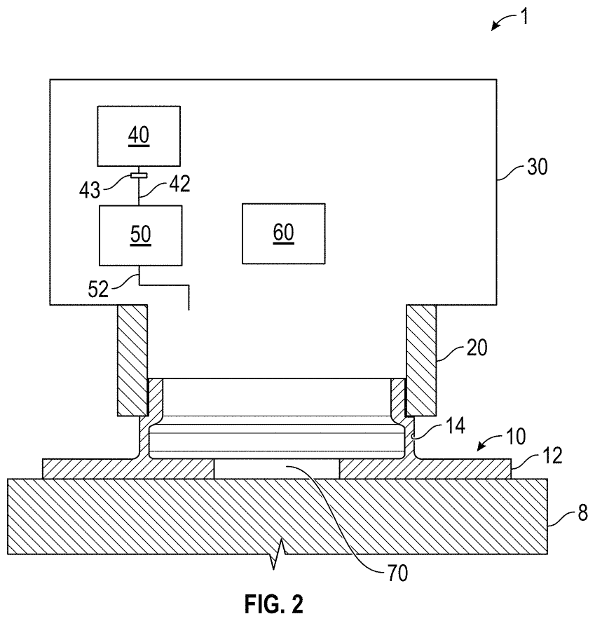

As seen in FIG. 2, the support 20 and interface 10 may be integrated with a fluid receiving module 30 to form a fluid receiving device 1. FIG. 2 depicts the fluid receiving device 1 in contact with a subject's skin 8. The interface 10 defines an opening 70 through which fluid is received from the subject into the device 1. A fluid receiving module can contain various components. In the embodiment shown in FIG. 2, the fluid receiving module 30 includes a vacuum source 40, a storage chamber 50, and a flow activator such as a needle assembly 60. A vacuum pathway 42 may provide fluid communication between the opening 70 and the vacuum source 40, and a storage pathway 52 may provide fluid communication between the opening 70 and the storage chamber 50. In some embodiments, a hydrophobic stop membrane 43 may be positioned between the storage chamber 50 and the vacuum source 40 to prevent fluid withdrawn from the subject from entering the vacuum source 40.

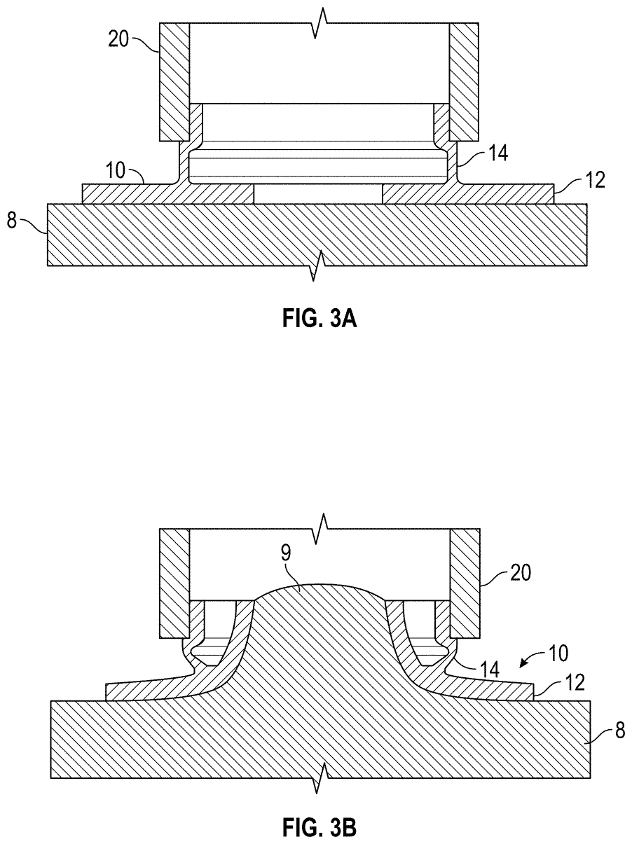

FIGS. 3A and 3B depict interaction between the interface of the fluid receiving device with the skin under atmospheric conditions and under vacuum conditions, respectively. In atmospheric conditions, the horizontal portion 12 of the interface 10 is flush with the skin. Due to the flexibility of the interface 10, if the skin has irregularities, is curved, or otherwise deviates from a flat plane, the interface 10 is able to conform to the shape of the skin to maintain a seal between the interface 10 and the skin.

As shown in FIG. 3B, when the skin is subjected to vacuum, the skin 8 tends to bulge 9 upwardly. Due to the material properties and shape of the interface 10, at least a portion of the interface moves relative to the support 20 to permit the skin to bulge. Part of the horizontal portion 12 flexes upwardly relative to the support 20, permitting the skin to bulge and keeping the interface 10 in contact with the skin 8. The vertical portion 14 of the interface 10 may also flex relative to the support 20 to permit the skin to bulge.

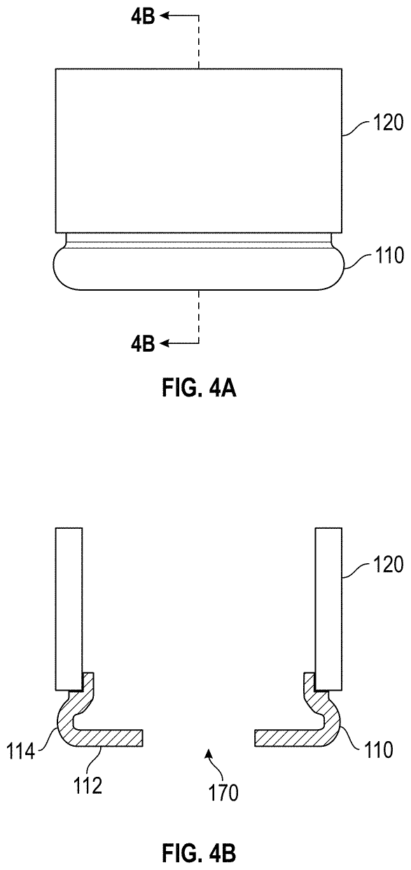

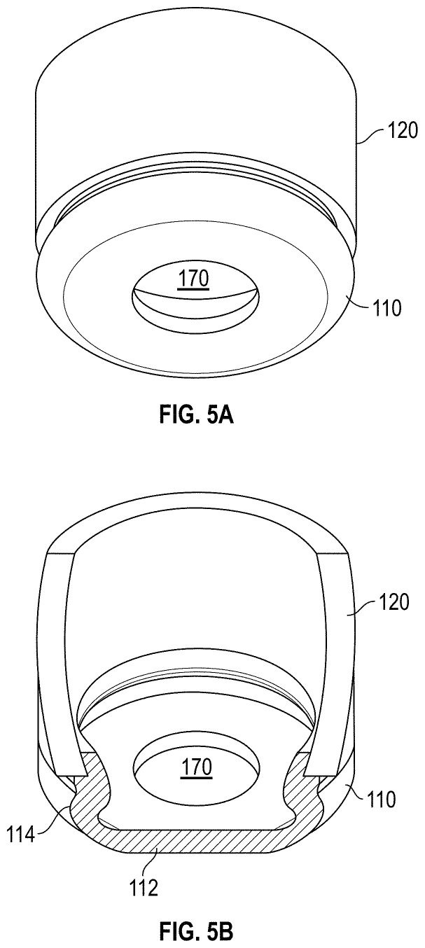

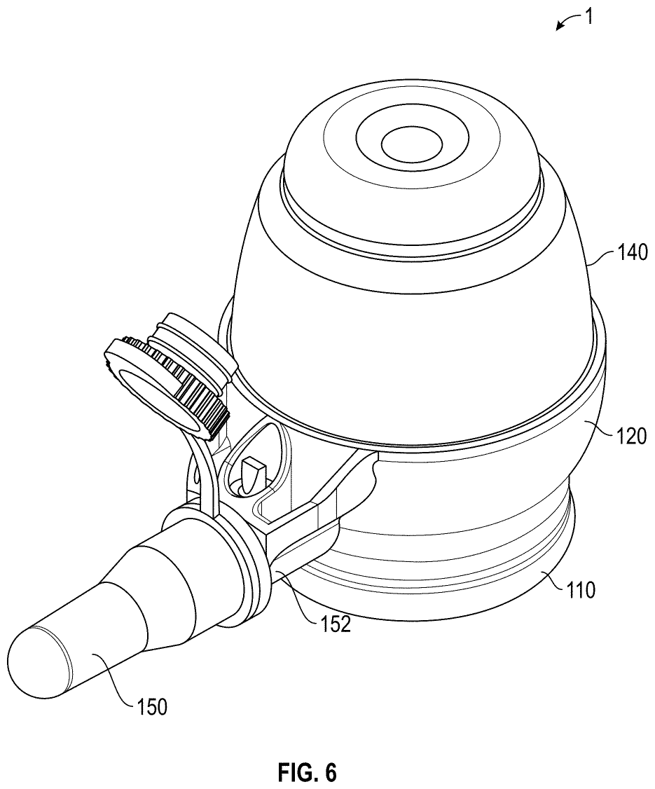

Another illustrative embodiment of a support and interface of a fluid receiving device is shown in FIGS. 4A, 4B, 5A and 5B. The support 120 is cylindrical with walls that are vertically straight. As best seen in FIGS. 4B and 5B, the interface 110 has a horizontal portion 112 and a rounded C-shaped portion 114 that transitions the interface from the support 120 to the horizontal portion 112 of the interface 110. The C-shaped portion 114 may serve as a hinge that permits inward and/or upward flexure of the horizontal portion 112 relative to the support 120 when vacuum is applied. An opening 170 is defined by the interface. In some embodiments, the interface 110 is not axisymmetric. In some embodiments, the horizontal portion 112 may be tapered, e.g. the horizontal portion 112 may be thinner toward the opening 170. In some embodiments, the horizontal portion 112 may include features such as grooves or ridges to help guide blood, e.g. from the opening 170 toward a storage container (also referred to herein as a storage chamber). The interface 110 may be made of a material that has a lower Young's modulus than a material of the support 120. The interface 110 may be made of a flexible material that permits the interface 110 to move relative to the support 120.

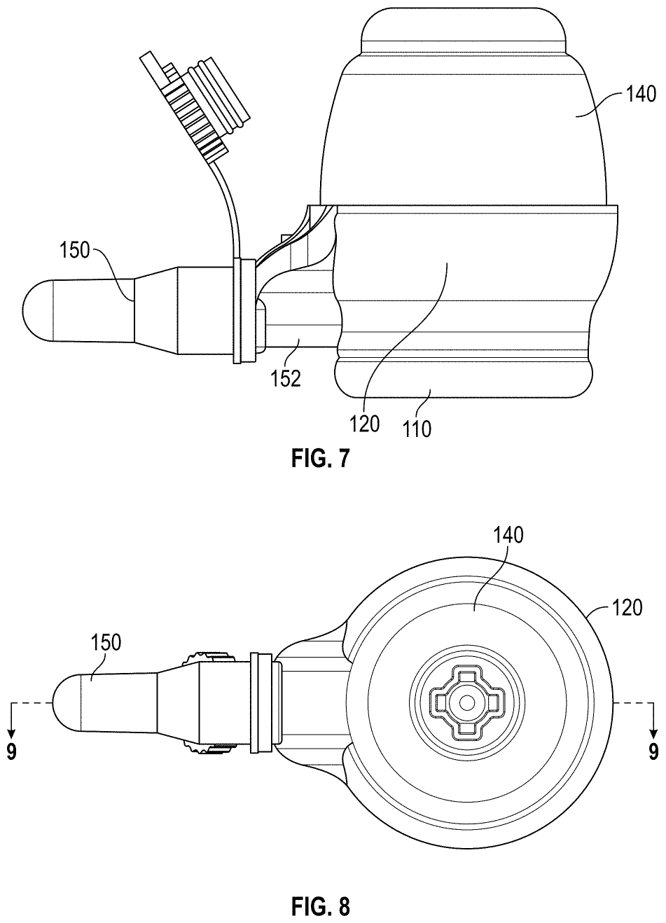

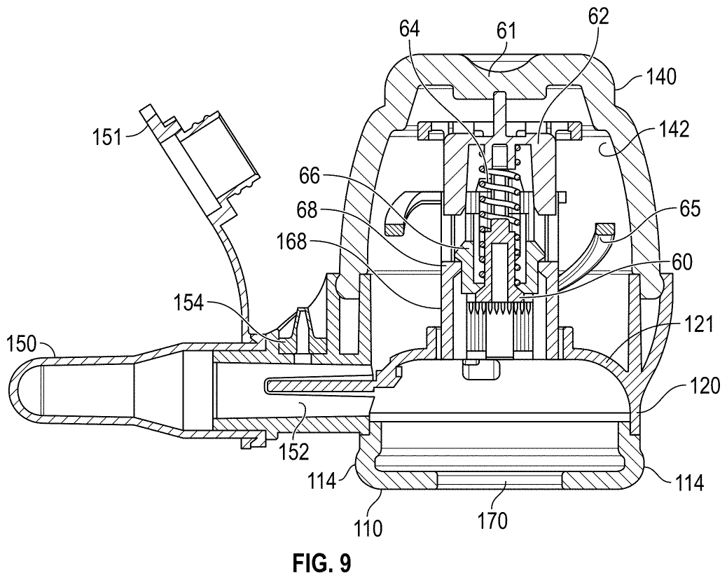

FIGS. 6-9 show the support 120 and interface 110 of FIGS. 4A, 4B, 5A, and 5B integrated with a fluid receiving module to form a fluid receiving device 1. FIG. 9 is a cross-section view of the fluid receiving device of FIG. 6 along line 9-9 of FIG. 8. The fluid receiving module includes a vacuum source in the form of a vacuum bulb 140, a storage container 150, and, as shown in FIG. 9, a piercing assembly (or other flow activator deployment mechanism) including a needle assembly 60 and an actuation mechanism that includes a push cap 62, a latch 66, a spring 64, and a guide housing 168 having a firing ledge 68. The spring 64 may be initially compressed prior to device actuation. The vacuum bulb may be a flexible dome that can change shape when subjected to a force. The vacuum bulb may, in some embodiments, be biased to return to a certain shape when the force applied to the vacuum bulb is removed. In some embodiments, this force may be imparted by the user. In some cases, the vacuum bulb may return to a certain shape under the force imparted by the pressure differential.

In operation, a user depresses device actuator 61, which, in this embodiment, is also a portion of the vacuum bulb 140. Depressing the device actuator 61 causes the push cap 62 to be pushed downwardly, which causes the latch 66 to clear the firing ledge 68, thus freeing the compressed spring 64 to decompress. The spring 64 is coupled to a needle assembly 60 such that decompression of the spring 64 moves the needle assembly 60 in a deployment direction toward the opening 170 and toward the subject's skin, piercing the subject's skin. In some embodiments, when the spring 64 decompresses, it extends to a position past its resting length. Thus, the spring may be at a length that is longer than its resting length during piercing of the subject's skin. After piercing the subject's skin, the spring 64 may self-retract to its resting length, thereby moving the needle assembly 60 upwardly away from the opening 170. Retraction of the needles may serve to prevent subsequent inadvertent piercing of the skin.

In some embodiments, the device may also include a second spring 65. The second spring 65 may be coupled to the needle assembly 60. In some embodiments, the stiffness of the second spring 65 is less than the stiffness of the spring 64 of the actuation mechanism. The second spring 65 may serve as a retraction actuator in the form of a retraction spring. The second spring 65 may be positioned between the housing 168 and a surface 121 and compressed between the housing 168 and the surface 121 during device actuation. In some embodiments, the second spring 65 is attached to the housing 168 and is permitted to slide along surface 121, e.g. during compression.

The second spring 65 may be in contact with a portion of the push cap 62 and the surface 121. In some embodiments, the piercing assembly, which includes the needle assembly 60, latch 66, push cap 62, spring 64 and housing 168, is moveable relative to the support 120. Pushing down on the device actuator 61 causes the push cap 62 to move downward. In this illustrative embodiments, springs 64, 65 are arranged as springs in series. Downward movement of the push cap 62 causes both springs 64, 65 to compress due to the reaction force of surface 121. Spring 65, being less stiff than spring 64, compresses a greater distance than spring 64 in this first stage of actuation.

As the second spring 65 compresses, the entire assembly consisting of the push cap 62, the spring 64, the latch 66, and the housing 168 translates downward toward the opening 170 as a unit until a bottom surface of the housing 168 contacts the subject's skin, or contacts the surface 121, whichever occurs first. As a user continues to push down on the actuator 61 while the bottom surface of the housing 168 is in contact with skin (or in contact with the surface 121), the spring 64 continues to compress. Compression of the spring 64 permits the push cap 62 to move toward the latch 66 and the firing ledge 68 until the push cap 62 contacts the latch 66, squeezing the arms of the latch 66 radially inward. As a result, the latch 66 clears a ledge of the housing 168, thus freeing the compressed spring 64 to decompress. The spring 64 is coupled to a needle assembly 60 such that decompression of the spring 64 moves the needle assembly 60 in a deployment direction toward the opening 170 and toward the subject's skin, piercing the subject's skin. In some embodiments, the spring 64 is coupled to the push cap 62. In some embodiments, when the spring 64 decompresses, it extends to a position past its resting length. Thus, the spring may be at a length that is longer than its resting length during piercing of the subject's skin. After piercing the subject's skin, the spring 64 may self-retract to its resting length, thereby moving the needle assembly 60 upwardly away from the opening 170. Retraction of the needles may serve to prevent subsequent inadvertent piercing of the skin.

In some embodiments, due to the difference in stiffness between the spring 64 and the second spring 65, decompression of the spring 64 during deployment of the needle assembly 60 may cause compression of the second spring 65 against a surface 121. As the spring 64 self-retracts to its resting length, the second spring 65 may also decompress, moving the needle assembly 60 in the retraction direction. In some embodiments, decompression of the second spring 65 occurs only when the user releases the actuator 61. However, in other embodiments, retraction may occur automatically without requiring user release of the actuator.

In some cases, due to the differences in stiffness, spring 65 may compress significantly more than spring 64. The discrepancy in stiffnesses may be such that the housing 168 will reach the skin before spring 64 has compressed to the point that the push cap 62 disengages the latch 66 from the ledge 68 of the housing 168. Disengagement of the latch results in decompression of the spring 64.

As discussed above, device actuator 61 is also a portion of the vacuum bulb 140. When the user applies a force on the device actuator 61 to depress the device actuator 61, this application of force causes the vacuum bulb to flex and move downwardly, decreasing the volume of space 142 located under the vacuum bulb 140. Decreasing the volume of space under the vacuum bulb will initially create a pressure increase inside the device, but pressure will not build up inside the device due to the presence of a vent. Pressure may escape out through the vent. In some embodiments, the vent is a one-way vent. In some embodiments, the vent in the form of a valve 154. The valve 154 may be a one-way valve such that airflow moves only from inside the device to the outside of the device, but not the other way around.

For this and all other embodiments disclosed herein, examples of one-way valves include, but are not limited to, duckbill valves, ball check valves, umbrella valves, dome valves, Belleville valves, diaphragm check valves, swing check valves, stop-check valves, lift-check valves, in-line check valves, cross-slit valves, or any other suitable valve that allows fluid to pass in one direction only.

In some embodiments, the user's action may form part of a valve. For example, a gasket may be operably linked to a device actuator. Prior to device actuation, the gasket may be in a closed position. Actuation of the device actuator may cause the gasket to open and vent air.

It should be appreciated that, a valve may not be necessary in all embodiments. For example, in some embodiments, the vacuum bulb may be pre-assembled within the device in a compressed state prior to device actuation.

In some embodiments, the vacuum bulb 140 is biased toward returning to its original shape after application of force upon the vacuum bulb ceases. Thus, when a user stops depressing the device actuator 61, the vacuum bulb 140 begins moving back to its original shape, thereby increasing the volume of space under the vacuum bulb. This volume increase creates a vacuum that promotes flow of fluid from the subject's pierced skin through the opening 170 into the device.

In the illustrative embodiment of FIGS. 6-9, the device operates by deployment of the needles into the skin prior to application of vacuum to the skin.

The creation of vacuum may cause the interface 110 to flex relative to the support 120, e.g. at the C-shaped portion 114.

Fluid that enters the device flows toward the storage container via a flow passage 152. In some embodiments, the storage container 150 may be removable from the rest of the device. The storage container 150 may have a cap 151 to close off the collected fluid within the storage container 150 after the storage container 150 is removed from the rest of the device. The cap may be attached to the storage container 150, e.g. via a living hinge.

The storage container 150 may be configured to removably couple to the device by fitting over the flow passage 152. The storage container 650 may stay coupled with and form a seal with the flow passage 152 via, for example, an interference fit.

In some embodiments, the storage container is in the form of a collection tube. The storage container may be sized and shaped for compatibility with other devices (which may, e.g. be commercially available from other parties), such as centrifuges, assay devices, or other analysis machines.

In some embodiments, the storage container contains one or more substances or objects prior to actuation of the device, and prior to entry of fluid from the subject into the storage container. For example, in some embodiments, the storage container may contain: sodium heparin, lithium heparin, balanced heparin, dipotassium EDTA, tripotassium EDTA, clot activator (such as silica), sodium citrate, sodium fluoride, sodium oxalate, acid citrate dextrose, a gel for separation during centrifugation, a mechanical barrier for separation during centrifugation, preservative for nucleic acids (DNA, RNA), any combination of the above, and any other suitable object or substance.

In some embodiments, a device may have two or more storage containers. In some embodiments, the device may require a user action to divert incoming substance(s) from the body to either the first storage container or the second storage container. In other embodiments, the device may automatically direct substances toward the first or second storage chamber.

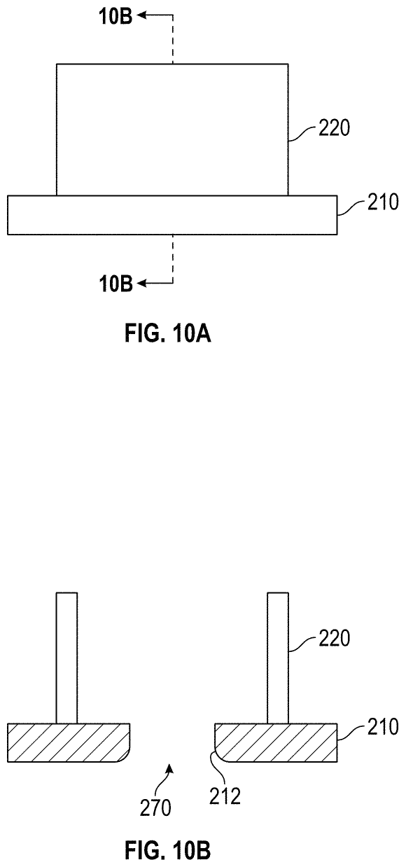

Another illustrative embodiment of a support and interface of a fluid receiving device is shown in FIGS. 10A and 10B. The support 220 is cylindrical with walls that are vertically straight. The interface 210 is horizontal and has a rounded corner 212 at an opening 270. The rounded corner 212 may help to promote movement of skin into the opening 270 while helping to decrease peak pressure applied to the skin.

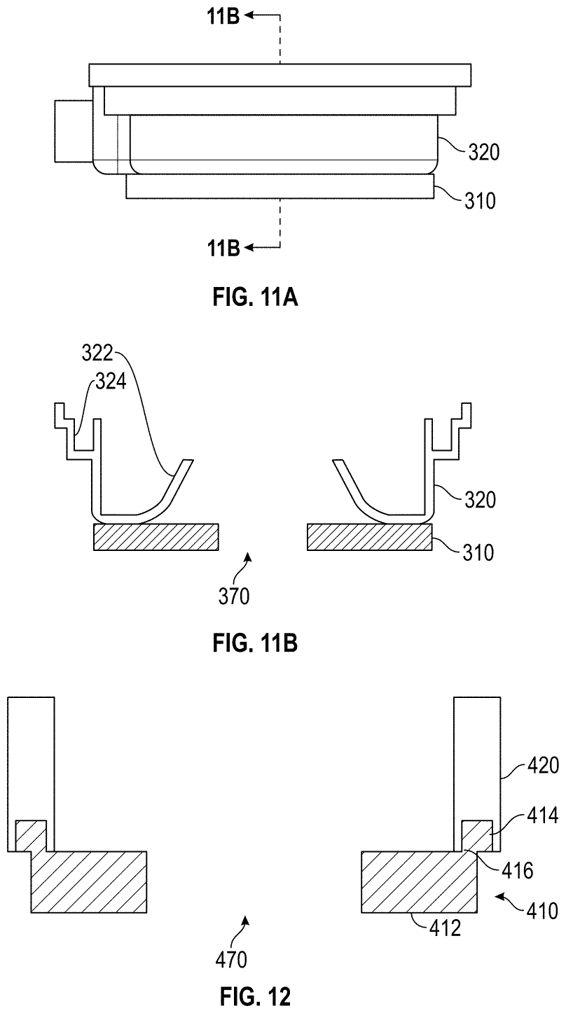

Another illustrative embodiment of a support and interface of a fluid receiving device is shown in FIGS. 11A and 11B. The interface 310 is horizontal and defines an opening 370. The support 320 has a funnel-shaped wall 322 that tapers in a direction moving away from the opening 370 and into the device. The support 320 may, in some embodiments, include an attachment portion 324 to attach the support 320 to the rest of the device, e.g. to a vacuum source or housing.

Another illustrative embodiment of a support and interface of a fluid receiving device is shown in FIG. 12. The support 420 is cylindrical with walls that are vertically straight. The interface 410 is L-shaped with a horizontal portion 412 and a vertical portion 414, where a neck portion 416 joints the horizontal portion 412 to the vertical portion 414. The neck portion 416 may have a width is less than the width of the horizontal portion 412 and the vertical portion 414. The neck portion 416 may facilitate flexing of the horizontal portion 412 relative to the vertical portion 414 and/or relative to the support 420 during vacuum conditions.

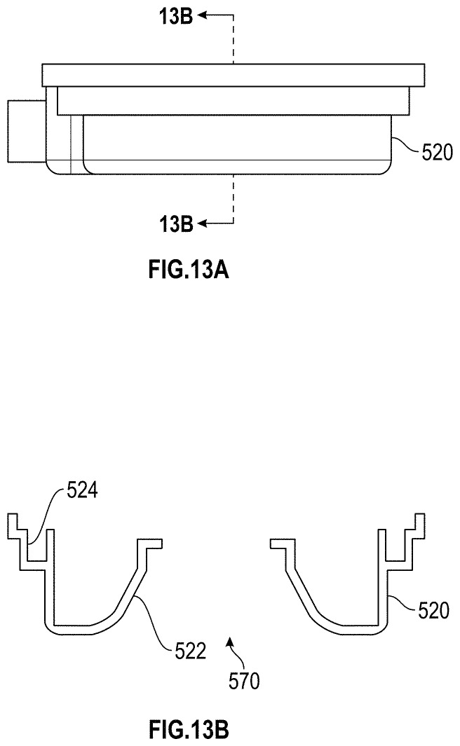

Another illustrative embodiment of an interface of a fluid receiving device is shown in FIGS. 13A and 13B. In this embodiment, the interface is not a flexible component that moves relative to a support or housing. Instead, the interface is a rigid component having a funnel-shaped wall 522 that tapers in a direction moving away from an opening 570 into the device. The interface may include an attachment portion 524 that attaches to a rest of the device, such as a housing or a vacuum source. A lubricant may be applied to the wall 522 to promote skin bulging.

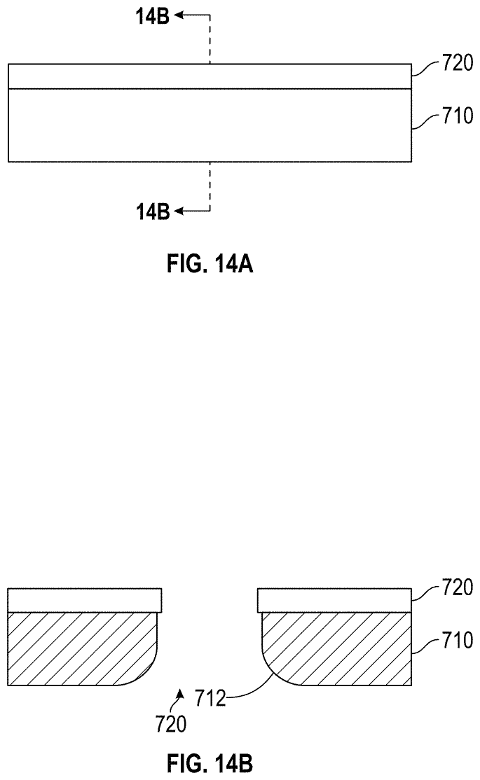

Another illustrative embodiment of a support and interface of a fluid receiving device is shown in FIGS. 14A and 14B. The support 720 is a low-profile cylinder with walls that are vertically straight. The interface 710 is horizontal and has a rounded corner 712 at an opening 770. The rounded corner 712 may help to promote movement of skin into the opening 770. In some cases, this may minimize pressure peaks on the skin.

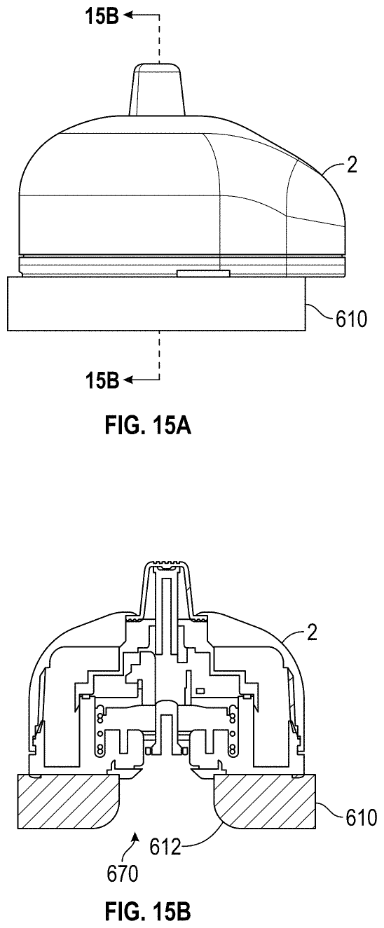

An illustrative example of the flexible interface 710 of FIGS. 14A and 14B being used with a fluid receiving device is shown in FIGS. 15A and 15B. In this embodiment, the flexible interface 610 is used with a device 2 having pre-packaged vacuum, a plurality of microneedles, a snap dome deployment actuator that moves the microneedles to pierce the skin, and a retraction actuator, such as a retraction spring, that moves the microneedles away from the skin. A detailed description of the device 2 may be found in International Application No. PCT/US2017/043580, filed Jul. 25, 2017, and U.S. Pat. No. 8,821,412, filed Nov. 19, 2012.

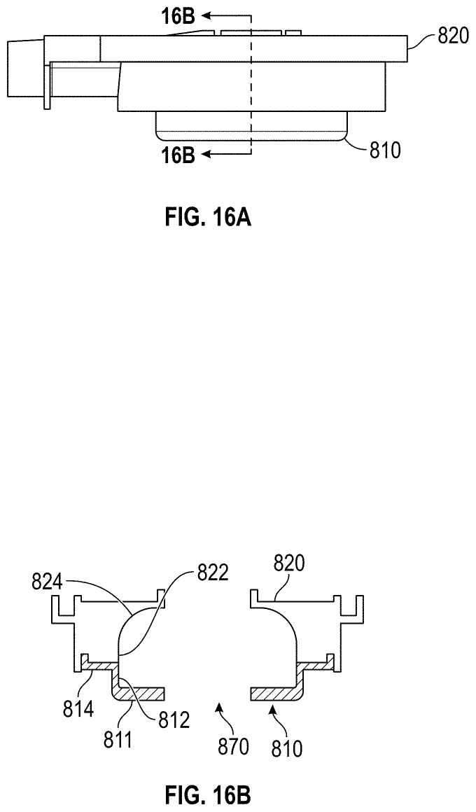

Another illustrative embodiment of a support and interface of a fluid receiving device is shown in FIGS. 16A and 16B. The interface 810 is flexible and includes a first horizontal portion 811, a vertical portion 812 and a second horizontal portion 814. The support 820 has a wall that transitions from a vertical wall 822 to a curved dome 824.

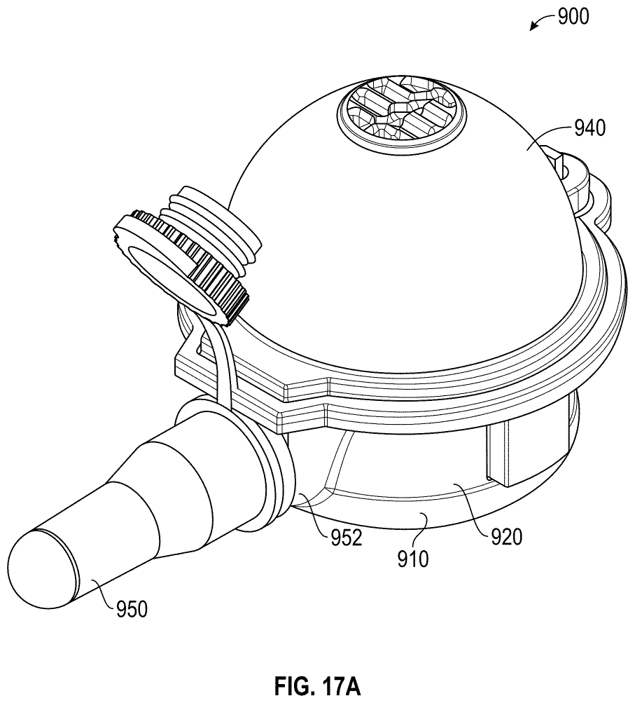

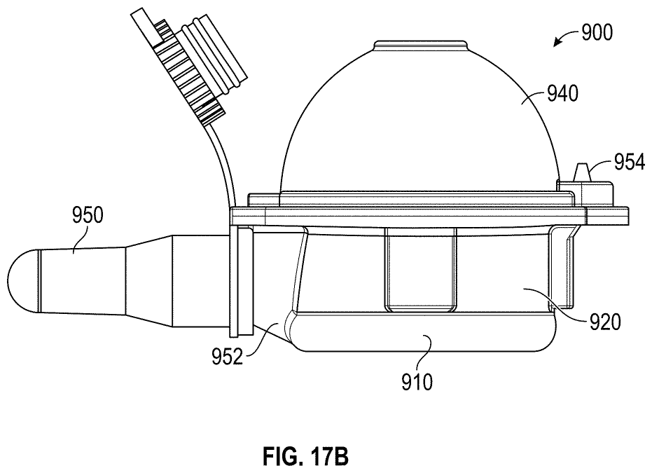

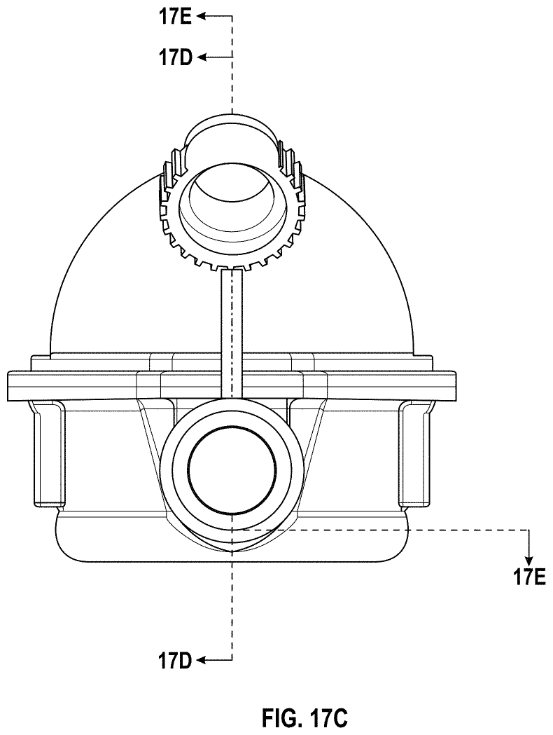

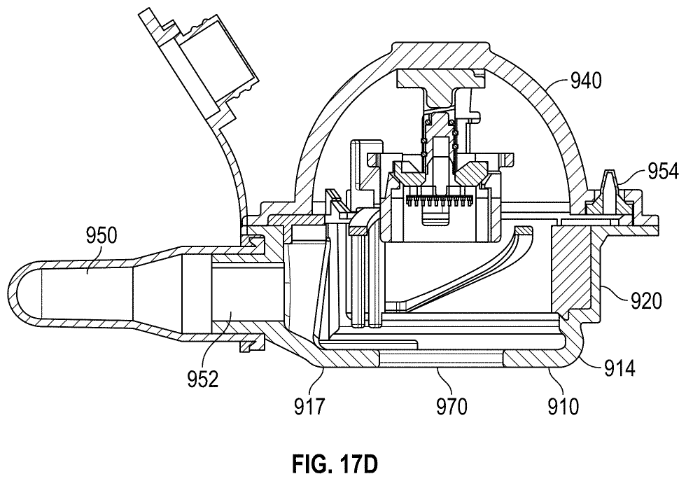

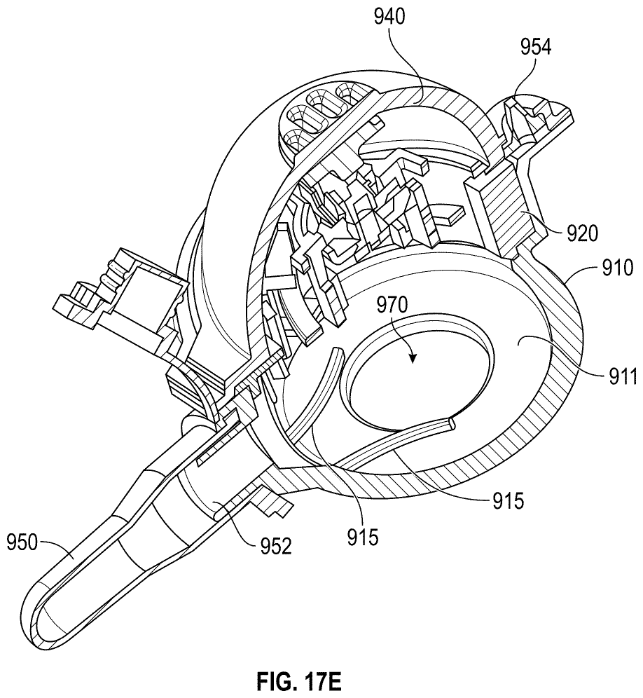

Another illustrative embodiment of a fluid receiving device is shown in FIGS. 17A-17E.

FIG. 17D is a cross-section view of the fluid receiving device of FIG. 17A taken along line 17D-17D in FIG. 17C. FIG. 17E is a partial cutaway view of the fluid receiving device of FIG. 17A taken along line 17E-17E in FIG. 17C.

The device includes an interface 910 and a support 920. In this illustrative embodiment, the interface 910 and the support 920 are made of the same material. As seen in FIG. 17D, support 920 may have a larger thickness than the interface 910. The interface 910 may include a C-shaped portion 914 that permits the interface 910 to be moveable relative to the support 920. The device may have a similar needle deployment, retraction, and vacuum creation arrangement as that described for the embodiment of FIGS. 6-9. In some embodiments, placement of the valve 954 in device 900 may be at a different position than that of the FIGS. 6-9 embodiment. In some embodiments, the valve 954 is located at an end of the device opposite to the storage container 950. In other embodiments, however, the valve 954 may be located at the region of the flow passage 952 into the storage container 950. In some embodiments, the valve may also be a separate part as shown, or it may be incorporated into a single part such as a particular geometry added to the vacuum bulb, or it may be formed by the joining of two components such as the addition of specific geometry and a specific bond pattern between the vacuum bulb and the support.

In some embodiments, the device may include features that help to guide flow of blood toward the storage chamber. As seen in FIG. 17E, the device includes channels 915 extending from the device opening 970 toward the flow passage 952 into the storage container 950. The channels 915 may be formed into an internal surface 911 of the interface 910.

In some embodiments, a portion of the interface 910 that transitions to the flow passage 952 into the storage container 950 may be shaped as a ramp 917 to aid in flow of blood toward the storage container 950. As a result, the interface 910 may be non-symmetric. As seen in FIG. 17D, one side of the interface 910 may have a C-shaped portion 914 and the other side of the interface may be shaped as a ramp 917.

As discussed above, in some embodiments, the device actuator may be a portion of the vacuum source, such as the vacuum bulb described above.

According to one aspect, in some embodiments, a fluid receiving device may have a component that contacts and compresses the vacuum source rather than having the user contact the vacuum source directly. It is appreciated that such a component may, in some embodiments, aid in compression of the vacuum source, e.g., may aid in complete, uniform, and/or consistent compression of the vacuum source.

In some embodiments, the component that contacts and compresses the vacuum source is attached to a device actuator that is distinct from the vacuum source. In some embodiments, the device actuator has a stem and a user-contacting portion, also referred to herein as a button. In some embodiments, the user-contacting portion and the stem are fixed to one another such that movement of one part moves the other, and vice versa. As a user presses down on the user-contacting portion, the stem may also contact and compress the vacuum source.

In some embodiments, the fluid receiving device includes a shell that constrains movement of the device actuator to aid in compression of the vacuum source by the device actuator. In some embodiments, the shell constrains movement of the device actuator to linear movement. The shell may be made of a material having a higher Young's modulus than the material of the vacuum source.

In some embodiments, the shell has an opening through which the device actuator moves. The stem of the device actuator may move through the opening of the shell. The opening and stem may be complementarily sized and shaped to constrain movement of the device actuator to linear movement.

In some embodiments, the fluid receiving device includes a ratchet mechanism that permits movement of the device actuator relative to the shell in one direction while resisting movement of the device actuator in the opposite direction. The ratchet mechanism may promote complete compression of the vacuum source by prohibiting the vacuum source from returning to its original shape until the vacuum source has been completely compressed. In some embodiments, the ratchet mechanism includes a ratchet located on the device actuator, and a pawl located on the shell, or vice versa. In some embodiments, the fluid receiving device includes a lockout that prohibits subsequent actuation of the device if the device has been previously actuated. In some embodiments, such a lockout may be provided by the ratchet mechanism. In some embodiments, the ratchet mechanism may be located on components other than, or in addition to, the device actuator.

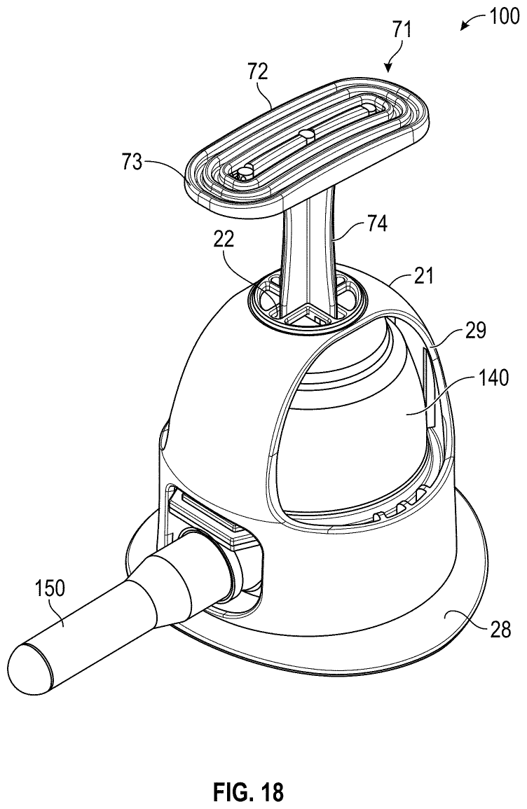

An illustrative embodiment of a fluid receiving device 100 with a device actuator 71 that is distinct from a vacuum source 140 is shown in FIG. 18. In this illustrative embodiment, the vacuum source is a flexible dome that has a first shape prior to compression and a second shape during compression. The flexible dome is biased to return to its first shape when the flexible dome is no longer subjected to compression.

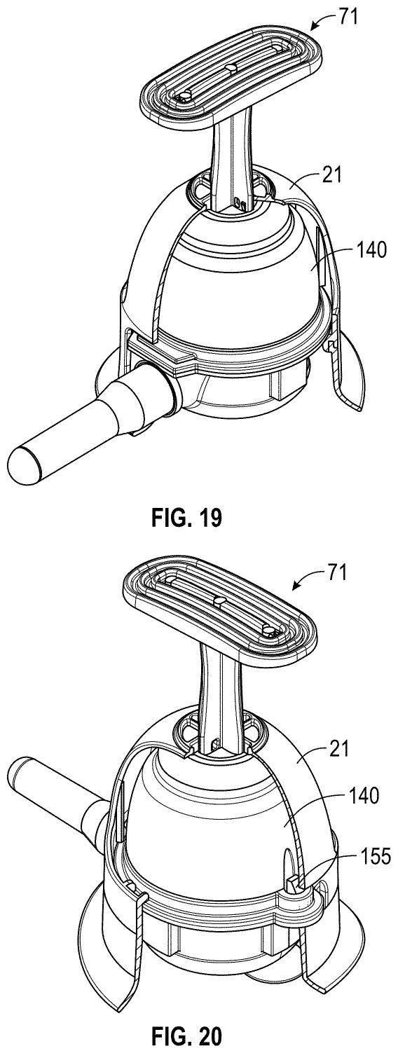

The device actuator 71 includes a user-contacting portion 72 and a stem 74. The fluid receiving device also includes a shell 21 having an opening 22 that receives the stem 74. The shell 21 may be made of a material having a higher Young's modulus than the material of the flexible dome 140. The stem 74 is moveable relative to the shell 21 through the opening 22. The shell 21 may, in some embodiments, include a bottom rim 28. The rim may help to stabilize the device against the skin during use. In some embodiments, the shell 21 may include one or more window openings 29 through which the vacuum source 140 may be visualized by a user. A front perspective partial cutaway view of the fluid receiving device is shown in FIG. 19, and a rear perspective partial cutaway view of the fluid receiving device is shown in FIG. 20. In these partial cutaway views, a portion of the shell 21 is hidden from view to reveal more of the vacuum source 140 beneath.

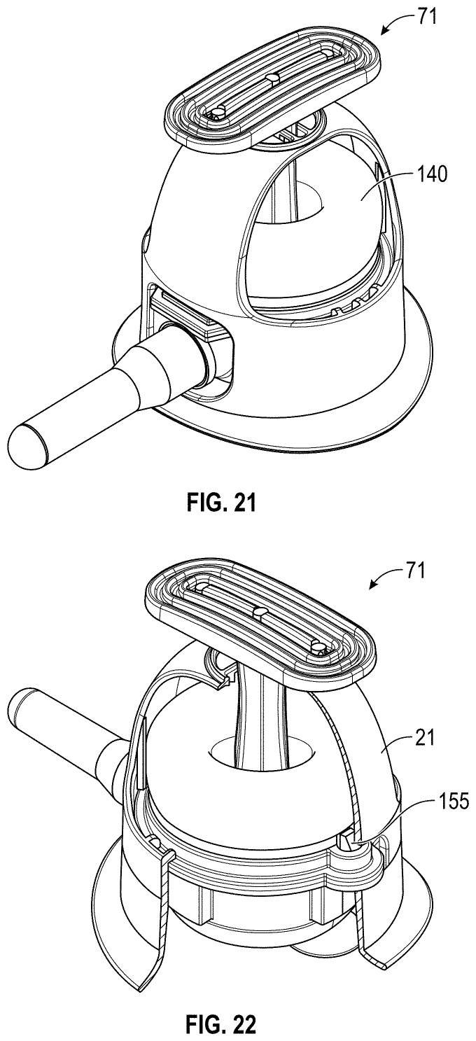

When the user applies a force on the device actuator 71, this application of force causes the bottom of the stem 74 to press against the flexible dome 140, thus causing the flexible dome 140 to compress by flexing downwardly, as shown in FIGS. 21 and 22.

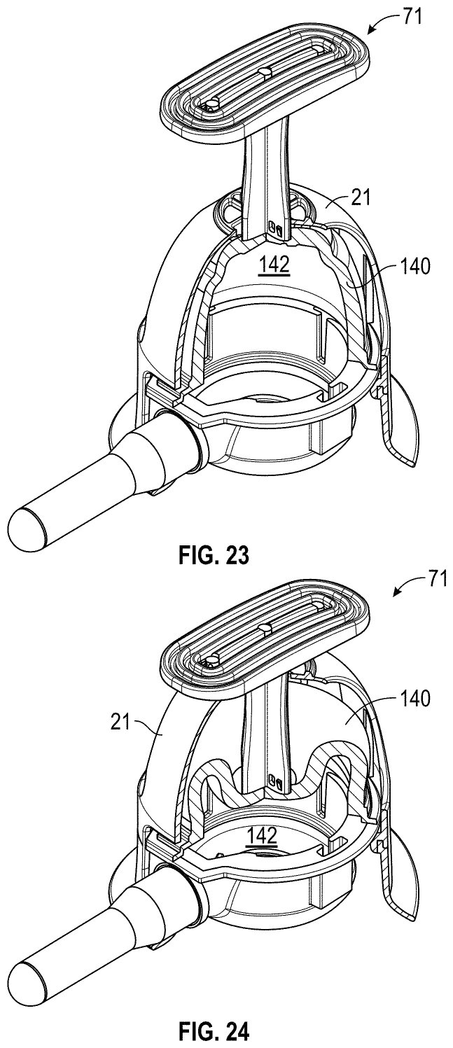

FIG. 23 is a partial cutaway view of the fluid receiving device prior to actuation of the device actuator, where a portion of the shell and a portion of the flexible dome are hidden from view. The piercing assembly is also hidden from view for a clearer view of the inside of the flexible dome 140. In FIG. 23, no force is being applied to the device actuator 71, and the flexible dome 140 is in its first, original shape. As seen in FIG. 23, a volume of space 142 is located under the flexible dome 140. In FIG. 24, the device actuator 71 has been pressed down by a user, causing compression of the flexible dome 140, which decreases the volume of space 142 under the flexible dome 140. Decreasing the volume of space 142 under the flexible dome 140 will initially create a pressure increase inside the device, but pressure will not build up inside the device due to the presence of a valve 155, shown in FIGS. 20 and 22. Pressure escapes out through the valve 155. The valve may 155 be a one-way valve that permits air under the flexible dome 140 to exit as the vacuum source 140 is being compressed, and prohibits air from moving back in the opposite direction. In FIG. 24, the device actuator 41 has been pushed all the way to its bottomed out position, and the flexible dome 140 is in its second shape.

When a user ceases applying force to the device actuator 71, the flexible dome 140 may be free to return to its original shape. Return of the flexible dome from the second shape back to the first shape increases the volume of space under the flexible dome 140, thereby creating vacuum under the flexible dome 140.

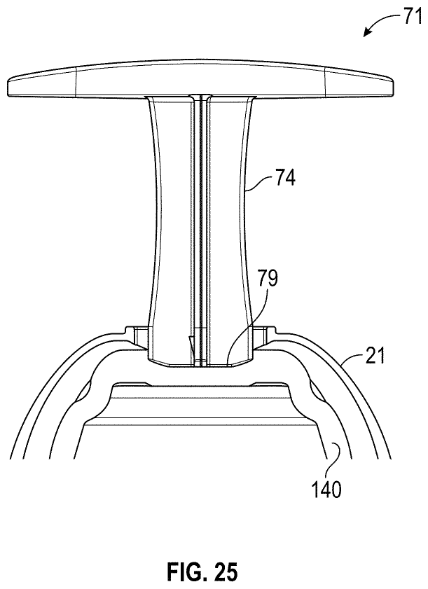

The interaction between the device actuator, shell and flexible dome is shown in the cross-section view of FIG. 25. In this illustrative embodiment, the stem 74 of the device actuator 71 has a contact surface 79 that is in direct contact with the flexible dome 140. However, in other embodiments, one or more intermediate components may be located between the stem and the flexible dome, and such intermediate components may transmit force applied to the device actuator to the flexible dome.

In some embodiments, the stem 74 and the flexible dome 140 are in contact but are not attached to one another. In other embodiments, however, the stem 74 and flexible dome 140 are attached to one another, e.g. via interference fit, heat staking, adhesive, mechanical interlock, or any other suitable attachment arrangement. The device actuator 71 slides within an actuator opening in the shell 21, and the shell 21 overlies the flexible dome 140.

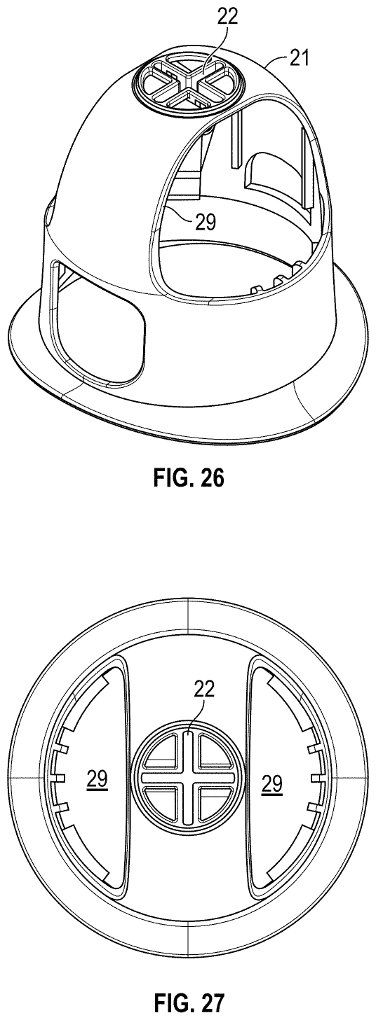

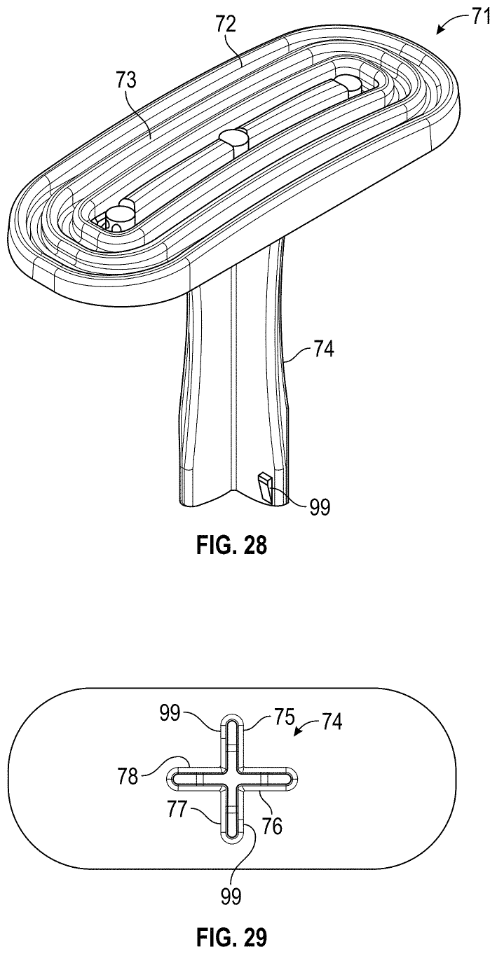

According to one aspect, the shell and actuator opening serves to constrain movement of the device actuator to linear movement, which may aid in centered and uniform compression of the vacuum source. The actuator opening 22 of the shell 21 is shown in the perspective view of FIG. 26 and in the top view of FIG. 27. In this illustrative embodiment, the actuator opening 22 is a plus-sign shape. The shape of the actuator opening 22 corresponds with the shape of the stem 74 of the device actuator 71. A perspective view of the device actuator 71 is shown in FIG. 28, and a bottom view of the device actuator 71 is shown in FIG. 29. As seen in FIG. 29, the stem 74 also has a plus-sign shape, made up of a first fin 75, second fin 76, third fin 77, and a fourth fin 78. The stem 74 is sized and shape to slide linearly through the actuator opening 22 of the shell 21, but is prohibited from rotating or tilting within the actuator opening 22. As such, the device actuator 71 may be constrained to linear movement.

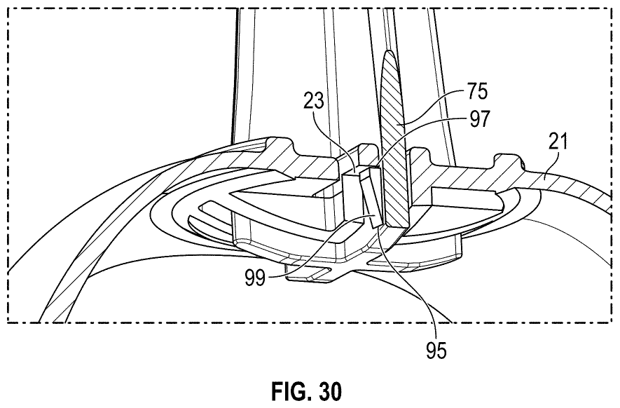

In some embodiments, the stem 74 includes an assembly lockout 99 that permits the stem 74 to be inserted into the actuator opening 22 during assembly, but is shaped to prohibit removal of the stem 74 from the actuator opening 22 afterwards. A close-up view of the interaction between the assembly lockout 99 and the shell 21 is shown in FIG. 30. The assembly lockout 99 is wedge-shaped, with the leading edge 95 (i.e. the point of the wedge) facing the bottom of the stem. If the device actuator 71 is pulled up away from the shell 21, the trailing edge 97 of the assembly lockout 99 abuts against an interior surface 23 of the shell 21, thus prohibiting removal of the device actuator 71 from the shell 21. The stem 74 may have one or more assembly lockouts located on its fins. For example, the stem may have assembly lockouts on all four fins, on only two fins, e.g. two opposing fins or two adjacent fins, and/or assembly lockouts on opposing sides of a fin.

In the embodiment shown in FIG. 28, the device actuator 71 has a user-contacting portion 72 that is configured to be pressed down upon by the user to actuate the fluid receiving device. In this illustrative embodiment, the user-contacting portion 72 includes a plurality of ribs 73 that may provide traction to help a user to press down on the device actuator. The ribs in this illustrative embodiment are a plurality of concentric oval-shaped ribs. However, other arrangements of ribs are possible, such as linear ribs.

In some embodiments, the surface area of the user-contacting portion is larger than the cross-section of the stem. The size of the surface area of the user-contacting portion may help to distribute the applied force from the user. In some embodiments, the ribs may add strength to the user-contacting portion to help distribute and withstand the applied force. In some embodiments, the vacuum source requires the application of a threshold amount of actuation force to generate vacuum. The size of the surface area of the user-contacting portion may allow a user to use more fingers or an entire palm to leverage more muscles in the arm and/or shoulder to overcome the actuation force required to generate vacuum.

According to one aspect, the fluid receiving device includes a ratchet mechanism that resists movement of the device actuator relative to the shell in a certain direction.

In one illustrative embodiment, the ratchet mechanism has a ratchet and pawl, where the ratchet is attached to the stem of the device actuator, and the pawl is attached to the shell. The pawl may be a cantilevered beam that extends from the shell. In some embodiments, the pawl may be integrally formed with the shell as a single monolithic component, i.e. the shell and pawl are formed as one piece at the same time. As one illustrative example, the pawl may be formed by a living hinge with the shell. In other embodiments, the pawl may be formed separately from the shell and then assembled to the shell, e.g. via a mechanical hinge.

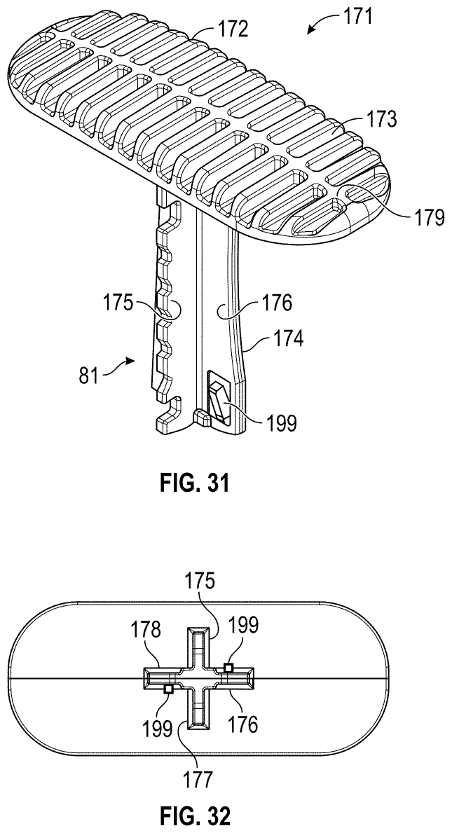

FIG. 31 shows a perspective view of a device actuator 171 having a user-contacting portion 172 and a stem 174. The stem 174 includes a ratchet 81 having a plurality of teeth. As shown in the bottom view of FIG. 32, the stem 174 includes four fins that form a plus-sign shape: first fin 175, second fin 176, third fin 177, and fourth fin 178.

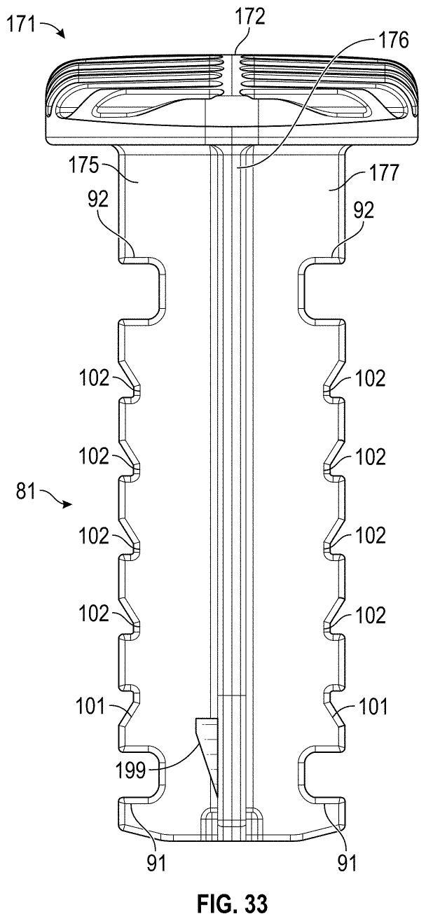

As shown in the side view of the device actuator 171 in FIG. 33, the ratchet 81 is formed on two opposing fins, the first fin 175 and the third fin 177. On each side, the ratchet includes a first tooth 101, and a subsequent plurality of teeth 102. The ratchet also includes first slots 91 toward the bottom of the stem (the end of the stem away from the user-contacting portion 172) and second slots 92 toward a top of the stem (the end of the stem toward the user-contacting portion 172).

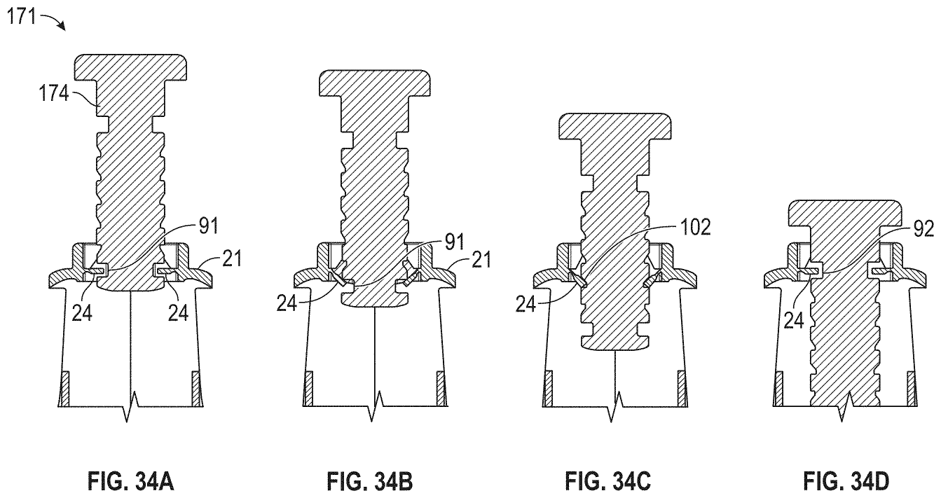

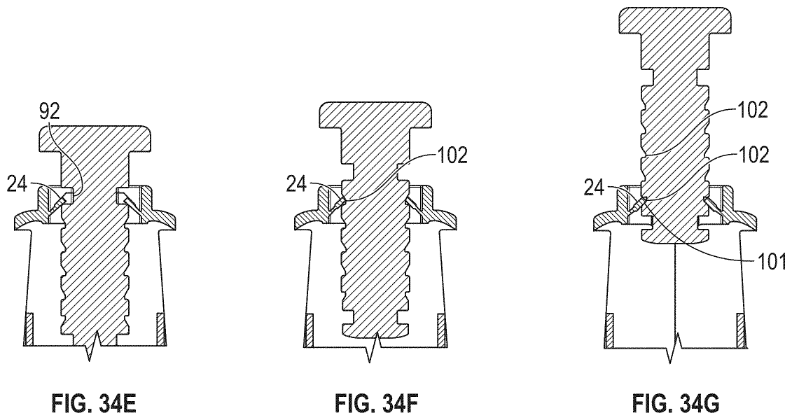

FIGS. 34A-34G depict a sequence of interactions of the ratchet and pawl mechanism as the device actuator is moved relative to the shell. As seen in FIG. 34A, the shell 21 includes two opposing pawls 24 that interact with the ratchet on the device actuator. In FIG. 34A, the device actuator 171 is in its initial position prior to device actuation. In the pre-actuation state, the pawls 24 are located within the first slots 91 at the bottom of the stem. In FIG. 34B, the user continues to push down on the device actuator 171, and the device actuator 171 moves downward relative to the shell 21, causing the pawls 24 to flex downwards as they contact the corners of the first slots 91. In FIG. 34C, the user continues to push down on the device actuator 171 and the pawls engage in the teeth 102, which are shaped to prevent the device actuator 171 from moving in the return direction upward. Because the flexible dome 140 is biased to return to its original, non-compressed shape, when the user stops pushing on the device actuator, without the ratchet, the flexible dome 140 would revert back to its original shape, pressing up on and moving the device actuator upward. If a user were to prematurely release the device actuator prior to complete actuation (e.g., letting go of the device actuator prior to pressing the device actuator all the way down until the device actuator bottoms out), the flexible dome may not have been fully compressed, and thus may not generate as much vacuum as it would have compared to a full compression of the flexible dome. Thus, in preventing the device actuator 171 from moving in the return direction upward, the teeth of the ratchet may help to prevent insufficient generation of vacuum. In addition, because the device actuator remains in a fixed position due to the ratchet holding the device actuator in place, the ratchet may provide a visual indicator to a user that actuation is incomplete.

Next, in FIG. 34D, the user has pushed the device actuator 171 all the way down into its bottomed out position, thus completing actuation of the device. With the device actuator 171 in the bottomed out position, the pawls 24 enter the second slots 92, which free the pawls 24 from the teeth 102, allowing the device actuator 171 to move back upward in the return direction. In FIG. 34E, the user has let go of the device actuator 171, and the device actuator 171 is being pushed upward by the vacuum source reverting back to its original shape. With the corner of the second slots 92 pushing on the pawls 24, the pawls reverse flex direction, flexing upward instead of downward. In FIG. 34F, the device actuator 171 continues to be pushed upward by the vacuum source, as pawls 24 slide freely past the teeth 102 due to the reversed pawl flex direction and angle of the teeth 102. Finally, in FIG. 34G, the pawls 24 reach and engage with the first teeth 101, which have a reversed orientation relative to the other teeth 102. The first teeth 101 are oriented in a manner that prohibits the device actuator 171 from moving downward relative to the shell 21, thus prohibiting subsequent actuations of the device. The ratchet thus includes an actuation lockout feature that prohibits more than one actuation of the device.

It should be appreciated that, in other embodiments, no actuation lockout feature is included and the device may be re-used more than once.

In some embodiments, the ratchet and pawl arrangement serves as a detent that provides tactile and/or auditory feedback to a user during actuation of the device. The user may feel a vibrating sensation and/or hear clicking as the pawls slide against the teeth.

It should be appreciated that, in some embodiments, a ratchet mechanism may be provided on components other than, or in addition to, the device actuator. For example, in some embodiments, a ratchet mechanism may be provided between the support 200 and the guide housing 280. Ratchet teeth may be provided on the support and a pawl may be provided on the guide housing, or vice versa. With a ratchet mechanism between the support and the guide housing, the surface 291 may, in some embodiments, be attached to the flexible dome 140 to couple the ratchet mechanism with the flexible dome. The surface 291 may be attached to the flexible dome 140 e.g. by adhesive, UV welding, mechanical interlock, or any other suitable attachment method.

It should be appreciated that a ratchet is not required in all embodiments. In some embodiments, the device does not include a ratchet mechanism. For example, the device of FIG. 18 may include a ratchet in some embodiments, and may include no ratchet in other embodiments.

In the illustrative embodiment of the device actuator 171 in FIG. 31, the device actuator has a user-contacting portion 172 with a different rib arrangement than that of the FIG. 28 embodiment. In the FIG. 31 embodiment, the user-contacting portion 172 has a plurality of linear ribs 173 on either side of a central raised portion 179.

The device actuator 171 may include an assembly lockout 199 on one or more fins of the stem 174. In some embodiments, the assembly lockout may be attached to the stem via a hinge that permits rotating movement of the assembly lockout 199 when subjected to force in a first direction, but does not permit rotating movement of the lockout when subjected to force in a second direction opposite to the first. In some embodiments, the hinge may be a living hinge.

According to one aspect, the device may include a piercing assembly that is configured to trigger release of needle(s) into skin in response to contact with skin. In some embodiments, the piercing assembly may be arranged in a floating arrangement in which a deployment actuator (e.g. a deployment spring) and needle assembly may move together as one unit in a deployment direction toward the device opening during device actuation. The deployment actuator may be triggered to deploy the needle assembly when a component of the piercing assembly has come into contact with skin. A skin contact-actuated arrangement may help to facilitate successful piercing of the user's skin and prevent premature activation of the device. In some embodiments, skin contact actuation may help to promote consistency of needle insertion across users having different skin characteristics (e.g. users having more compliant versus less compliant skin).

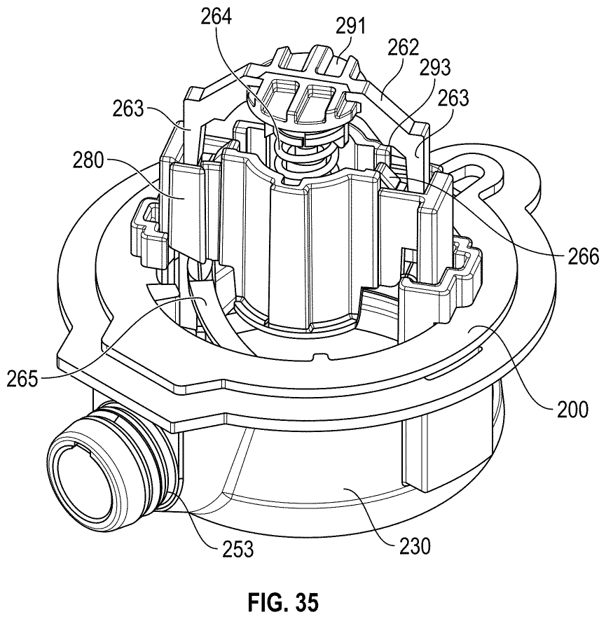

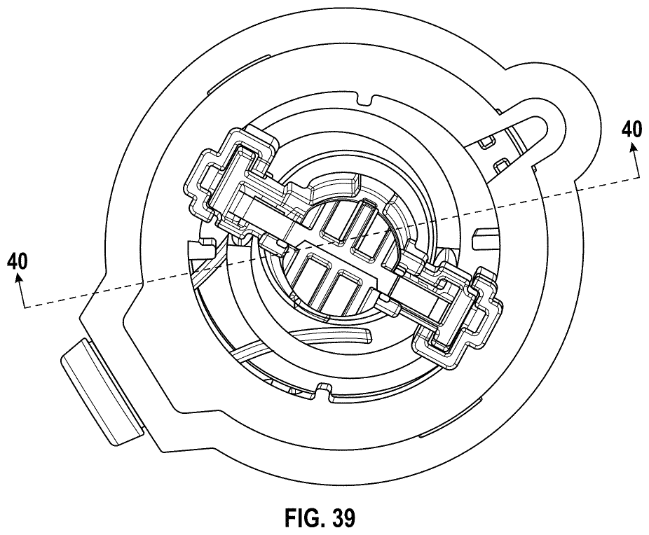

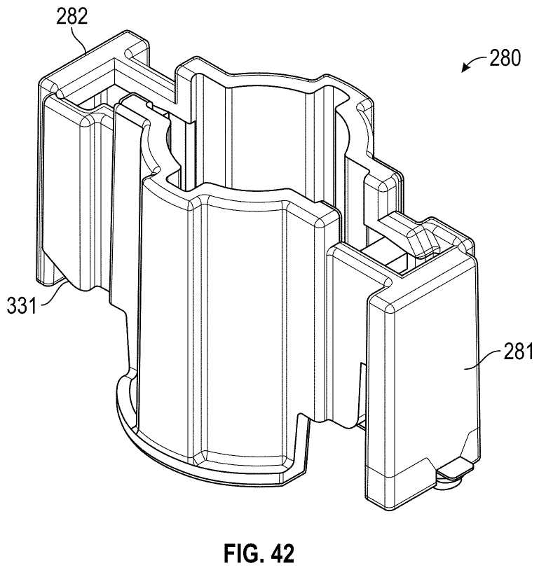

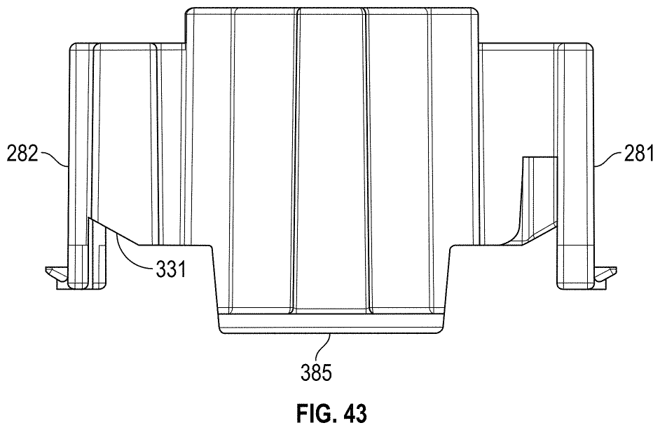

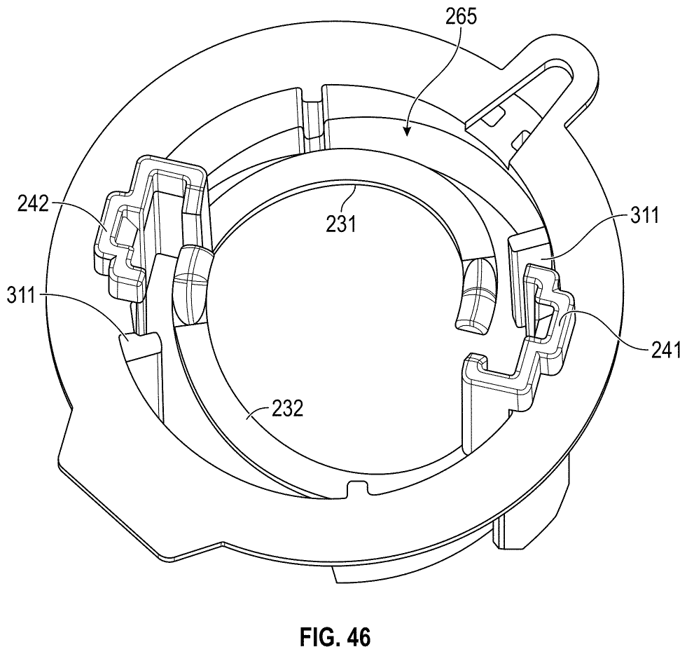

One illustrative embodiment of a piercing assembly of a device is shown in FIGS. 35-47. This piercing assembly may be used with any of the interface configurations described above. In some embodiments, this piercing assembly is positioned within a vacuum source, such as the flexible dome shown and described above. In some embodiments, this piercing assembly is substituted into the devices of the embodiment of FIG. 9, or of FIG. 17D. In some embodiments, this piercing assembly is located within the flexible dome 140 of the embodiment of FIG. 23.

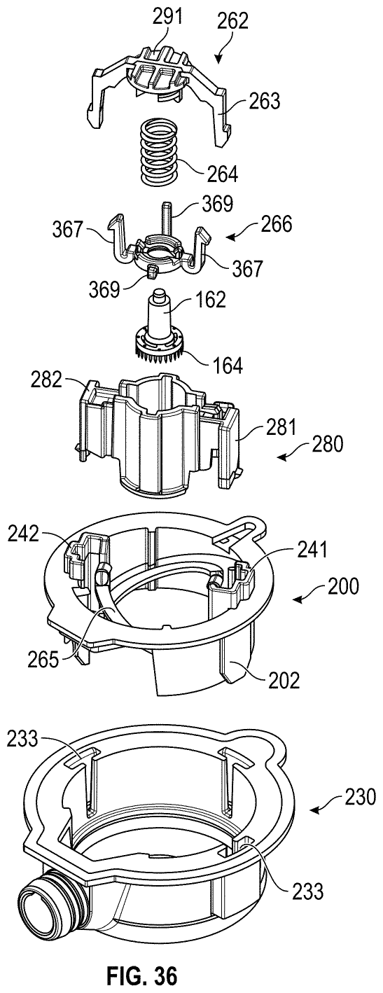

As shown in FIG. 36, the piercing assembly may include a push cap 262 having arms 263. The push cap may include a push surface 291. In some embodiments, during actuation of the device, a bottom inner surface of the flexible dome may come into contact with the push surface 291 and exert a force onto the push surface 291 to initiate needle deployment.

In some embodiments, the piercing assembly may have a deployment actuator in the form of a deployment spring 264, and a retraction actuator in the form of a retraction spring 265. When in a decompressed state, the deployment spring 264 may be positioned between the push cap 262 and the latch 266. When in a decompressed state, the retraction spring 265 may be positioned between the guide housing 280 and a bottom portion of the support 200. In the illustrative embodiment of FIG. 36, the deployment spring 264 is a coil spring. However, other potential energy storing devices may be used. In the illustrative embodiment of FIG. 36, the retraction spring 265 comprises a pair of helical cantilevered arms that extend from a support 200. However, other potential energy storing devices may be used.

In some embodiments, the piercing assembly may have a guide housing 280 that receives the push cap 262, the latch 266, the deployment spring 264, the post 162, and the needles 164. The needles 164 may be configured to be moveable through the guide housing 280 during decompression of the deployment spring 264. The push cap 262, latch 266, deployment spring 264 and post 162 may also be moveable through the guide housing.

In some embodiments, the piercing assembly may have a support 200 and an interface 230. In some embodiments, the interface is made of a first material and the support ring is made of a second material, the first material having a lower Young's modulus than a Young's modulus of the second material. In some embodiments, the support 200 may provide structural stiffness to the less stiff interface 230. In some embodiments, the interface 230 may be made of any of the materials and/or have any of the properties discussed above with regard to interfaces. In some embodiments, the support 200 includes one or more tabs 202 extending from a sidewall of the support. The interface 230 may include corresponding slots 233 that are sized and positioned to receive the tabs 202 of the support 200 to allow the support 200 and interface 230 to interlock to one another. The tabs 202 may aid in decreasing buckling of the sidewall of the interface 230 when the interface is pressed against the subject's skin and/or during vacuum release.

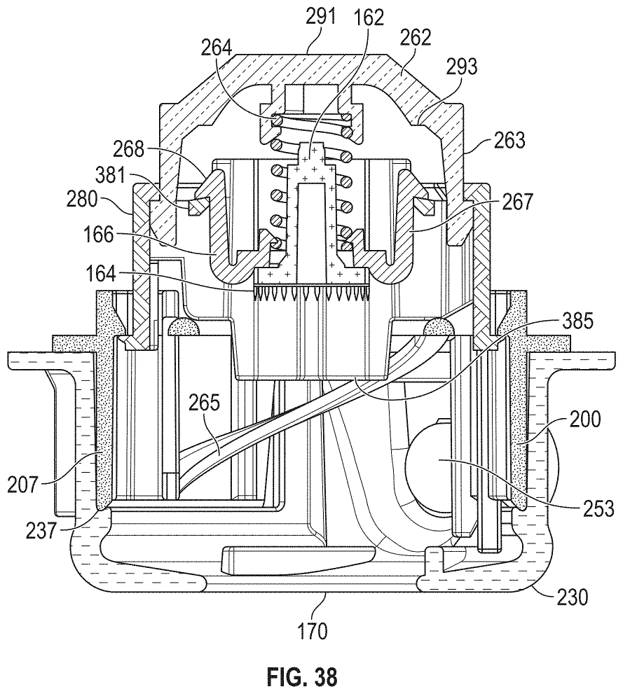

In some embodiments, at least a portion of a wall of the support may overlap with at least a portion of a wall of the interface. In some embodiments, the interface may receive at least a portion of the support, e.g., the support may be at least partially nested within the interface. In the illustrative embodiment of FIG. 38, a portion of the wall 203 of the support 200 overlaps with a portion of the wall 234 of the interface 230.

As seen in the cross-section view of FIG. 38, the interface 230 may include a circumferential groove 237 that receives a circumferential ridge 207 of the support 200 to interlock the support and interface together. This interlock may help to prevent the support 200 from sliding vertically relative to the interface, and/or may be held in a position that allows the lower region of the interface 230 to flex.

The guide housing 280, deployment spring 264, needles 164, latch 266 and push cap 262 may be arranged in a “floating” manner in which they move together as one unit in a deployment direction during device actuation toward the device opening relative to the support ring 200 and the interface 230 until the guide housing 280 meets the user's skin. Contact of the guide housing 280 with skin may trigger the deployment spring 264 to decompress and deploy the needles 164 to pierce skin.

In one illustrative embodiment, the sequence of actuation of the piercing assembly is as follows. As discussed above, the piercing assembly may be physically located under a vacuum source, such as a flexible dome. The flexible dome is compressed, either by a user directly contacting the flexible dome, or by a user interacting with an actuation button, such as the device actuator in the embodiment of FIG. 18. In turn, the inner surface of the flexible dome contacts the push surface 291 of the push cap 262. Force is transmitted to both the deployment spring 264 and the retraction spring 265, causing both springs to compress. Because the retraction spring 265 is less stiff than the deployment spring 264, the retraction spring 265 compresses a greater distance than the deployment spring 264. Compression of the retraction spring 265 allows the guide housing 280, latch 266, push cap 262, deployment spring 264, post 162, and needles 164 to move together in a deployment direction toward the device opening 170 until a bottom surface 385 of the guide housing 280 contacts the subject's skin.

With the guide housing 280 in contact with the subject's skin, the deployment spring 264 continues to undergo compression as user-applied force continues to be applied to the flexible dome and, in turn, the push cap 262. In some embodiments, the push cap may act as a latch release. The push cap 262 moves toward the latch 266, compressing the spring 264, until the contact surfaces 293 of the push cap 262 contacts the cam surfaces 268 of the latch arms 267, pushing the latch arms 267 radially inward until the latch arms 267 clear the ledges 381 of the guide housing 280. When the latch arms 267 clear the ledges 381, the latch 266 is permitted to move in a deployment direction toward device opening 170, thus permitting the deployment spring 264 to decompress. The post 162 and needles 164 are attached to the spring 264. Decompression of the deployment spring 264 causes the needles 164 to move in a deployment direction toward the device opening 170, piercing the user's skin. In some embodiments, the spring 264 is coupled to the push cap 262. In some embodiments, when the deployment spring 264 decompresses, it extends to a position past its resting length. Thus, the spring may be at a length that is longer than its resting length during piercing of the subject's skin. After piercing the subject's skin, the deployment spring 264 may self-retract to its resting length, thereby moving the needle assembly 60 upwardly away from the opening 170. Retraction of the needles may serve to prevent subsequent inadvertent piercing of the skin.

After needle deployment, the user lets go of the device actuator and/or the flexible dome, and user-applied force on the flexible dome ceases. As a result, the retraction spring 265 is free to decompress, causing the guide housing 280 and the needles 164 to move in a retraction direction away from the device opening 170.

It is recognized that, in some situations, the retraction spring may tend to rotate during compression. For example, in the illustrative example of the retraction spring 265 with cantilevered helical arms, the arms tend to rotate during compression. It is appreciated that, in some situations, it may be desirable to prevent the retraction spring from imparting rotation to other components of the device. As discussed above, in some embodiments, when in a decompressed state, the retraction spring 265 is positioned between the guide housing 280 and a bottom portion of the support 200. In some embodiments, the retraction spring may be unattached to the guide housing 280 such that the two components are free to slide relative to one another. The retraction spring 265 may, however, be attached to the support 200. For example, as the guide housing 280 is moved in the deployment direction toward the device opening during compression of the retraction spring 265, a surface of the guide housing slides against the retraction spring 265. In some cases, this unattached arrangement may help to decrease impartation of rotational movement of the retraction spring 265 to the guide housing 280.

In some embodiments, the device may include one or more features that help to limit certain movement of the retraction spring during compression and/or decompression. In some embodiments, the retraction spring may be shaped such that the arms of the retraction spring may tend to move radially outwardly during compression.

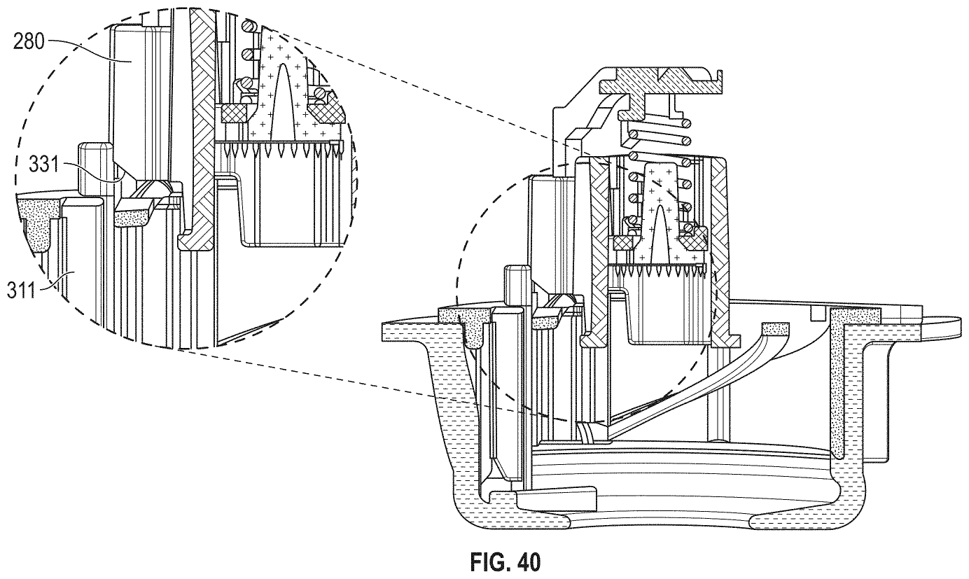

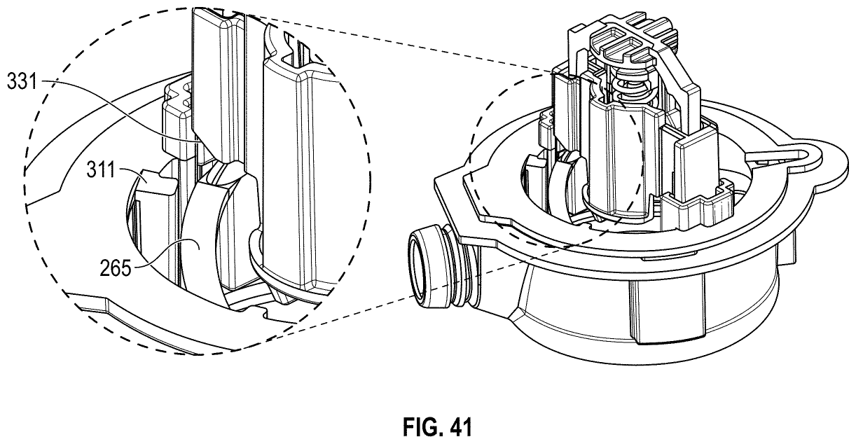

In some embodiments, the guide housing may include one or more features that help to limit certain movement of the retraction spring. In one illustrative embodiment, as shown in FIGS. 40 and 41, the guide housing 280 includes a notch 331 in which the arms of the retraction spring 265 slide during compression and/or decompression of the retraction spring. The notch 331 helps to prevent the arms of the retraction spring 265 from sliding radially outwardly beyond a certain point.

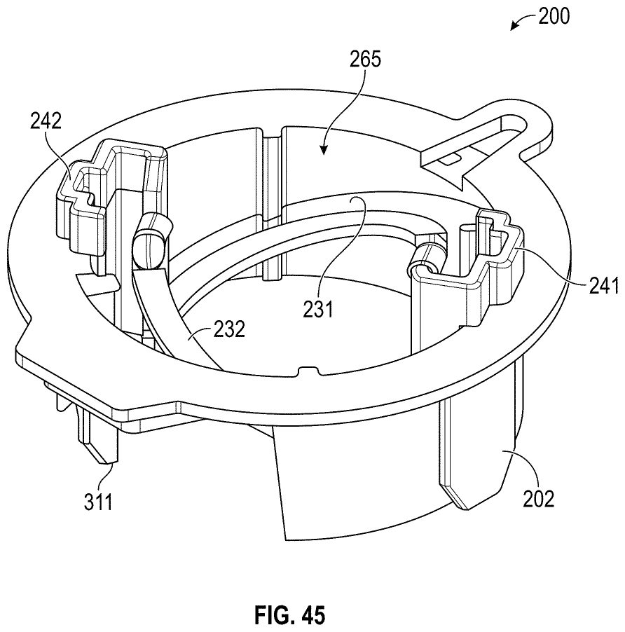

In some embodiments, the support may include one or more features that help to limit certain movement of the retraction spring. In one illustrative embodiment, as shown in FIGS. 40 and 41, the support 200 may include rails 311 against which the arms of the retraction spring 265 slide during compression and/or decompression. The rails 311 may help to prevent the arms of the retraction spring from sliding radially outwardly beyond a certain point. FIGS. 45-47 also show the rails 311.

In some embodiments, the notch on the guide housing and the rails of the support cooperate to limit certain movement of the retraction spring. The arms of the retraction spring may be bounded on one side by the notch and on another side by the rails.

It is recognized that, in some situations, it may be desirable to promote linear movement of needles into and out of skin. According to one aspect, the device may include one or more features that aid in guiding movement of one or more components of the piercing assembly during deployment and/or retraction. In some embodiments, such movement-guiding features may help to promote linear movement, e.g. by limiting rotation and/or tilt of one or more components during deployment and/or retraction.

As discussed above, in some embodiments, the actuation sequence may begin with compression of the retraction spring 265, which allows the guide housing 280, latch 266, push cap 262, deployment spring 264, post 162, and needles 164 to move together in a deployment direction toward the device opening 170. In some embodiments, this deployment direction movement may be guided by interaction between guide features on the guide housing 280 and corresponding guide features on the support 200. In one illustrative embodiment, the guide features are in the form of tracks 241, 242 on the support 200 and wings 281, 282 on the guide housing 280. The tracks 241, 242 are shaped to receive the wings 281, 282 and to guide linear movement of the guide housing 280, e.g. during compression and/or decompression of the retraction spring 265. The tracks 241, 242 of the support may help to constrain the guide housing from rotating and/or tilting during movement.

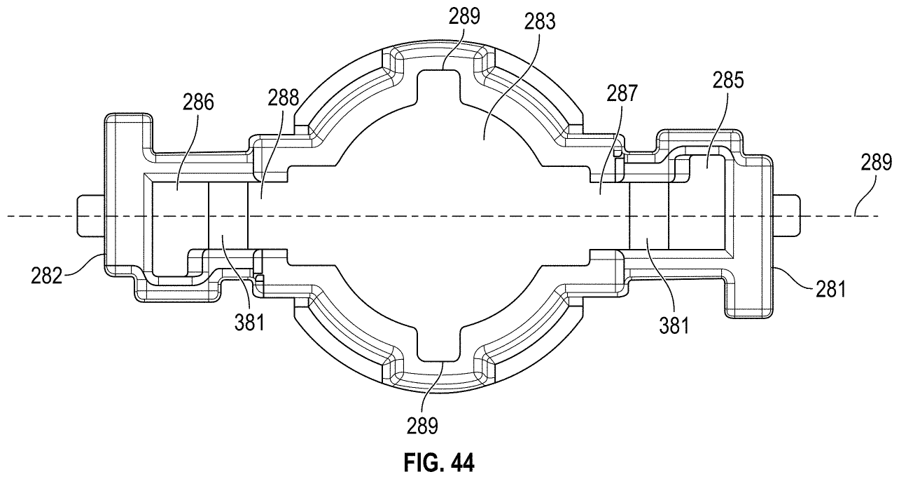

As discussed above, in some embodiments, after the guide housing 280 reaches a user's skin, the push cap 262 may move toward the latch 266, and the deployment spring 264 may compress. In some embodiments, the device includes guide features that guide movement of the push cap 262. In one illustrative embodiment, these guide features are in the form of tracks on the guide housing. As seen in the top view of the guide housing 280 in FIG. 44, in some embodiments, the guide housing 280 may include push cap tracks 285, 286 that receive arms 263 of the push cap 262. The tracks 285, 286 guide linear movement of the push cap, e.g. during compression and/or decompression of the deployment spring 264. The tracks 285, 286 may help to constrain the push cap 262 from rotating and/or tilting during movement.

In some embodiments the device may include guide features that help to guide movement of the latch. In one illustrative embodiment, these guide features are in the form of tracks on the guide housing. As seen in the top view of the guide housing 280 in FIG. 44, in some embodiments, the guide housing 280 may include latch tracks 287, 288 that receive the arms 367 of the latch 266. The tracks 287, 288 guide linear movement of the latch, e.g. during decompression and/or extension of the deployment spring 264. In some embodiments, additional guide features may be provided in the form of tracks 289 on the guide housing that receive additional arms 369 of the latch 266. The tracks 289 may further help to guide linear movement of the latch 266.

In some embodiments, the needle assembly, which may include one or more needles, may be attached to the latch 266 such that movement of the latch 266 moves the plurality of needles. In one illustrative embodiment, the needle(s) are attached to the latch via a post 162. In some embodiments, the needle(s) move through the guide housing 280 during deployment and/or retraction. As seen in the top view of the guide housing 280 in FIG. 44, the guide housing may include an opening 283 through which the post 162 and/or the needle(s) may move.

It should be appreciated that, in some embodiments, the guide features described above do not completely eliminate all rotation and/or tilting of components, but rather may serve to limit the amount of rotation and/or tilting of the components.

As discussed above, in some embodiments, the retraction spring may be a pair of cantilevered helical arms. In some embodiments, the retraction spring is integrated with the support such that the support and retraction spring form a single, monolithic component (e.g. molded as one-piece). In other embodiments, however, the retraction spring and support are formed as separate pieces, and are later brought together during assembly.

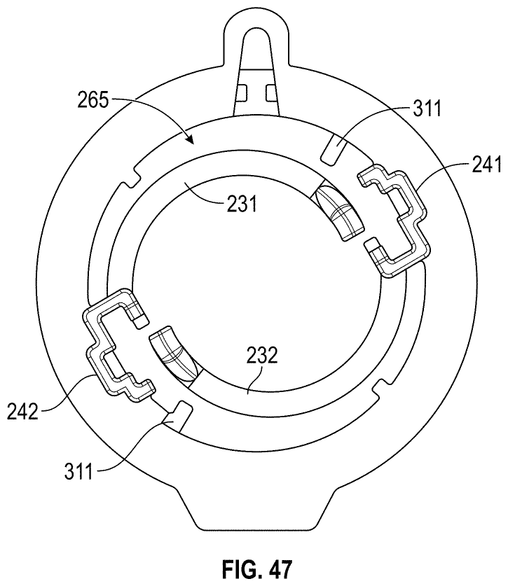

As seen in FIGS. 45-47, the retraction spring 265 includes two arms 231, 232 that are cantilevered helical arms that are part of the support 200. As seen in FIG. 47, the arms extend from the tracks 241, 242.

Because the flexible dome 140 is biased to return to its original, non-compressed shape, when the user stops pushing on the device actuator, in some embodiments, without the ratchet, the flexible dome 140 would revert back to its original shape, pressing up on and moving the device actuator upward. If a user were to prematurely release the device actuator prior to complete actuation (e.g., letting go of the device actuator prior to pressing the device actuator all the way down until the device actuator bottoms out), the flexible dome may not have been fully compressed, and thus may not generate as much vacuum as it would have compared to a full compression of the flexible dome. Thus, in preventing the device actuator 171 from moving in the return direction upward, the teeth of the ratchet may help to prevent insufficient generation of vacuum. In addition, because the device actuator remains in a fixed position due to the ratchet holding the device actuator in place, the ratchet may provide a visual indicator to a user that actuation is incomplete.

However, it should be appreciated that, in some embodiments, the flexible dome is used in a device without a ratchet. As an example, the ratchet may be removed from the

According to one aspect, the flexible dome may include one or more features that promote its performance. A flexible dome may be designed to be biased to return to an original configuration after being compressed. The force that urges the flexible dome to return to its original configuration is referred to herein as the flexible dome's return force. It is appreciated that the vacuum generated by the flexible dome may resist return of the flexible dome back to its original shape (e.g. if the force due to the vacuum is greater than the return force), which may in turn limit the amount of vacuum that can be generated by the flexible dome. The potential need is recognized, in some embodiments, for a feature that helps to promote return of the flexible dome back towards its original shape.

It should be noted that it is recognized that a flexible dome need not fully return to its original configuration to produce a sufficient amount of vacuum.

It is also recognized that repeatedly returning to or near to the original configuration

The technical challenges of using a flexible dome that is designed to be manually compressed to generate vacuum is recognized. On the one hand, it is appreciated that, in some cases, a chamber that is easy for a user to compress may not return to a sufficient enough degree to produce sufficient vacuum (e.g. may not have a shape/design/material properties that gives rise to a strong return force), while on the other hand, a chamber that will return fully or near to fully may be too difficult for a user to compress fully. Without wishing to be bound by theory, it is recognized that, in some cases, a stiff dome will have a strong return force but is difficult to fully compress, while a soft dome is easy to fully compress, but has a weak return force (e.g. will have difficulty returning to its original volume after being compressed). In some embodiments, the flexible dome may be configured to return to a predefined position. It is recognized that such a dome may provide increased control over the level of vacuum produced.

Some embodiments described herein are directed to providing a flexible dome that is harder to compress during an initial phase of compression, and easier to compress during the final phase of compression. This may result in a return force that is lower during an initial phase of return (when the force resisting return is lower, e.g. because the magnitude of vacuum generated at this point is low) and a return force that is higher during the final phase of return (when the force resisting return is higher, e.g. due to the high magnitude of vacuum generated).

In some embodiments, the flexible dome may be arranged to be stiffer during an initial phase of compression, and more compliant during the final phase of compression. In some embodiments, the dome includes a feature that promotes buckling of the dome as the dome is compressed, resulting in a dome that is stiffer during an initial phase of compression, and more compliant during the final phase of compression. Conversely, the return force may be lower during an initial phase of return and higher during the final phase of return.

In some embodiments, the flexible dome includes an indented shoulder that helps to promote return movement of the flexible dome back towards its original shape. The indented shoulder may promote buckling of the dome as the dome is compressed. It should be appreciated that, in some embodiments, movement of the flexible dome back towards its original shape does not necessarily mean that the flexible dome actually reaches a full return to its original, non-compressed shape. In some embodiments, complete return of the flexible dome back to its original, non-compressed shape is not achieved and is not required.

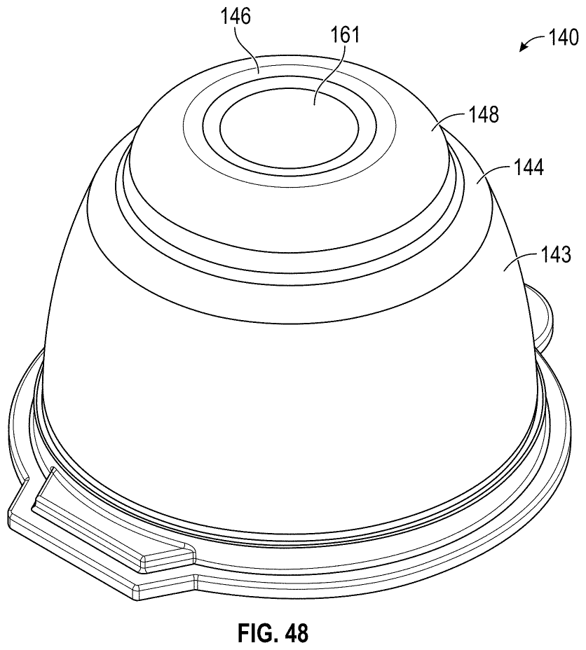

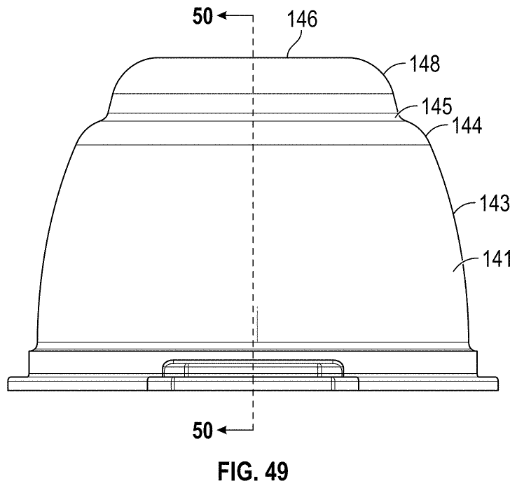

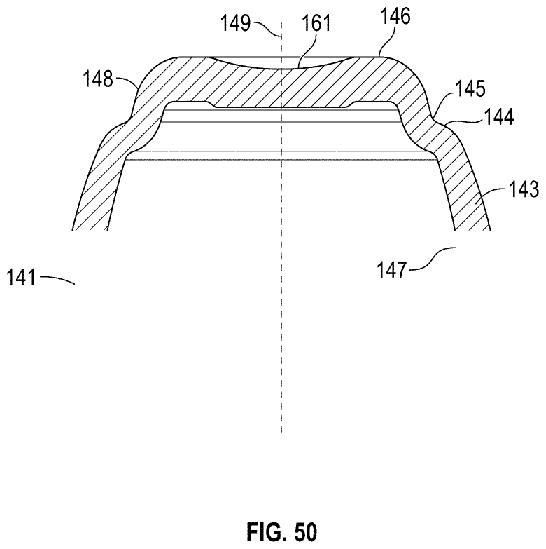

A perspective view of a flexible dome 140 is shown in FIG. 48. The dome has a wall 143 having a lower portion 141 and an upper portion 148. The wall 143 includes an indented circumferential shoulder 144 between the upper and lower portions. The shoulder 144 may create a change in concavity direction of the wall 143. As seen in the side view of FIG. 49 and the cross-section view of FIG. 50, the lower portion 141 of the wall 143 may be curved such that a concavity 147 of the lower portion 141 faces inwardly toward a longitudinal axis 149 of the dome. At the region of the shoulder 144, however, the concavity 145 of the wall may face outwardly away from a longitudinal axis 149 of the dome.

In some embodiments, the shoulder may be positioned at a height on the dome that is in the top-half height of the dome. In some embodiments, the shoulder may be positioned at a height on the dome that is in the top-third, or top-quarter of the height of the dome. In some embodiments, positioning the indented shoulder in an upper half of the dome may cause the flexible dome to be arranged to be stiffer during an initial phase of compression, and more compliant during the final phase of compression.

In some embodiments, the top of the flexible dome may include an indentation. As seen in FIGS. 48 and 50, the flexible dome 140 includes a circular indentation 161 at the top 146 of the dome.

In some embodiments, the thickness of the wall of the flexible dome may vary along different heights of the dome. In the illustrative embodiment of FIG. 50, the lower portion 141 of the wall becomes thicker as it moves from the shoulder 144 down away from the top 146 of the dome. In alternative embodiments, however, the wall thickness remains constant.

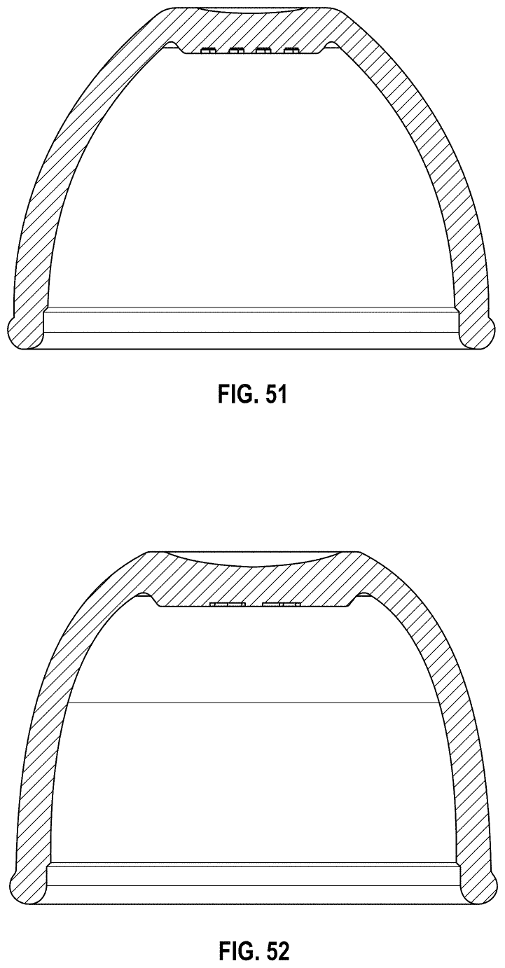

In some embodiments, the flexible dome does not include a shoulder that changes the concavity direction of the wall. Alternative shapes for the flexible dome are shown in FIGS. 51 and 52, each of which do not include an indented shoulder. The dome shapes shown in FIGS. 51 and 52 each include an indentation at the top of the domes. However, in other embodiments, this indentation may be omitted.

In some cases, the flexible domes described can be used for generating vacuum to assist in receiving fluids or other materials, such as blood or interstitial fluid, from subjects, e.g., from the skin and/or beneath the skin. However, the flexible domes described are not limited to only such applications. In other cases, the flexible domes as are described herein may be used in any application where a vacuum is desired to be created. Examples include, for example, priming of oil pumps, creation of suctions to attach objects together, or movement of fluids or other substances from one location to another location.

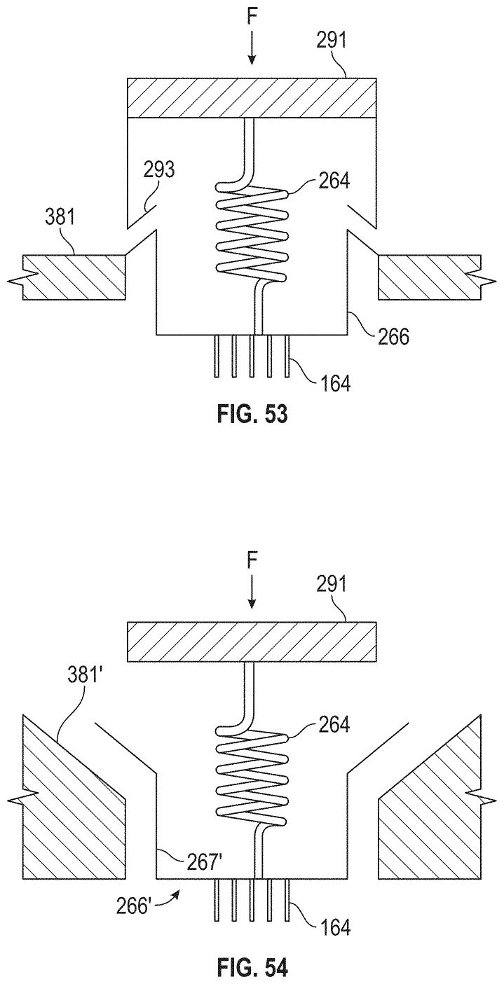

In some embodiments, deployment of the needles is triggered via a latch release that releases a deployment spring from a compressed state to drive deployment of the needles. In some embodiments, such as in the illustrative embodiment of FIG. 38, the latch release is a distance-based latch release, in that the latch release must travel a pre-defined distance to reach and release a latch that holds the spring in its compressed state. A schematic illustration of a distance-based latch release is shown in FIG. 53. Contact surfaces 293 must travel a pre-defined distance to reach and release latch 266. The deployment spring 264 is positioned between the contact surface 293 (or the device actuator itself, or other component coupled to the device actuator) and the latch 266. During movement of the contact surface 293 toward the latch 266 (or otherwise during movement of the device actuator), the deployment spring 264 is compressed.

In alternative embodiments, the latch release is a force-based latch release, in that a threshold actuation force must be applied to release the latch, rather than requiring a minimum travel distance. A schematic illustration of a force-based latch release is shown in FIG. 54. As an actuation force F is applied to the push surface 291 (and/or device actuator), the latch 266 does not clear the ledge 381′ until the actuation force exceeds a threshold force. The threshold force may be determined by multiple factors. In some embodiments, the arrangement includes a ledge 381′ having a sloped contact surface that is in contact with the latch 266′ during application of an actuation force. In some embodiments, one factor affecting the threshold force may include the angle of the sloped contact surface. E.g. a steeper angle may lower the threshold force and a flatter angle may increase the threshold force. In some embodiments, one factor affecting the threshold force may include the amount of friction between the latch 266′ and the ledge 381′. An increase in the amount of friction may increase the threshold force. In some embodiments, one factor affecting the threshold force is the stiffness of the cantilevered arms 267′ of the latch 266′. The deployment spring 264 is coupled to move with the push surface 291 (and/or device actuator). The spring 264 begins to compress when actuation force F is applied.

According to one aspect, the spring stiffness and the latch characteristics affecting the threshold force are balanced with one another to control the behavior of the spring. The spring stiffness and threshold force may determine the amount of compression that the stiff undergoes prior to unlatching. In some embodiments, the spring stiffness and threshold force for unlatching are chosen to ensure that the spring extends a set distance beyond its resting length. For a given spring having a given stiffness, the latch may be tuned to release at a threshold force that is equal to or greater than the stiffness of the spring multiplied by the desired spring extension distance. Or, for a latch having a given threshold release force, a spring stiffness may be chosen such that the threshold force is equal to or greater than the stiffness of the spring multiplied by the desired spring extension distance.

In some embodiments, the device includes a deployment actuator that moves the needles in a deployment direction toward the opening, and a retraction actuator that moves the needles in the opposite direction: a retraction direction away from the opening. In some embodiments, each of the deployment actuator and the retraction actuator act as springs that can be manipulated to store potential energy, and release of the stored potential energy drives movement of the needles.

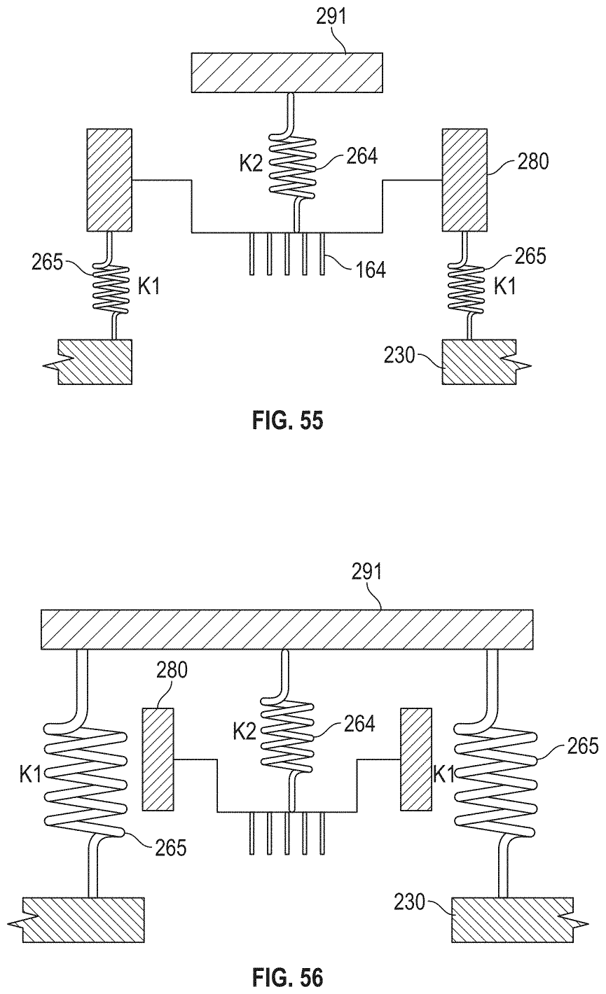

In some embodiments, such as in the illustrative embodiment of FIG. 38, the deployment actuator and retraction actuator are arranged as springs in series. A schematic illustration is shown in FIG. 55. The deployment spring 264 is coupled to the needle assembly 164 such that the needle assembly 164 moves with movement of the spring 264. The deployment spring 264 is positioned between the push surface 291 (and/or device actuator) and the guide housing 280 such that movement of the push surface 291 (and/or device actuator) relative to the guide housing 280 toward the interface 230 of the device compresses the deployment spring 264. The retraction spring 265 is positioned between the guide housing 280 and the interface 230 such that movement of the guide housing 280 toward the interface 230 compresses the retraction spring 265. Release of the retraction spring 265 from a compressed state moves the guide housing 280 in a retraction direction away from the interface 230, which in turn moves the needle assembly 164 in a retraction direction. The deployment spring 264 has a stiffness K2 and the retraction spring 265 has a stiffness K1. With the deployment spring and retraction spring arranged in series, the springs compress simultaneously. In some embodiments, the stiffness K1 of the retraction spring 265 may be lower than the stiffness K2 of the deployment spring 264 to permit the retraction spring to compress to a target distance before the deployment spring compresses to its target release distance.

In alternative embodiments, the deployment actuator and retraction actuator are arranged as springs in parallel. A schematic illustration is shown in FIG. 56. Similar to the springs in series arrangement, the deployment spring 264 is positioned between the push surface 291 (and/or device actuator) and the guide housing 280 such that movement of the push surface 291 (and/or device actuator) relative to the guide housing 280 toward the interface 230 of the device compresses the deployment spring 264. However, in contrast to the springs in series arrangement, the retraction spring 265 is positioned between the push surface 291 (and/or device actuator) and the interface 230 rather than between the guide housing 280 and the interface 230. As such, movement of the push surface 291 (and/or device actuator) toward the interface 230 compresses the retraction spring 265 in addition to compression of the deployment spring 264. In such an arrangement, the stiffness of the springs may be independent of one another. In some embodiments, the deployment spring only begins compressing once a reaction force is provided via the skin or a component in the device.

It should be appreciated that the device may be actuated by various different actions/gestures. In some embodiments, the device may be actuated by squeezing, twisting, pulling, pressing, pinching, spinning, or by any other suitable action.

In some embodiments, a user may pull back on a spring-loaded rod and release, or otherwise actuate the device to cause a spring-loaded rod to be pulled back and released, causing needles to deploy into skin. In some embodiments, a user could cock a deployment mechanism to place a device in a ready to actuate state. E.g., a user could compress or pull back on a spring until it reaches a latched state, and then actuate a device actuator to release the cocked spring. In other embodiments, the spring may be assembled in a pre-compressed or pre-elongated state prior to any user interaction with the device, and the user may release the spring by, e.g., actuating a device actuator.

In illustrative embodiments above, the deployment actuator and the retraction actuator are springs. However, it should be appreciated that other arrangements for the deployment actuator and/or the retraction actuator are possible. For example, the deployment actuator and the retraction actuator may each include any number of suitable components, such as a button, a switch, a lever, a slider, a dial, a compression spring, a Belleville spring, compressible foam, a snap dome, a servo, rotary or linear electric motor, and/or a pneumatic apparatus, or other suitable device. Also, the deployment actuator and the retraction actuator may be of the same type, or may be different types of devices. Each actuator may operate manually, mechanically, electrically, pneumatically, electromagnetically, or other suitable mode of operation, and may or may not require user input for activation.

In some embodiments, the flexible dome may serve as a retraction actuator that retracts the needle assembly away from the opening. As such, the flexible dome may both generate vacuum and retract the needle assembly. The needle assembly may be coupled to the flexible dome such that, as the flexible dome reverts from a compressed state back toward its original uncompressed state, the needle assembly is pulled up in a retraction direction away from the opening with the dome. In some embodiments, the needle assembly may be coupled to an inner surface of a top portion of the dome. For example, the surface 291 may be attached to the flexible dome, e.g. by adhesive, UV welding, mechanical interlock, or any other suitable attachment method.

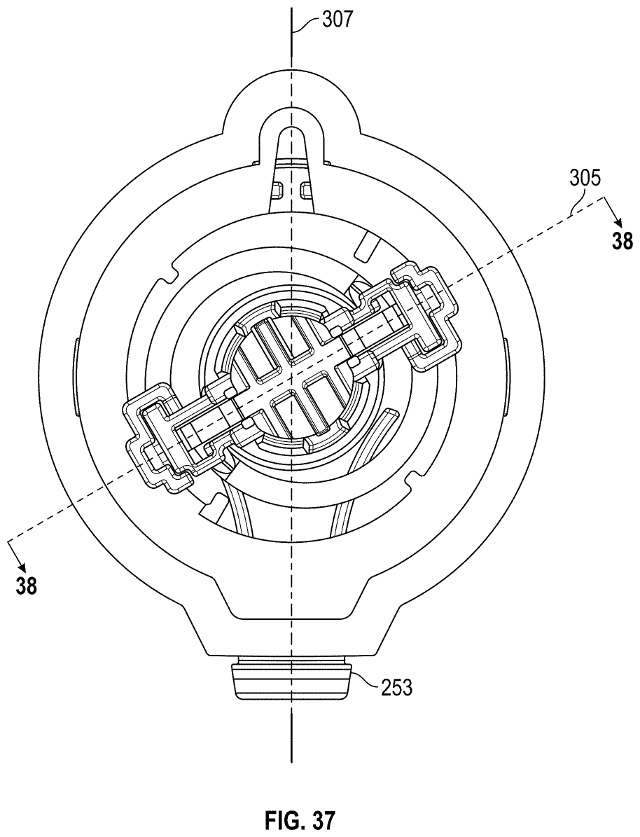

According to one aspect, the components of the piercing assembly are positioned to permit flow of fluid into an outlet that may lead to a storage container. As seen in FIG. 38, the helical arms of the retraction spring 265 are in a position that do not obstruct the outlet 253. In one illustrative embodiment, as seen in FIG. 37, the piercing assembly has an angular relationship relative to the outlet 253. A longitudinal axis 305 of the guide housing is at an angle relative to a longitudinal axis 307 of the outlet 253. In this illustrative embodiment, the angular relationship of the piercing assembly relative to the outlet places the arms of the retraction spring 265 in a position that does not obstruct the outlet 253.

It should be appreciated that the device is not limited to the piercing assemblies depicted in the figures. For example, in one embodiment, the piercing assembly comprises one or more needles coupled to the inside of an elastic dome. A user may push down on the dome to push the needles into skin, and the dome may be biased to spring back to its initial position, thereby retracting the needles from the skin.

In another embodiment, the piercing assembly comprises a snap dome combined with an elastic dome, where one or more needles are coupled to the snap dome. A user may compress the elastic dome to bring the needle(s) and snap dome close to the skin, the snap dome may be actuated to invert to deploy the needle(s) into the skin, and the elastic dome may be biased to spring back to its initial position, thereby retracting the needles from the skin.

In some embodiments, the piercing assembly includes an elastic dome having strategic imperfections that control the buckling behavior of the elastic dome. For example, a user may apply a force to the dome, the dome may eventually buckle due to the imperfections, driving the needle(s) into skin, and the dome may spring back to its initial position after removal of the user's applied force.

In some embodiments, the piercing assembly may have an elastic dome with deployment and/or retraction behavior that is tailored by the use of non-uniform material properties or co-molding. For example, in some embodiments, the piercing assembly comprises a snap dome molded into an elastic dome.

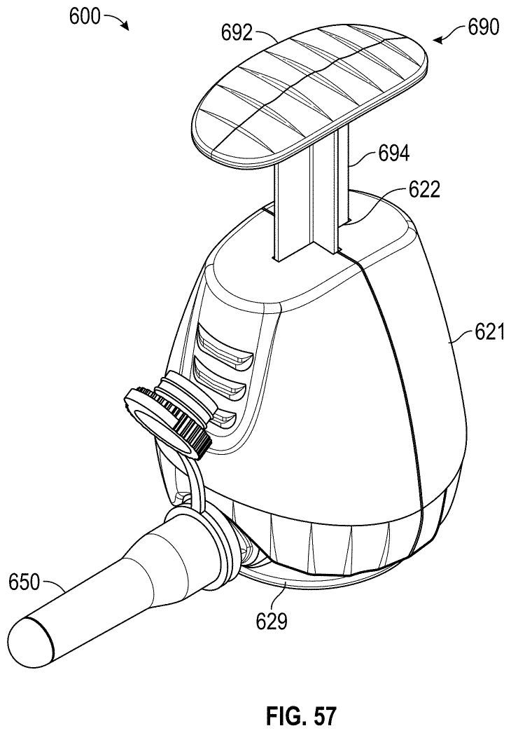

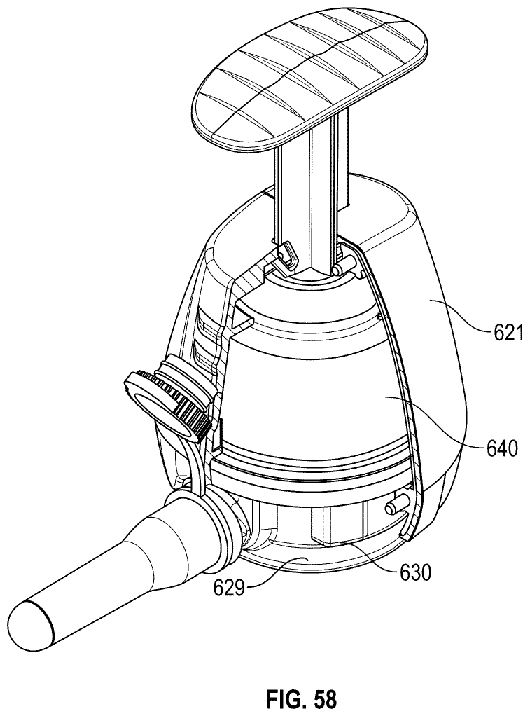

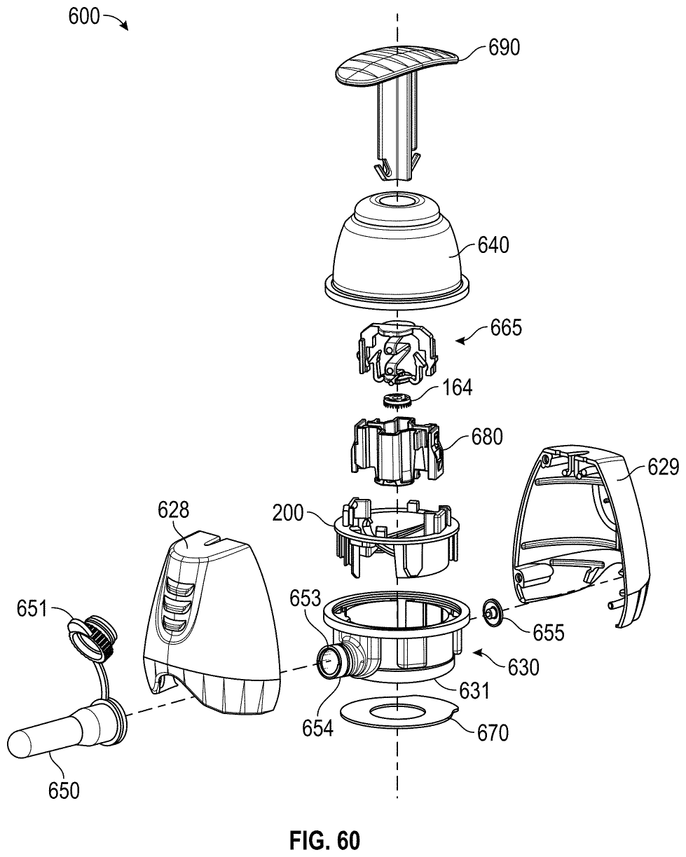

Another illustrative embodiment of a fluid receiving device 600 is shown in FIG. 57, and the components of the fluid receiving device 600 are shown in the exploded view of FIG. 60. The device 600 may be largely similar to the earlier embodiment of FIG. 18. For example, the device may have a device actuator 690 that is distinct from a vacuum source 640, which may also be in the form of a flexible dome, as shown in the partial cutaway view of FIG. 58. The device actuator 690 may have a user-contacting portion 692 and a stem 694. The device may include a housing 621 having an opening 622 through which the stem 694 of the device actuator 690 can move. Fluid received by the device may be stored in storage chamber 650. In some embodiments, the device 600 does not include a ratchet arrangement. However, in other embodiments, the device may include any of the ratchet arrangements described above in earlier embodiments.

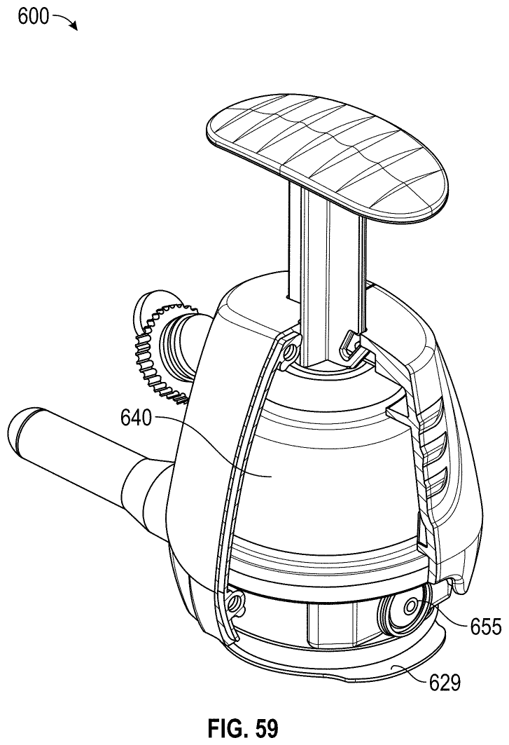

As shown in FIGS. 59 and 60, the device may include a one-way valve 655 that may be the form of an umbrella valve.

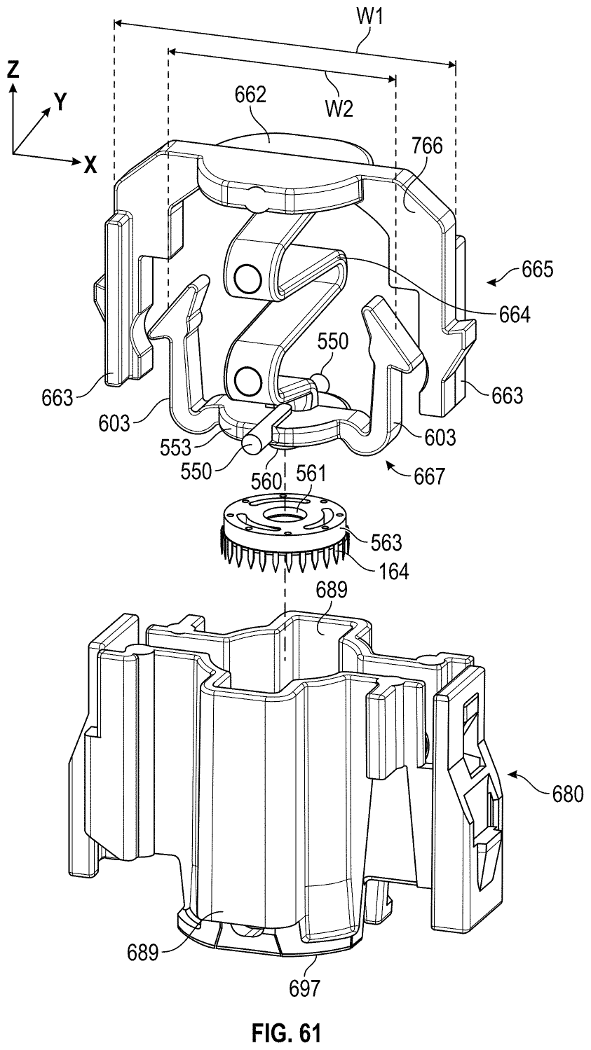

As shown in the illustrative embodiment of FIGS. 60 and 61, in some embodiments, a fluid receiving device may include a latch and spring assembly 665 including a latch release 766, a spring 664, and a latch 667. In some embodiments, the entire latch and spring assembly 665 may be integrally formed as a single piece. In some embodiments, a subset of the latch and spring assembly are integrally formed as single piece (e.g. the spring 664 may be integrally formed with the latch release 766, or the spring 664 may be integrally formed with the latch 667), and the remaining component is later attached. Any combination of these manufacturing arrangements may be used as well, e.g. two of the three components are integrally formed with one another, and the third is later attached.

As used herein, parts that are “integrally formed” with one another means that the parts are formed as one component such that they are formed from a single monolithic component, e.g., cast at the same time as a single piece such as in die casting or injection molding, or cut from a single material such as in stamping or die cutting.

The latch and spring assembly may be manufactured via injection molding, stamping, die casting, die cutting, or via any other suitable manufacturing process.

The latch and spring assembly 665 may be coupled to the needles 164 in any suitable manner. In the illustrative embodiment of FIG. 61, the needles 164 may be mounted to a base 563, which may attach to the latch 667 and/or the spring 664 of the latch and spring assembly 665. In some embodiments, a post 560 attached to the latch 667 and/or the spring 664 of the latch and spring assembly 665 may pass through a hole 561 of the base 563. The post 560 may be attached to the base 563 by heat-staking, welding, adhesive, interference fit, snap fit, mechanical interlock, mechanical fasteners, or any other suitable attachment method, and any combination of the above.

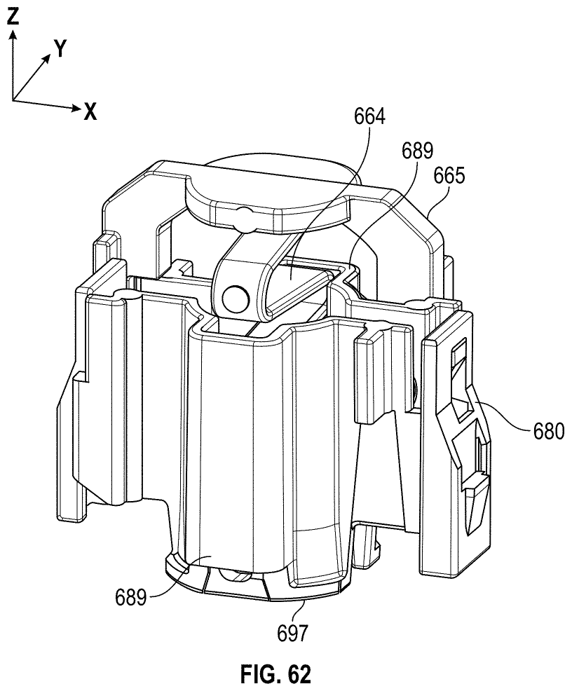

Similar to earlier described embodiments, the device 600 may include a guide housing 680 that may help to guide movement of the latch and spring assembly 665 during needle deployment and/or retraction. FIG. 62 shows the latch and spring assembly 665 received by the guide housing 680 in a state prior to spring compression and prior to needle deployment. The guide housing 680 may include tracks 689 that are sized and positioned to receive the spring 664 to guide linear movement of the spring 664 as the spring compresses and decompresses, thereby guiding linear movement of the needles 164 that are coupled to the spring 664.

According to one aspect, in some embodiments, the fluid receiving device includes a positive stop that is configured to limit needle distance relative to the device's interface with the skin. It is recognized that, in some situations, it may be beneficial to limit the travel distance of the needles in order to control the insertion depth of the needles into skin. However, it should be appreciated that a positive stop is not necessary in all embodiments of the device.

In some embodiments, a positive stop that limits needle insertion is formed via an interaction between the latch and spring assembly with the guide housing. The positive stop may limit the travel of the penetrating end of the needles relative to a reference point on the device, such as a distal surface of the guide housing.

As shown in FIG. 61, in some embodiments, the piercing assembly may include one or more pegs 550 that contact a corresponding surface on the guide housing during spring decompression to limit the movement distance of the needles. While two pegs are shown in the illustrative embodiment of FIG. 61, it should be appreciated that any number of pegs may be used, such as, but not limited to, 3, 4, 5, 6, 7 or 8 pegs.

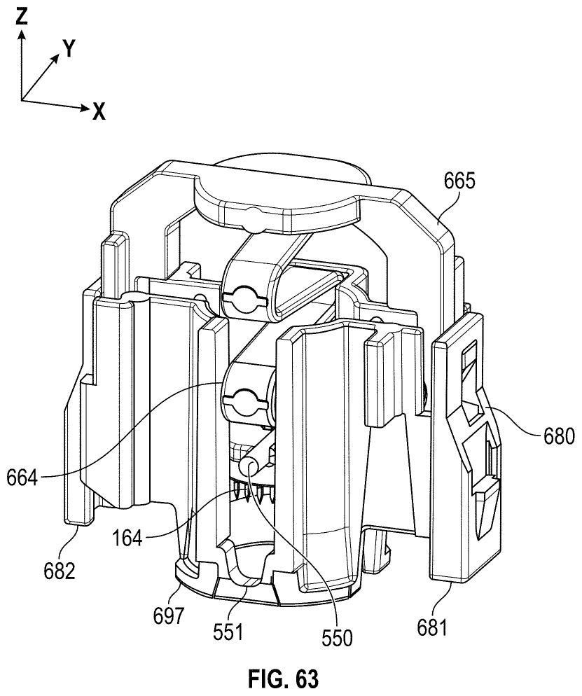

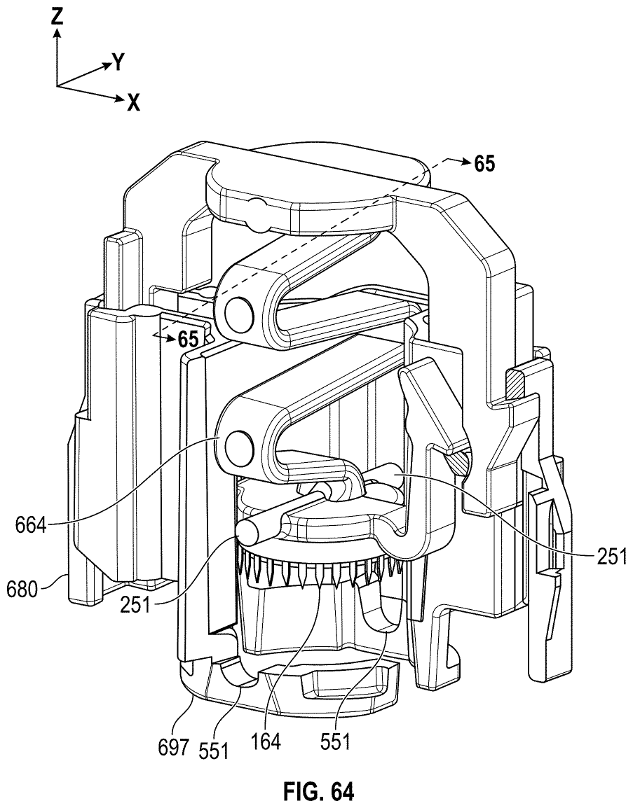

As shown in FIGS. 63 and 64, in which portions of the guide housing 680 are hidden, the guide housing may include contact surfaces 551 that are positioned to make contact with the pegs 550 as the spring 664 decompresses, and as the needles 164 move in a deployment direction. Contact between the pegs 550 and the contact surfaces 551 creates a positive stop that prohibits the needles 164 from further distal movement, even if the spring 664 has not yet completely decompressed. In some embodiments, this positive stop may limit the travel distance of the distal end of the needles 164 beyond a distal surface 697 on the guide housing 680.

In some embodiments, the travel distance of the distal end of the needles beyond a distal surface on the guide housing is limited to less than or equal to 6, 5, 4, 3, 2, 1, 0.9, 0.8, 0.7, 0.6, 0.5, or 0.4 mm. In some embodiments, the travel distance of the distal end of the needles beyond a distal surface on the guide housing is limited to at least 0.1, 0.2, 0.3, 0.4, 0.5, 0.6, 0.7, 0.8, 0.9, 1, 2, 3, 4 or 5 mm. It should be appreciated that combinations of the above-referenced ranges are also possible. For example, in some embodiments, the travel distance of the distal end of the needles beyond a distal surface on the guide housing is limited to 0.1 to 6, 0.5 to 5, 0.5 to 4, or 0.5 to 3 mm.

The pegs 550 may be formed on and/or coupled to any suitable portion of the latch and spring assembly 665, such as the latch 667, the spring 664, the needle base 563, or the post 560 (see FIG. 61). In the illustrative embodiment of FIG. 61, the pegs 550 extend from a hub 553, where the hub 533 connects the latch arms 603, and may also connect the spring 664 to the latch 667.

In the illustrative embodiment of FIGS. 63 and 64, the contact surfaces 551 are U-shaped. In other embodiments, the contact surfaces may be flat, convex, or any other suitable shape.

It should be appreciated that the positions of the pegs and contact surfaces may be reversed such that the pegs are on the guide housing and the contact surfaces are on the latch and spring assembly, or on the needle base.

In other embodiments, the contact surfaces may be on components other than the guide housing. For example, the contact surfaces may be on the support ring 200, the housing 621, or the interface 630.

In some embodiments, a positive stop may be created by interaction between the latch release 766 and the latch 667. During spring decompression, a surface on the latch may contact and abut against a surface on the latch release, and may limit decompression of the spring, and as a result, restrict travel distance of the needles.

In some embodiments, a positive stop may be created by a primary interaction between the latch and spring assembly and the latch release, and a secondary interaction between the latch release and the guide housing.

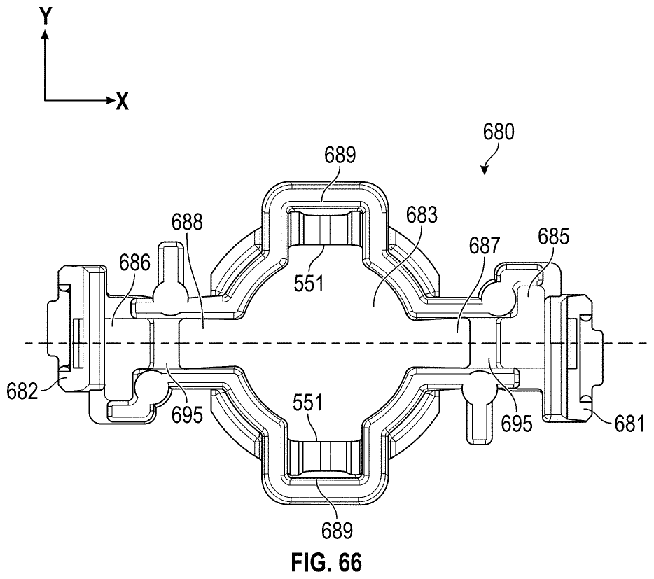

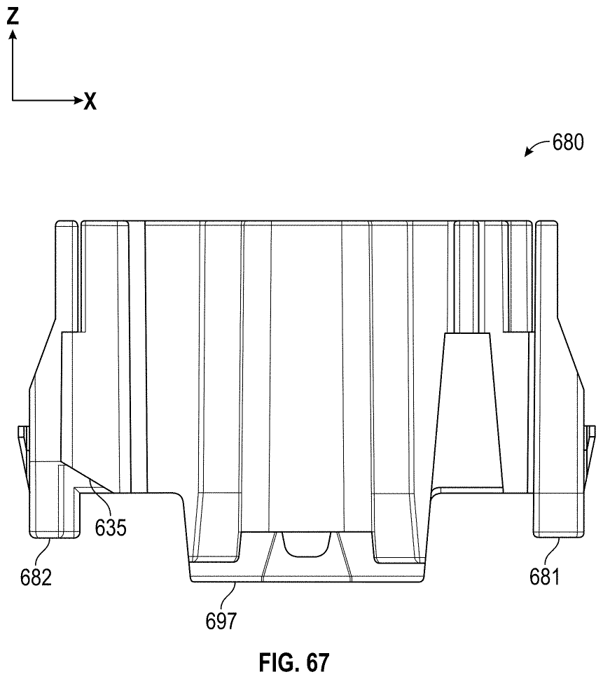

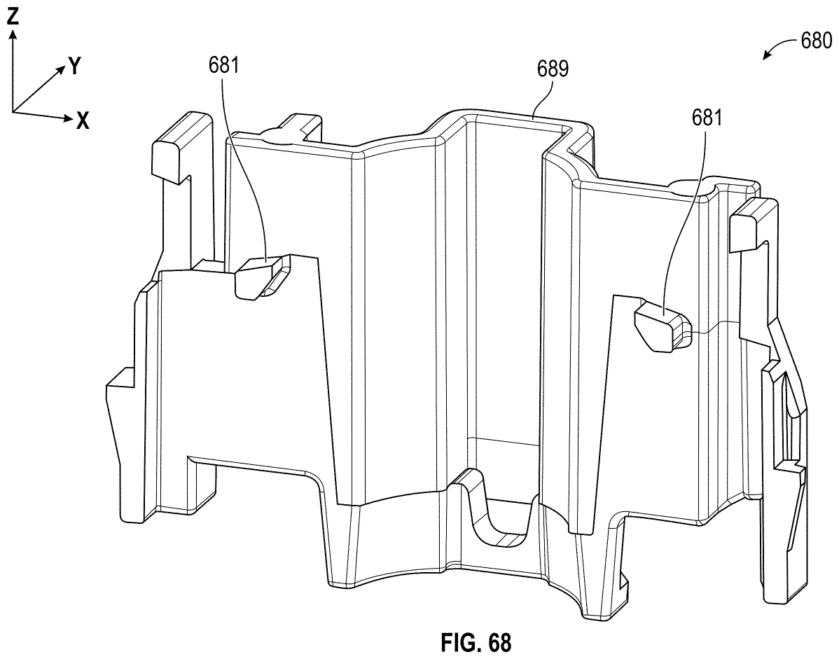

Similar to earlier embodiments, as shown in the top view of FIG. 66, the guide housing 680 may also include tracks 685 that receive and guide linear movement of the arms 663 of the latch release 766. The guide housing 680 may also include latch tracks 687 that receive and guide linear movement of the arms of the latch 667. Also similar to earlier embodiments, as shown in FIG. 66 and the partial cutaway view of FIG. 68, guide housing 680 may include ledges 695 that the arms of the latch 667 initially engage with prior to device actuation. Also similar to earlier embodiments, the guide housing 680 may include wings 681, 682 that are received by tracks on the support 200 to help constrain the guide housing from rotating and/or tilting during movement. Also similar to earlier embodiments, as seen in FIG. 67, the guide housing 680 may include a notch 635 that may help to prevent the arms of the retraction spring 265 (see FIG. 45) of the support 200 from sliding radially outwardly beyond a certain point.

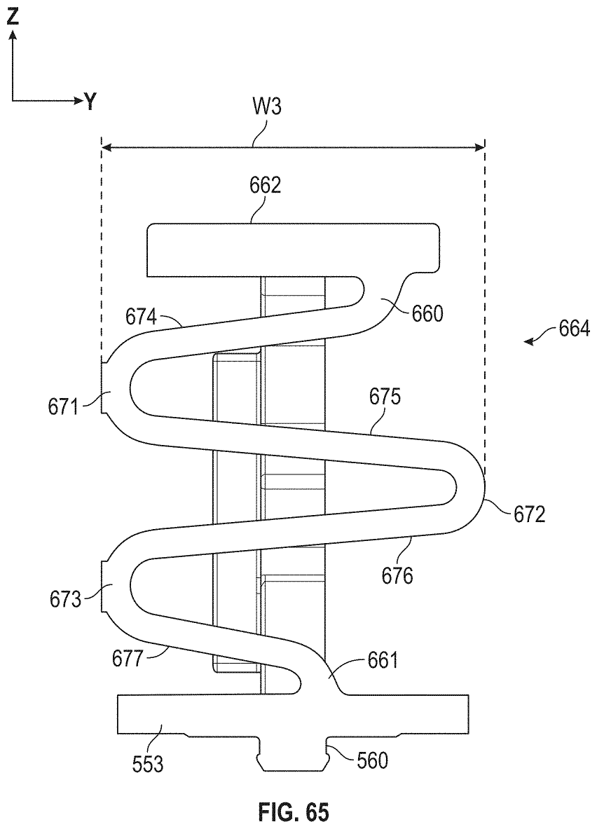

As shown in the embodiment of FIGS. 60-65, in some embodiments, a spring need not be a coil spring. In the illustrative embodiment of FIGS. 60-65, the spring 664 has an undulating shape (e.g. in a back and forth manner that is a non-coil shape). A cross-section of the spring 664 taken along line 65-65 of FIG. 64 is shown in FIG. 65. The spring 664 has a first end 660 at the cap 662 and terminates at a second end 661 at the hub 553. In the illustrative embodiment of FIG. 65, the spring has a first beam 674, a second beam 675, a third beam 676, and a fourth beam 677. A first curve 671 joins the first beam 674 to the second beam 675, a second curve 672 joins the second beam 675 to the third beam 676, and a third curve 673 joins the third beam 676 to the fourth beam 677. In some embodiments, the concavities of consecutive curves may face opposite directions, and the concavities of alternating curves may face the same direction. As an example, the first curve 671 and the second curve 672 are consecutive curves, while the first curve 671 and the third curve 673 are alternating curves. The concavities of the first curve 671 and the third curve 673 may face a first direction, and the concavity of the second curve 672 may face an opposite second direction.

In some embodiments, the radius of curvature of the curves may differ. In some embodiments, the radius of curvature of consecutive curves may differ, while the radius of curvature of alternating curves may be the same. For example, in the illustrative embodiment of FIG. 65, the first curve 671 and the third curve 673 may have the same radius of curvature, while the second curve 672 may have a different radius of curvature than that of the first curve 671 and the third curve 673. In some embodiments, the second curve has a smaller radius of curvature than that of the first curve and that of the third curve. In other embodiments, the second curve has a larger radius of curvature than that of the first curve and that of the third curve.

It is appreciated that, in some embodiments, having a spring with curves of different radii of curvature may permit the spring shape to have longer beams within a constrained overall footprint. Without wishing to be bound by theory, it is recognized that, in some circumstances, longer beam lengths in a spring may help promote a larger overshoot past its rest position as the spring decompresses. Furthermore, without wishing to be bound by theory, it isalso recognized that, in some circumstances, curves of different radii of curvature may help to decrease strain at concentration points.

For example, if the radius of curvature of the second curve 672 were increased, but the position of the second curve remained unchanged, the length of the second beam 675 and the third beam 676 would potentially decrease. In the illustrative embodiment of FIG. 65, the second beam 675 and the third beam 676 have the same length. As also seen in FIG. 65, in some embodiments, the first end 660 of the spring may attach to the cap 662 at an off-center location to allow the first beam 674 to have a longer length. In some embodiments, the second end 661 of the spring may attach to the hub 553 at an off-center location to allow the fourth beam 677 to have a longer length.

In some embodiments, the spring beams may be oriented in a direction that is out of plane with the latch and/or the latch release. In some embodiments, the spring beams extend in a direction that is perpendicular to the width of the latch and latch release. It is appreciated that such an arrangement may help to fit long beam lengths of the spring within a constrained footprint of the latch and spring assembly.

In the illustrative embodiment shown in FIG. 61, the latch release 766 has a width W1 spanning its two arms 663. The latch 667 also includes a width W2 in the same direction, spanning its two latch arms 603. Using the coordinate system shown in FIG. 61, the widths W1 and W2 extend in a direction along the X-axis. In contrast, the spring 664 undulates generally along the Y-axis, in a direction that is perpendicular to the widths W1 and W2. As seen in FIG. 65, the beams 674, 675, 676, and 677 of the spring 664 extend generally along the Y-axis. The spring 664 includes a width W3 along the Y-axis, which may span from the first curve 671 to the second curve 672. The width W3 of the spring 664 is perpendicular to the width W1 of the latch release, and to the width W2 of the latch. Without wishing to be bound by theory, in some embodiments, orienting the spring beam extension direction in a direction generally perpendicular to the latch and latch release widths may permit the use of longer spring beam lengths that are not physically constrained by the latch width or latch release width.

However, it should be appreciated that the specific orientation of the spring relative to the latch and latch release shown in the illustrative embodiment of FIG. 61 is not a requirement for all embodiments. In other embodiments, the spring is oriented such that the spring beam extension direction is rotated 90 degrees relative to the illustrative embodiment of FIG. 61, such that the spring undulates generally along the X-axis. In still other embodiments, the spring may be rotated any other suitable number of degrees relative to the illustrative embodiment of FIG. 61.

The spring 664 may be manufactured via injection molding, stamping, die casting, die cutting, or via any other suitable manufacturing process. The spring 664 may be made out of any suitable material, such as, but not limited to, plastic or metal.

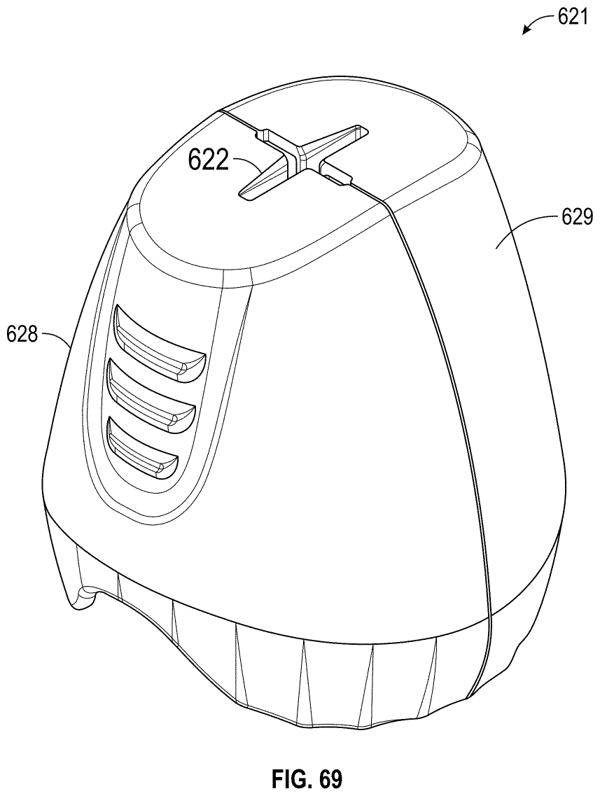

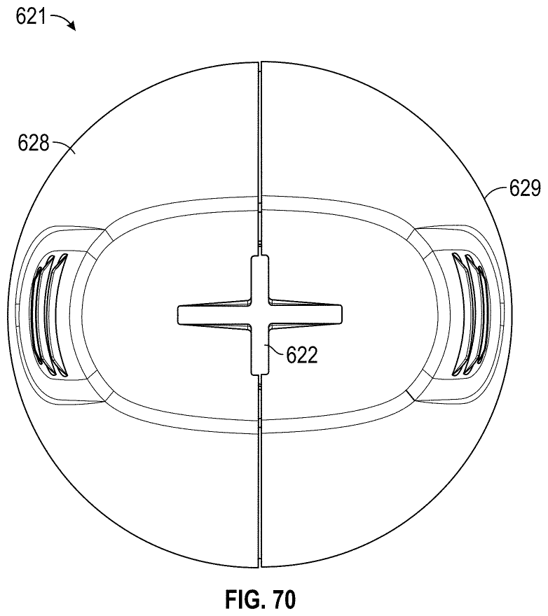

In some embodiments, device 600 may have a two-part housing 621. As shown in FIG. 60, the housing may be formed of a first housing portion 628 and a second housing portion 629. The first and second housing portions 628, 629 may be identical components that are shaped to mate with one another to form a complete housing. FIG. 69 shows the assembled housing 621. Mating of the first housing portion 628 with the second housing portion 629 may form an opening 622 through which the stem 694 of the device actuator 690 can move.

As seen in FIG. 60, in some embodiments, the storage container 650, which may be in the form of a collection tube that may have an associated cap 651, may be configured to removably couple to the device by fitting over an extension 654 of the interface 630. The storage container 650 may remain coupled with and form a seal against the extension 654 via, for example, an interference fit. The extension 654 may lead to an outlet 653 via which fluid flows into the storage container 650. In some embodiments, the extension 654 is flexible to, for example, promote ease of attachment of the storage container 650 to the extension 654.

It should be appreciated that, in some embodiments, the latch and spring assembly 665 and guide housing 680 of FIGS. 60-67 may be replaced with different latch and spring assemblies and/or guide housings. While the illustrative embodiment shown in FIGS. 60-67 utilizes an integrated, one-piece latch and spring assembly 665, it should be appreciated that the latch and spring assembly may comprise separate spring, latch, and latch release components. The spring may, in some embodiments, comprise a coil spring.

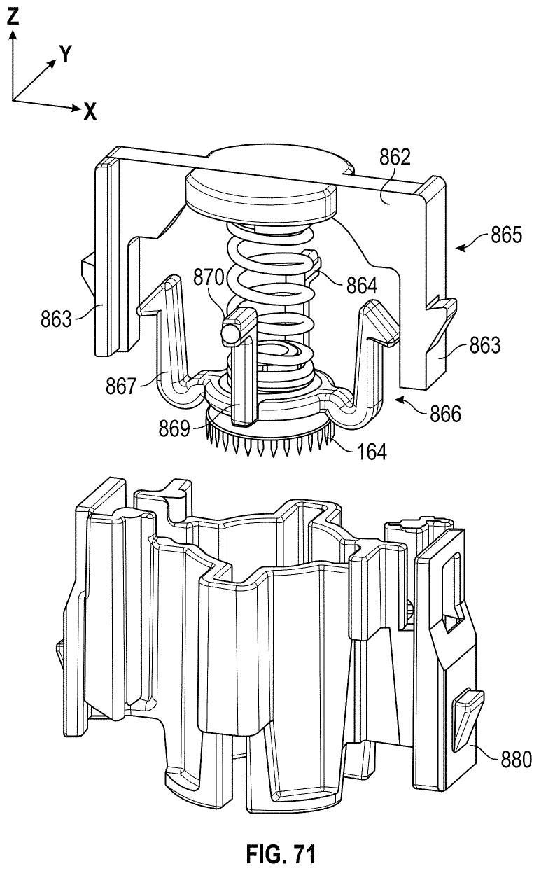

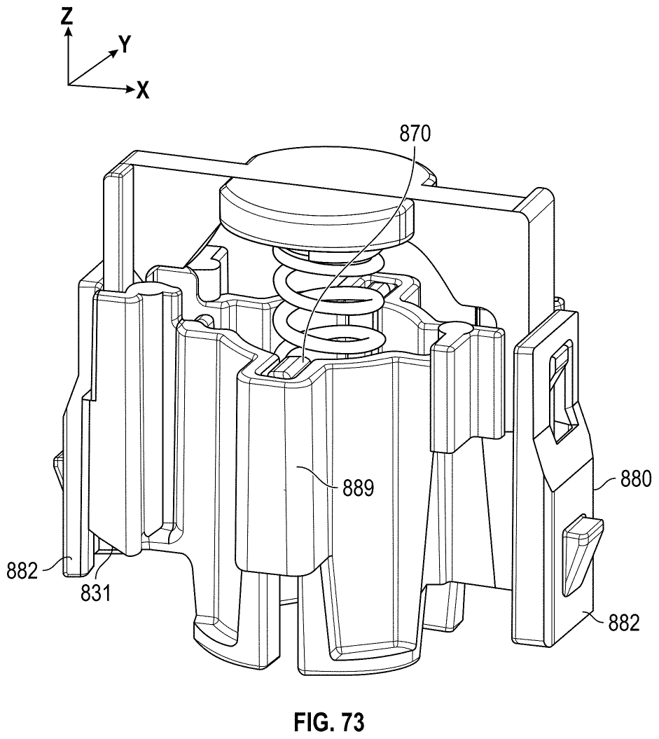

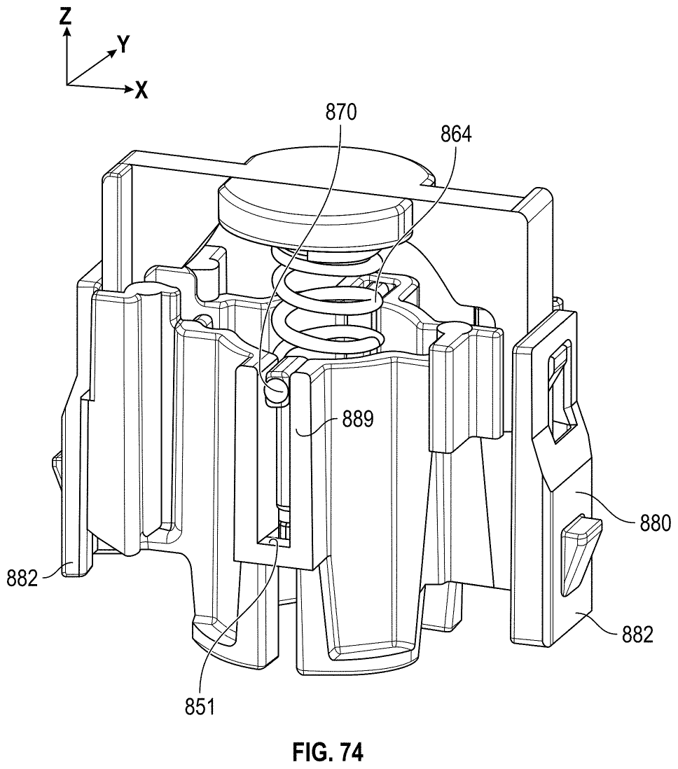

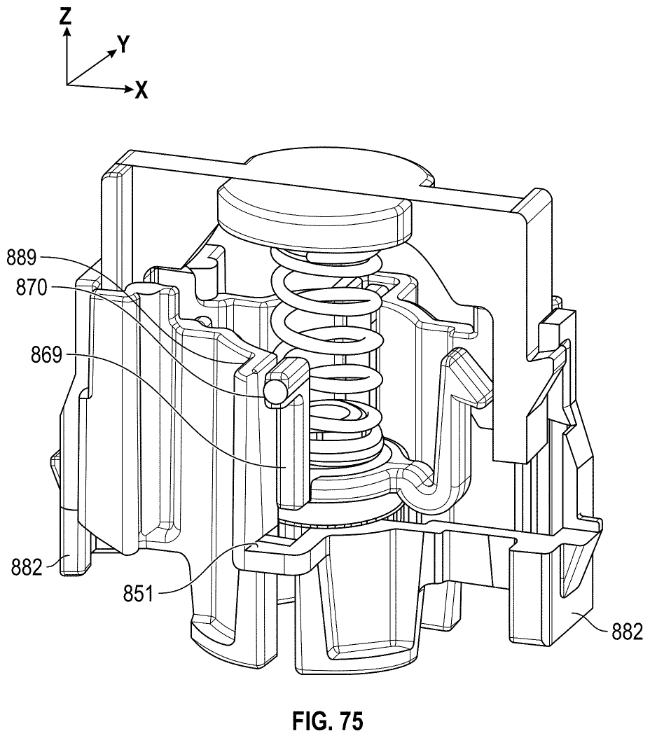

One illustrative example of an alternative embodiment of a latch and spring assembly, as well as an associated guide housing, that may be substituted into the device 600 is shown in FIGS. 71-77. The latch and spring assembly 865 includes a coil spring 864, a latch release 862, and a latch 866, where each component may be formed separately, and then attached together. The latch 866 may include a post 860 that may fit within some of the coils of the spring 864 to attach the spring to the latch. The latch 866 may have guide arms 869 that move within tracks 889 of the guide housing 880, as shown in FIGS. 73-75.

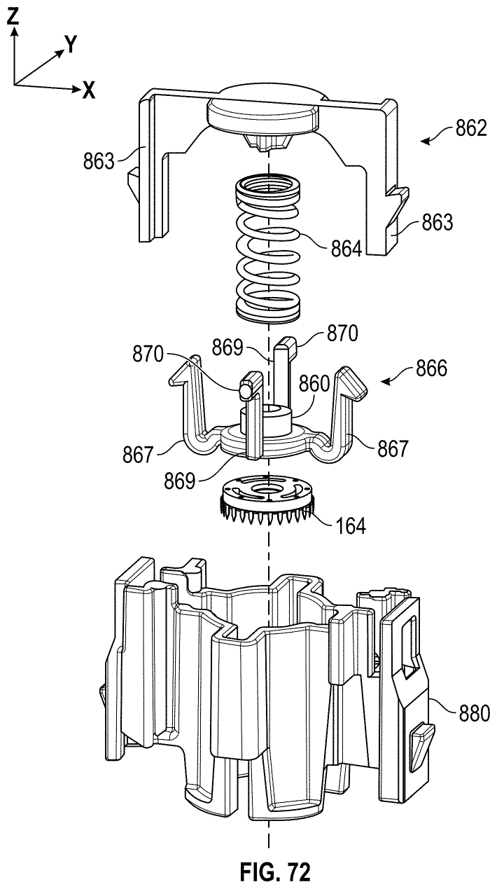

The illustrative embodiment of FIGS. 72-75 may also include a positive stop configured to limit needle movement. The positive stop may be created by interaction between the latch and the guide housing. The latch 866 may include a protruding peg 870 that may abut against a contact surface 851 of the guide housing.

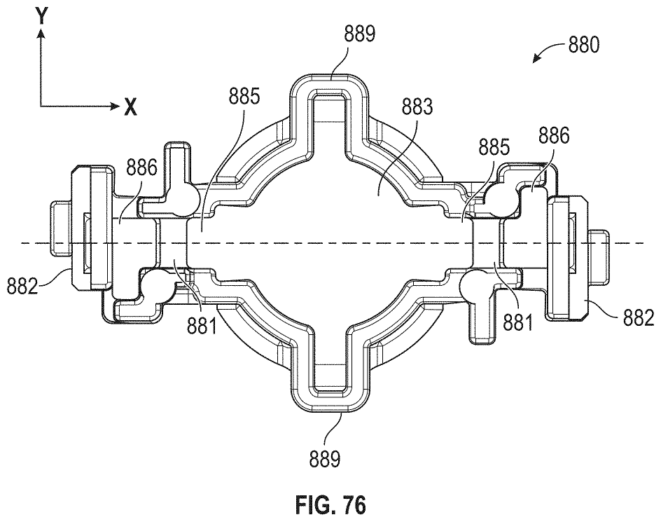

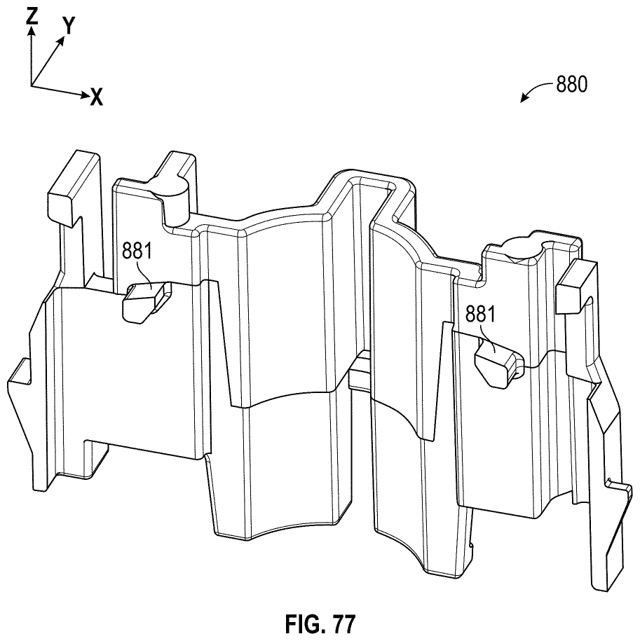

Similar to earlier embodiments, as shown in the top view of FIG. 76, the guide housing 880 may also include tracks 886 that receive and guide linear movement of the arms 863 of the latch release 866. The guide housing 880 may also include latch tracks 885 that receive and guide linear movement of the arms 867 of the latch 866. Also similar to earlier embodiments, as shown in FIG. 76 and the partial cutaway view of FIG. 77, guide housing 680 may include ledges 881 that the arms of the latch 866 initially engage with prior to device actuation. Also similar to earlier embodiments, the guide housing 880 may include wings 882 that are received by tracks on the support 200 to help constrain the guide housing from rotating and/or tilting during movement, and the guide housing may include a notch 831.

In some embodiments, the device may include an adhesive layer attached to the device interface that is configured to affix the interface to the surface of the skin. The adhesive layer may help to form a seal between the device interface and the skin, which may promote transfer of fluid from the body into the device (and/or transfer of substances from the device into the body).

As discussed above, in some embodiments, a device interface is made of a flexible material. It is appreciated the technical challenges associated with attaching an adhesive layer onto a flexible material or other low surface energy material.

In one illustrative embodiment, an adhesive layer is heat-staked onto the device interface. In some embodiments, an adhesive layer that is heat-staked to the device interface may form a seal between the adhesive layer and the device interface. In some embodiments, the heat-staked adhesive layer may be able to withstand certain sterilization processes, such as gamma sterilization.

In some embodiments, the adhesive layer is a single-sided adhesive that includes an adhesive side and a non-adhesive backer side. In some embodiments, the process of heat-staking the adhesive layer to the device interface melts the backer to the device interface, thereby attaching the adhesive layer to the device interface.

In some embodiments, including some embodiments in which the adhesive is heat-staked to the device, the material of the backer is plastic. However, the backer is not necessarily limited to plastic in all embodiments. The backer may be made of any suitable material, including wovens and non-wovens, plastic, polyphenylene ether, elastomer, elastic polymer blend nonwoven, fiber-reinforced adhesive transfer tape, knit polyester tricot fabric, polyethylene, low density polyethylene, nylon, polyvinyl chloride foam, polyester, polyester spunlace, polyethylene/ethylene vinyl acetate, polyolefin, polyolefin foam, polyolefin foam covered wires, polypropylene, urethane, polyurethane, rayon nonwoven, rayon woven fabric, spunlace nonwoven, or thermoplastic elastomer film.

In some embodiments, including some embodiments in which adhesive is heat-staked to the device, the skin-side adhesive is made of an acrylate material. However, the adhesive is not necessarily limited to acrylate in all embodiments. The skin-side adhesive may be made of any suitable material, including acrylate, hydrocolloid, acrylic-based adhesives, silicone-based adhesives, hydrogel, pressure-sensitive adhesives, a contact adhesive, or the like. In some cases, the adhesive is chosen to be biocompatible or hypoallergenic.

In some embodiments, the entire surface area of the interface bottom may be covered with an adhesive layer. In other embodiments, only a portion of the surface area of the interface bottom is covered with an adhesive layer.

In some embodiments, the adhesive-side of the adhesive layer may be initially covered with a liner that a user peels off to expose the adhesive prior to use of the device.

In another set of embodiments, the device may be mechanically held to the skin, for example, the device may include mechanical elements such as straps, belts, buckles, strings, ties, elastic bands, or the like. For example, a strap may be worn around the device to hold the device in place against the skin of the subject. In yet another set of embodiments, a combination of these and/or other techniques may be used. As one non-limiting example, the device may be affixed to a subject's arm or leg using adhesive and a strap.

In the illustrative embodiment of FIGS. 57-60, an adhesive layer 670 is attached to a bottom 631 of an interface 630 that may be flexible. The adhesive layer 670 may be heat-staked to the interface bottom 631, or attached via any other suitable arrangement, such as bonded or mechanically coupled.

In illustrative embodiments discussed above, the needle deployment and retraction mechanisms are used in devices in which vacuum is applied to the skin after needle insertion in to the skin. However, it should be appreciated that the needle deployment and retraction mechanisms described above may also be used in devices in which vacuum is applied to the skin before needle insertion into the skin. For example, if a flexible dome is used as a vacuum source, the piercing assembly could include an arrangement in which the one or more needles are triggered to deploy after the flexible dome has returned back from a compressed state to its original uncompressed state, or returned back to a near-uncompressed state. As another example, a pre-evacuated volume of space may be used as the vacuum source, in which case the device is configured to release vacuum from the pre-evacuated space prior to deployment of the needles.

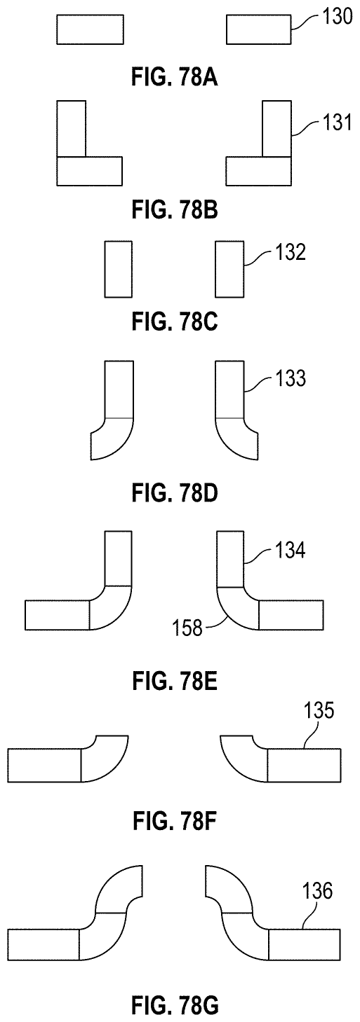

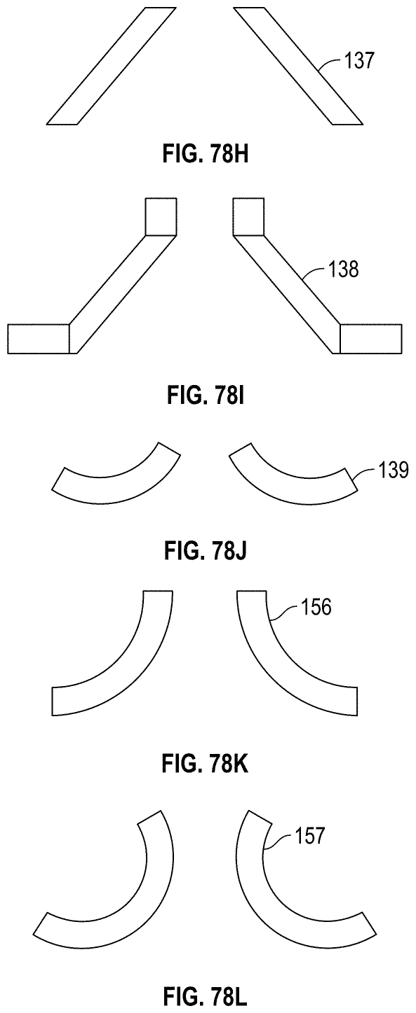

As discussed above, the shape of the support may vary between different embodiments. Various illustrative embodiments of cross-sections of support shapes are shown in FIGS. 78A-78M. It should be understood that these figures may show different shapes for only a distal portion of a support—the remaining portion of the support may be any suitable shape or have any suitable feature(s). In other embodiments, the shapes shown in FIGS. 78A-78M are the complete shape of the support, rather than just a distal portion. It should be appreciated that any of the shapes shown in FIGS. 78A-78M may be used for any of the supports in the embodiments described above, including, but not limited to, the illustrative embodiments shown in the figures.

In FIG. 78A, the support 130 has a straight-wall cross-section with an aspect ratio that is greater than 1.

In FIG. 78B, the support 131 has an L-shaped cross-section.

In FIG. 78C, the support 132 has a straight-wall cross-section with an aspect ratio that is less than 1.

In FIG. 78D, the support 133 has a J-shaped cross-section.

In FIG. 78E, the support 134 has an L-shaped cross-section with a rounded corner 158.

In FIG. 78F, the support 135 has a cross-section having a horizontal section and a rounded segment transitioning to a short vertical section.

In FIG. 78G, the support 136 has an S-shaped cross-section.

In FIG. 78H, the support 137 is funnel-shaped with straight walls.

In FIG. 78I, the support 138 is funnel-shaped with a proximal vertical section and distal a horizontal section.

In FIG. 78J, the support 139 is funnel-shaped with curved walls that have a shallow arc sweep of less than a quarter-circle, (in some embodiments, approximately one-eighth of a circle).

In FIG. 78K, the support 156 is funnel-shaped with curved walls that have an arc sweep of approximately a quarter-circle.

In FIG. 78L, the support 157 is funnel-shaped with curved, C-shaped walls.

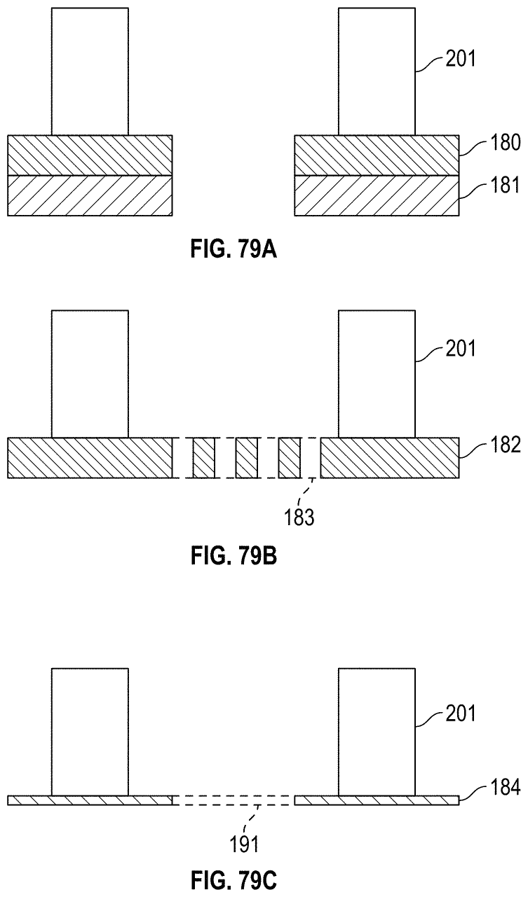

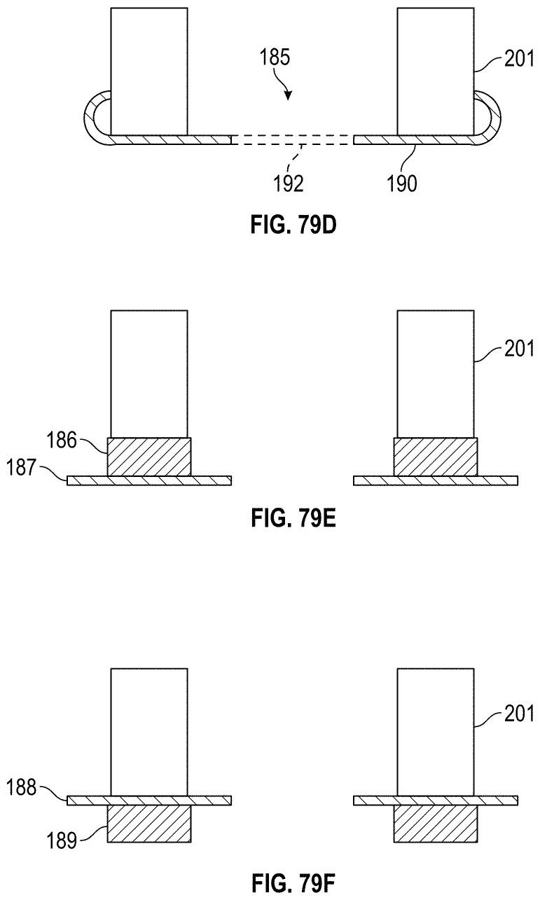

As discussed above, the arrangement of the interface may vary between different embodiments. Various illustrative embodiments of interface arrangements are shown in FIGS. 79A-79F. It should be appreciated that any of the arrangements shown in FIGS. 79A-79F may be used in any of the embodiments described above, including, but not limited to, the illustrative embodiments shown in the figures.

In FIG. 79A, the interface comprises two layers 180, 181 of different materials.

In FIG. 79B, the interface comprises a continuous layer of material having one or more channel openings 183 that permit entry of substance(s) from the body into the device.

In FIG. 79C, the interface comprises a membrane 184 with one or more channel openings 191 that permit entry of substance(s) from the body into the device.

In FIG. 79D, the interface comprises a membrane 190 attached to the side of the support 201 with slack, where the slack may permit the membrane to slide into the device opening 185 as skin moves into the device opening (e.g. due to vacuum). The membrane may have a plurality of channel openings 192.

In FIG. 79E, the interface comprises a membrane 187 with a ring of cushion material 186, the cushion material 186 being interposed between the support 201 and the membrane 187.

In FIG. 79F, the interface comprises a membrane 188 with a ring of cushion material 189, the membrane 188 being interposed between the support 201 and the cushion material 189.

In some embodiments, a deformable structure may serve as a needle deployment and/or retraction mechanism. In one set of embodiments, the deformable structure may move from a first position to a second position, and optionally the deformable structure may be able to reversibly move from the second position to the first position, i.e., the deformable structure may be a reversibly deformable structure. In some cases, the first position is stable and the second position is unstable, although in other cases both the first position and the second position are each stable (i.e., the reversibly deformable structure is bi-stable). In such a stable position, no external forces are needed to maintain equilibrium, i.e., its position.

For example, the first position may be one where the deformable structure is positioned such that the needle(s) do not contact the skin, while the second position may be one where the needle(s) contact the skin, and in some cases, the needle(s) may pierce the skin. The deformable structure may be moved using any suitable technique, e.g., manually, mechanically, electromagnetically, using a servo mechanism, or the like. In one set of embodiments, for example, the deformable structure may be moved from a first position to a second position by pushing a button, which causes the deformable structure to move (either directly, or through a mechanism linking the button with the support structure). Other mechanisms (e.g., dials, levers, sliders, etc., as discussed herein) may be used in conjunction of or instead of a button. In another set of embodiments, the deformable structure may be moved from a first position to a second position automatically, for example, upon activation by a computer, upon remote activation, after a period of time has elapsed, or the like. For example, in one embodiment, a servo connected to the deformable structure is activated electronically, moving the deformable structure from the first position to the second position.

In some cases, the deformable structure may also be moved from the second position to the first position. For example, after fluid has been delivered to and/or withdrawn from the skin and/or beneath the skin, e.g., using needle(s), the deformable structure may be moved, which may move the needle(s) away from contact with the skin. The deformable structure may be moved from the second position to the first position using any suitable technique, including those described above, and the technique for moving the support structure from the second position to the first position may be the same or different as that moving the support structure from the first position to the second position. In some cases, the deformable structure is reversibly deformable, i.e., the deformable structure is able to return from the second position back to the first position.

In one set of embodiments, the device includes a deformable structure able to drive needle(s) into the skin, e.g., so that the needle(s) can withdraw a fluid from the skin and/or from beneath the skin of a subject, and/or so that the needle(s) can deliver fluid or other material to a subject, e.g. deliver the fluid or other material to the skin and/or to a location beneath the skin of a subject. The deformable structure may be a structure that can be deformed using unaided force (e.g., by a human pushing the structure), or other forces (e.g., electrically-applied forces, mechanical interactions or the like), but is able to restore its original shape after the force is removed or at least partially reduced. For example, the structure may restore its original shape spontaneously, or some action (e.g., heating) may be needed to restore the structure to its original shape.

The deformable structure may be formed out of a suitable elastic material, in some cases. For example, the structure may be formed from a plastic, a polymer, a metal, etc. In one set of embodiments, the structure may have a concave or convex shape. For instance, the edges of the structure may be put under compressive stress such that the structure “bows” out to form a concave or convex shape. A person pushing against the concave or convex shape may deform the structure, but after the person stops pushing on the structure, the structure may be able to return to its original concave or convex shape, e.g., spontaneously or with the aid of other forces as previously discussed. In some cases, the device may be bi-stable, i.e., having two different positions in which the device is stable.

In one set of embodiments, the device may include a deformable structure that is moveable between a first configuration and a second configuration. For instance, the first configuration may have a concave shape, such as a dome shape, and the second configuration may have a different shape, for example, a deformed shape (e.g., a “squashed dome”), a convex shape, an inverted concave shape, or the like. The deformable structure may be moved between the first configuration and the second configuration manually, e.g., by pushing on the flexible concave member using a hand or a finger, and/or the deformable structure may be moved using an actuator such as is described herein. In some cases, the deformable structure may be able to spontaneously return from the second configuration back to the first configuration. In other cases, however, the deformable structure may not be able to return to the first configuration, for instance, in order to prevent accidental repeated uses of the deformable structure. The deformable structure, in some embodiments, may be a reversibly deformable structure, although in other embodiments, it need not be. In addition, in some cases, although the deformable structure may (or may not) be a reversibly deformable structure, the deformable structure may be moved from a first position to a second position using a first mechanism, and moved from the second position to the first position, or to a third position, using a second mechanism different from the first mechanism.

The deformable structure may be mechanically coupled to one or more needles (e.g., microneedles). The needle may be directly immobilized on the deformable structure, or the needles can be mechanically coupled to the deformable structure using bars, rods, levers, plates, springs, or other suitable structures. The needle(s), in some embodiments, are mechanically coupled to the deformable structure such that the needle is in a first position when the deformable structure is in a first configuration and the needle is in a second position when the deformable structure is in a second configuration.

In some cases, relatively high speeds and/or accelerations may be achieved, and/or insertion of the needle may occur in a relatively short period of time, e.g., as is discussed herein. The first position and the second position, in some cases, may be separated by relatively small distances. For example, the first position and the second position may be separated by a distance of less than about 10 mm, less than about 9 mm, less than about 8 mm, less than about 7 mm, less than about 6 mm, less than about 5 mm, less than about 4 mm, less than about 3 mm, or less than about 2 mm, etc. However, even within such distances, in certain embodiments, high speeds and/or accelerations such as those discussed herein can be achieved.

During use, a device may be placed into contact with the skin of a subject such that a recess or other suitable applicator region is proximate or in contact with the skin. By moving the deformable structure between a first configuration and a second configuration, because of the mechanical coupling, the deformable structure is able to cause a needle to move to a second position within the recess or other applicator region and to contact or penetrate the skin of the subject.

In some embodiments, the device may also include a retraction mechanism able to move the needle away from the skin after the deformable structure reaches a second configuration. Retraction of the deformable structure may, in some embodiments, be caused by the deformable structure itself, e.g., spontaneously returning from the second configuration back to the first configuration, and/or the device may include a separate retraction mechanism, for example, a spring, an elastic member, a collapsible foam, or the like. In other cases, however, a different mechanism may be used to retract the deformable structure. For example, the deformable structure may be in a second configuration, and withdrawn from the skin, e.g., laterally, without altering the configuration of the deformable structure.

The deformable structure may be formed from any suitable material, for example, a metal such as stainless steel (e.g., 301, 301LN, 304, 304L, 304LN, 304H, 305, 312, 321, 321H, 316, 316L, 316LN, 316Ti, 317L, 409, 410, 430, 440A, 440B, 440C, 440F, 904L), carbon steel, spring steel, spring brass, phosphor bronze, beryllium copper, titanium, titanium alloy steels, chrome vanadium, nickel alloy steels (e.g., Monel 400, Monel K 500, Inconel 600, Inconel 718, Inconel x 750, etc.), a polymer (e.g., polyvinylchloride, polypropylene, polycarbonate, etc.), a composite or a laminate (e.g., comprising fiberglass, carbon fiber, bamboo, Kevlar, etc.), or the like.

The deformable structure may be of any shape and/or size. In one set of embodiments, the deformable structure is not planar, and has a portion that can be in a first position (a “cocked” or predeployed position) or a second position (a “fired” or deployed position), optionally separated by a relatively high energy configuration. In some cases, both the first position and the second position are stable (i.e., the structure is bi-stable), although conversion between the first position and the second position requires the structure to proceed through an unstable configuration.

In one embodiment, the deformable structure is a flexible concave member. The deformable structure may have, for instance, a generally domed shape (e.g., as in a snap dome), and be circular (no legs), or the deformable structure may have other shapes, e.g., oblong, triangular (3 legs), square (4 legs), pentagonal (5 legs), hexagonal (6 legs), spider-legged, star-like, clover-shaped (with any number of lobes, e.g., 2, 3, 4, 5, etc.), or the like. The deformable structure may have, in some embodiments, a hole, dimple, or button in the middle. The deformable structure may also have a serrated disc or a wave shape. In some cases, the needle(s) may be mounted on the deformable structure. In other cases, however, the needle(s) are mounted on a separate structure which is driven or actuated upon movement of the deformable structure.

As used herein, “vacuum” generally refers to an amount of pressure below atmospheric pressure, such that atmospheric pressure has a vacuum of 0 mmHg, i.e., the pressure is gauge pressure rather than absolute pressure. For example, a vacuum may have a pressure of at least about 50 mmHg, at least about 100 mmHg, at least about 150 mmHg, at least about 200 mmHg, at least about 250 mmHg, at least about 300 mmHg, at least about 350 mmHg, at least about 400 mmHg, at least about 450 mmHg, at least about 500 mmHg, at least about 550 mmHg, at least about 600 mmHg, at least about 650 mmHg, at least about 700 mmHg, or at least about 750 mmHg below atmospheric pressure, i.e., a pressure that is reduced, as compared to standard atmospheric pressure. For instance, a vacuum pressure of 100 mmHg corresponds to an absolute pressure of about 660 mmHg (i.e., 100 mmHg below 1 atm).

The vacuum may be applied to any suitable region of the skin, and the area of the skin to which the vacuum may be controlled in some cases. For instance, the average diameter of the region to which vacuum is applied may be kept to less than about 5 cm, less than about 4 cm, less than about 3 cm, less than about 2 cm, less than about 1 cm, less than about 5 mm, less than about 4 mm, less than about 3 mm, less than about 2 mm, or less than about 1 mm. In addition, such vacuums may be applied for any suitable length of time. For instance, vacuum may be applied to the skin for at least about 1 min, at least about 3 min, at least about 5 min, at least about 10 min, at least about 15 min, at least about 30 min, at least about 45 min, at least about 1 hour, at least about 2 hours, at least about 3 hours, at least about 4 hours, etc. Different amounts of vacuum may be applied to different subjects in some cases, for example, due to differences in the physical characteristics of the skin of the subjects.

In some embodiments, the flow activator may include one or more needles and/or blades. In some embodiments, the needle(s) is(are) a microneedle(s). The needles may be arranged in a variety of different ways, depending on the intended application.

For example, in some embodiments, the needle(s) may have a length of less than or equal to about 5 mm, less than or equal to about 4 mm, less than or equal to about 3 mm, less than or equal to about 2 mm, less than or equal to about 1 mm, less than or equal to about 800 micrometers, less than or equal to 600 micrometers, less than or equal to about 500 micrometers, less than or equal to about 400 micrometers, less than or equal to about 300 micrometers, less than or equal to about 200 micrometers, less than or equal to about 175 micrometers, less than or equal to about 150 micrometers, less than or equal to about 125 micrometers, less than or equal to about 100 micrometers, less than or equal to about 75 micrometers, less than or equal to about 50 micrometers, less than or equal to about 10 micrometers, etc.

In some embodiments, the needle(s) may have a largest cross-sectional dimension of less than or equal to about 5 mm, less than or equal to about 4 mm, less than or equal to about 3 mm, less than or equal to about 2 mm, less than or equal to about 1 mm, less than or equal to about 800 micrometers, less than or equal to 600 micrometers, less than or equal to 500 micrometers, less than or equal to 400 micrometers, less than or equal to about 350 micrometers, less than or equal to about 300 micrometers, less than or equal to about 200 micrometers, less than or equal to about 175 micrometers, less than or equal to about 150 micrometers, less than or equal to about 125 micrometers, less than or equal to about 100 micrometers, less than or equal to about 75 micrometers, less than or equal to about 50 micrometers, less than or equal to about 10 micrometers, etc.

In some embodiments, the largest cross-sectional dimension of the needle is the width of the needle. In some embodiments, the largest cross-sectional dimension of the needle is the thickness of the needle. In some embodiments, the largest cross-sectional dimension of the needle is the diameter of the needle. Depending upon the geometry of the needle, some or all of these terms (i.e. width, thickness, diameter) may be interchangeable.

For example, some embodiments include needles having a rectangular cross-section, and may have a thickness and a width that are distinct from one another. Some embodiments include needles having a circular cross-section, where its largest cross-sectional dimension of the needle is the diameter of the circular cross-section.

In some embodiments, the needle(s) may have a rectangular cross section having dimensions of 175 micrometers by 50 micrometers, or 350 micrometers by 50 micrometers.

In one set of embodiments, the needle(s) may have an aspect ratio of length to largest cross-sectional dimension of at least about 2:1, at least about 3:1, at least about 4:1, at least 5:1, at least about 7:1, at least about 10:1, at least about 15:1, at least about 20:1, at least about 25:1, at least about 30:1, etc.

It should be understood that references to “needle” or “microneedle” as discussed herein are by way of example and ease of presentation only, and that in other embodiments, more than one needle and/or microneedle may be present in any of the descriptions herein.

As an example, microneedles such as those disclosed in U.S. Pat. No. 6,334,856, issued Jan. 1, 2002, entitled “Microneedle Devices and Methods of Manufacture and Use Thereof,” by Allen, et al., may be used to deliver to and/or withdraw fluids (or other materials) from a subject. The microneedles may be hollow or solid, and may be formed from any suitable material, e.g., metals, ceramics, semiconductors, organics, polymers, and/or composites. Examples include, but are not limited to, medical grade stainless steel, titanium, nickel, iron, gold, tin, chromium, copper, alloys of these or other metals, silicon, silicon dioxide, and polymers, including polymers of hydroxy acids such as lactic acid and glycolic acid polylactide, polyglycolide, polylactide-co-glycolide, and copolymers with polyethylene glycol, polyanhydrides, polyorthoesters, polyurethanes, polybutyric acid, polyvaleric acid, polylactide-co-caprolactone, polycarbonate, polymethacrylic acid, polyethylenevinyl acetate, polytetrafluorethylene, polymethyl methacrylate, polyacrylic acid, or polyesters.

In some cases, more than one needle or microneedle may be used. For example, arrays of needles or microneedles may be used, and the needles or microneedles may be arranged in the array in any suitable configuration, e.g., periodic, random, etc. In some cases, the array may have 3 or more, 4 or more, 5 or more, 6 or more, 10 or more, 15 or more, 20 or more, 35 or more, 50 or more, 100 or more, or any other suitable number of needles or microneedles. Typically, a microneedle will have an average cross-sectional dimension (e.g., diameter) of less than about a micron.

In one illustrative embodiment, the flow activator includes an array of microneedles that are arranged in a 7.5 mm diameter circular pattern with 30 microneedles around the circumference. Each of the microneedles is 1 mm long and 0.350 mm wide.

Those of ordinary skill in the art can arrange needles relative to the skin or other surface for these purposes including, in one embodiment, introducing needles into the skin at an angle, relative to the skin's surface, other than 90 degrees, i.e., to introduce a needle or needles into the skin in a slanting fashion so as to limit the depth of penetration. In another embodiment, however, the needles may enter the skin or other surface at approximately 90 degrees.

In some cases, the needles (or microneedles) may be present in an array selected such that the density of needles within the array is between about 0.5 needles/mm² and about 10 needles/mm², and in some cases, the density may be between about 0.6 needles/mm² and about 5 needles/mm², between about 0.8 needles/mm² and about 3 needles/mm², between about 1 needles/mm² and about 2.5 needles/mm², or the like. In some cases, the needles may be positioned within the array such that no two needles are closer than about 1 mm, about 0.9 mm, about 0.8 mm, about 0.7 mm, about 0.6 mm, about 0.5 mm, about 0.4 mm, about 0.3 mm, about 0.2 mm, about 0.1 mm, about 0.05 mm, about 0.03 mm, about 0.01 mm, etc.

In another set of embodiments, the needles (or microneedles) may be chosen such that the area of the needles (determined by determining the area of penetration or perforation on the surface of the skin of the subject by the needles) allows for adequate flow of fluid to or from the skin and/or beneath the skin of the subject. The needles may be chosen to have smaller or larger areas (or smaller or large diameters), so long as the area of contact for the needles to the skin is sufficient to allow adequate blood flow from the skin of the subject to the device. For example, in certain embodiments, the needles may be selected to have a combined skin-penetration area of at least about 500 nm², at least about 1,000 nm², at least about 3,000 nm², at least about 10,000 nm², at least about 30,000 nm², at least about 100,000 nm², at least about 300,000 nm², at least about 1 microns², at least about 3 microns², at least about 10 microns², at least about 30 microns², at least about 100 microns², at least about 300 microns², at least about 500 microns², at least about 1,000 microns², at least about 2,000 microns², at least about 2,500 microns², at least about 3,000 microns², at least about 5,000 microns², at least about 8,000 microns², at least about 10,000 microns², at least about 35,000 microns², at least about 100,000 microns², at least about 300,000 microns², at least about 500,000 microns², at least about 800,000 microns², at least about 8,000,000 microns², etc., depending on the application.

The needles or microneedles may have any suitable length, and the length may be, in some cases, dependent on the application. For example, needles designed to only penetrate the epidermis may be shorter than needles designed to also penetrate the dermis, or to extend beneath the dermis or the skin. In certain embodiments, the needles or microneedles may have a maximum penetration into the skin of no more than about 3 mm, no more than about 2 mm, no more than about 1.75 mm, no more than about 1.5 mm, no more than about 1.25 mm, no more than about 1 mm, no more than about 900 microns, no more than about 800 microns, no more than about 750 microns, no more than about 600 microns, no more than about 500 microns, no more than about 400 microns, no more than about 300 microns, no more than about 200 microns, no more than about 175 micrometers, no more than about 150 micrometers, no more than about 125 micrometers, no more than about 100 micrometers, no more than about 75 micrometers, no more than about 50 micrometers, etc. In certain embodiments, the needles or microneedles may be selected so as to have a maximum penetration into the skin of at least about 50 micrometers, at least about 100 micrometers, at least about 300 micrometers, at least about 500 micrometers, at least about 1 mm, at least about 2 mm, at least about 3 mm, etc.

In one set of embodiments, the needles (or microneedles) may be coated. For example, the needles may be coated with a substance that is delivered when the needles are inserted into the skin. For instance, the coating may comprise heparin, an anticoagulant, an anti-inflammatory compound, an analgesic, an anti-histamine compound, etc. to assist with the flow of blood from the skin of the subject, or the coating may comprise a drug or other therapeutic agent such as those described herein. The drug or other therapeutic agent may be one used for localized delivery (e.g., of or proximate the region to which the coated needles or microneedles are applied), and/or the drug or other therapeutic agent may be one intended for systemic delivery within the subject.

In some cases, at least a portion of the fluid received by the device from the subject may be stored, and/or analyzed to determine one or more analytes, e.g., a marker for a disease state, or the like. The fluid withdrawn from the subject may be subjected to such uses. The fluid may be removed using any suitable technique, e.g., as discussed herein.

Thus, the device, in one set of embodiments, may involve determination of a condition of a subject. A variety of sensors may be used, many of which are commercially readily available. In such a case, a bodily fluid received by the device may be analyzed, for instance, as an indication of a past, present and/or future condition of the subject, or to determine conditions that are external to the subject. The condition of the subject to be determined may be one that is currently existing in the subject, and/or one that is not currently existing, but the subject is susceptible or otherwise is at an increased risk to that condition. The condition may be a medical condition, e.g., diabetes or cancer, or other physiological conditions, such as dehydration, pregnancy, illicit drug use, or the like. Additional non-limiting examples are discussed herein. Determination may occur, for instance, visually, tactilely, by odor, via instrumentation, etc.

In some cases, such fluids will contain various analytes within the body that are important for diagnostic purposes, for example, markers for various disease states, such as glucose (e.g., for diabetics); other example analytes include ions such as sodium, potassium, chloride, calcium, magnesium, and/or bicarbonate (e.g., to determine dehydration); gases such as carbon dioxide or oxygen; H⁺ (i.e., pH); metabolites such as urea, blood urea nitrogen or creatinine; hormones such as estradiol, estrone, progesterone, progestin, testosterone, androstenedione, etc. (e.g., to determine pregnancy, illicit drug use, or the like); or cholesterol. Other non-limiting examples include insulin or hormones.

For instance, fluids withdrawn from the skin of the subject will often contain various analytes within the body that are important for diagnostic purposes, for example, markers for various disease states, such as glucose (e.g., for diabetics); other example analytes include ions such as sodium, potassium, chloride, calcium, magnesium, and/or bicarbonate (e.g., to determine dehydration); gases such as carbon dioxide or oxygen; H⁺ (i.e., pH); metabolites such as urea, blood urea nitrogen or creatinine; hormones such as estradiol, estrone, progesterone, progestin, testosterone, androstenedione, etc. (e.g., to determine pregnancy, illicit drug use, or the like); or cholesterol. Other examples include insulin, or hormone levels. Still other analytes include, but not limited to, high-density lipoprotein (“HDL”), low-density lipoprotein (“LDL”), albumin, alanine transaminase (“ALT”), aspartate transaminase (“AST”), alkaline phosphatase (“ALP”), bilirubin, lactate dehydrogenase, etc. (e.g., for liver function tests); luteinizing hormone or beta-human chorionic gonadotrophin (hCG) (e.g., for fertility tests); prothrombin (e.g., for coagulation tests); troponin, BNT or B-type natriuretic peptide, etc., (e.g., as cardiac markers); infectious disease markers for the flu, respiratory syncytial virus or RSV, etc.; or the like.

Other conditions/analytes that can be determined by the device include pH or metal ions, proteins, enzymes, antibodies, nucleic acids (e.g. DNA, RNA, etc.), drugs, sugars (e.g., glucose), hormones (e.g., estradiol, estrone, progesterone, progestin, testosterone, androstenedione, etc.), carbohydrates, or other analytes of interest. Other conditions that can be determined include pH changes, which may indicate disease, yeast infection, periodontal disease at a mucosal surface, oxygen or carbon monoxide levels which indicate lung dysfunction, and drug levels, both legal prescription levels of drugs such as coumadin and illegal such as cocaine or nicotine. Further examples of analytes include those indicative of disease, such as cancer specific markers such as CEA and PSA, viral and bacterial antigens, and autoimmune indicators such as antibodies to double stranded DNA, indicative of Lupus. Still other conditions include exposure to elevated carbon monoxide, which could be from an external source or due to sleep apnea, too much heat (important in the case of babies whose internal temperature controls are not fully self-regulating) or from fever. Still other potentially suitable analytes include various pathogens such as bacteria or viruses (for example, coronaviruses such as SARS-CoV-2), and/or markers produced by such pathogens. Thus, in certain embodiments, one or more analytes within the skin or within the body may be determined in some fashion, which may be useful in determining a past, present and/or future condition of the subject.

In some cases, fluids or other materials received from the subject may be used to determine conditions that are external to the subject. For example, the fluids or other materials may contain reaction entities able to recognize pathogens or other environmental conditions surrounding the subject, for example, an antibody able to recognize an external pathogen (or pathogen marker). As a specific example, the pathogen may be anthrax and the antibody may be an antibody to anthrax spores. As another example, the pathogen may be a Plasmodia (some species of which causes malaria) and the antibody may be an antibody that recognizes the Plasmodia. As yet another example, the pathogen may be a virus, such as a coronavirus (e.g., SARS-CoV-2), and the antibody may be an antibody able to bind to at least a portion of the virus, such as a spike protein, an envelope protein, a membrane protein, etc.

In some embodiments, upon determination of the fluid and/or an analyte present or suspected to be present within the fluid, a microprocessor or other controller may display a suitable signal on a display. The display may also be used to display other information, in addition or instead of the above. For example, the device may include one or more displays that indicate when the device has been used or has been expired, that indicate that sampling of fluid from a subject is ongoing and/or complete, or that a problem has occurred with sampling (e.g., clogging or insufficient fluid collected), that indicate that analysis of an analyte within the collected sample is ongoing and/or complete, that an adequate amount of a fluid has been delivered to the subject (or that an inadequate amount has been delivered, and/or that fluid delivery is ongoing), that the device can be removed from the skin of the subject (e.g., upon completion of delivery and/or withdrawal of a fluid, and/or upon suitable analysis, transmission, etc.), or the like.

However, a display is not a requirement; in other embodiments, no display may be present, or other signals may be used, for example, lights, smell, sound, feel, taste, or the like. Any of a variety of signaling or display methods, associated with analyses, can be provided including signaling visually, by smell, sound, feel, taste, or the like, in one set of embodiments. Signal structures and generators include, but are not limited to, displays (visual, LED, light, etc.), speakers, chemical-releasing chambers (e.g., containing a volatile chemical), mechanical devices, heaters, coolers, or the like. In some cases, the signal structure or generator may be integral with the device (e.g., integrally connected with a support structure for application to the skin of the subject, e.g., containing a fluid transporter such as a needle or a microneedle), or the signal structure or generator may not be integrally connected with the support structure.

In some cases, the device may contain a sensor for determining a fluid and/or an analyte within the fluid. In certain embodiments, the device may contain reagents able to interact with an analyte contained or suspected to be present within the fluid from the subject, for example, a marker for a disease state. As non-limiting examples, the sensor may contain an antibody able to interact with a marker for a disease state, an enzyme such as glucose oxidase or glucose 1-dehydrogenase able to detect glucose, or the like. The analyte may be determined quantitatively or qualitatively, and/or the presence or absence of the analyte within the withdrawn fluid may be determined in some cases.

Additional non-limiting examples of sensors include, but are not limited to, pH sensors, optical sensors, ion sensors, colorimetric sensors, a sensor able to detect the concentration of a substance, or the like, e.g., as discussed herein. For instance, in one set of embodiments, the device may include an ion selective electrode. The ion selective electrode may be able to determine a specific ion and/or ions such as K⁺, H⁺, Na⁺, Ag⁺, Pb²⁺, Cd²⁺, or the like. Various ion selective electrodes can be obtained commercially. As a non-limiting example, a potassium-selective electrode may include an ion exchange resin membrane, using valinomycin, a potassium channel, as the ion carrier in the membrane to provide potassium specificity. Those of ordinary skill in the art will be aware of many suitable commercially-available sensors, and the specific sensor used may depend on the particular analyte being sensed.

The sensor may be, for example, embedded within or integrally connected to the device, or positioned remotely but with physical, electrical, and/or optical connection with the device so as to be able to sense a chamber within the device. For example, the sensor may be in fluidic communication with fluid withdrawn from a subject, directly, via a microfluidic channel, an analytical chamber, etc. The sensor may be able to sense an analyte, e.g., one that is suspected of being in a fluid withdrawn from a subject. For example, a sensor may be free of any physical connection with the device, but may be positioned so as to detect the results of interaction of electromagnetic radiation, such as infrared, ultraviolet, or visible light, which has been directed toward a portion of the device, e.g., a chamber within the device. As another example, a sensor may be positioned on or within the device, and may sense activity in a chamber by being connected optically to the chamber. Sensing communication can also be provided where the chamber is in communication with a sensor fluidly, optically or visually, thermally, pneumatically, electronically, or the like, so as to be able to sense a condition of the chamber. As one example, the sensor may be positioned downstream of a chamber, within a channel such a microfluidic channel, or the like.

The sensor may be, for example, a pH sensor, an optical sensor, an oxygen sensor, a sensor able to detect the concentration of a substance, or the like. Other examples of analytes that the sensor may be used to determine include, but are not limited to, metal ions, proteins, nucleic acids (e.g. DNA, RNA, etc.), drugs, sugars (e.g., glucose), hormones (e.g., estradiol, estrone, progesterone, progestin, testosterone, androstenedione, etc.), carbohydrates, or other analytes of interest. Non-limiting examples of sensors include dye-based detection systems, affinity-based detection systems, microfabricated gravimetric analyzers, CCD cameras, optical detectors, optical microscopy systems, electrical systems, thermocouples and thermistors, pressure sensors, etc. The sensor can include a colorimetric detection system in some cases, which may be external to the device, or microfabricated into the device in certain cases. Various non-limiting examples of sensors and sensor techniques include colorimetric detection, pressure or temperature measurements, spectroscopy such as infrared, absorption, fluorescence, UV/visible, FTIR (“Fourier Transform Infrared Spectroscopy”), or Raman; piezoelectric measurements; immunoassays; electrical measurements, electrochemical measurements (e.g., ion-specific electrodes); magnetic measurements, optical measurements such as optical density measurements; circular dichroism; light scattering measurements such as quasielectric light scattering; polarimetry; refractometry; chemical indicators such as dyes; or turbidity measurements, including nephelometry.

In one set of embodiments, a sensor in the device may be used to determine a condition of blood present within the device. For example, the sensor may indicate the condition of analytes commonly found within the blood, for example, O₂, K⁺, hemoglobin, Na⁺, glucose, or the like. As a specific non-limiting example, in some embodiments, the sensor may determine the degree of hemolysis within blood contained within the device. Without wishing to be bound by any theory, it is believed that in some cases, hemolysis of red blood cells may cause the release of potassium ions and/or free hemoglobin into the blood. By determining the levels of potassium ions, and/or hemoglobin (e.g., by subjecting the device and/or the blood to separate cells from plasma, then determining hemoglobin in the plasma using a suitable colorimetric assay), the amount of blood lysis or “stress” experienced by the blood contained within the device may be determined. Accordingly, in one set of embodiments, the device may indicate the usability of blood (or other fluid) contained within the device, e.g., by indicating the degree of stress or the amount of blood lysis. Other examples of devices suitable for indicating the usability of blood (or other fluid) contained within the device are also discussed herein (e.g., by indicating the amount of time blood has been contained in the device, the temperature history of the device, etc.).

In some embodiments, an analyte may be determined as an “on/off” or “normal/abnormal” situation. Detection of the analyte, for example, may be indicative that insulin is needed; a trip to the doctor to check cholesterol; ovulation is occurring; kidney dialysis is needed; drug levels are present (e.g., especially in the case of illegal drugs) or too high/too low (e.g., important in care of geriatrics in particular in nursing homes). As another embodiment, however, an analyte may be determined quantitatively.

In one set of embodiments, the sensor may be a test strip, for example, test strips that can be obtained commercially. Examples of test strips include, but are not limited to, glucose test strips, urine test strips, pregnancy test strips, or the like. A test strip will typically include a band, piece, or strip of paper or other material and contain one or more regions able to determine an analyte, e.g., via binding of the analyte to a diagnostic agent or a reaction entity able to interact with and/or associate with the analyte. For example, the test strip may include various enzymes or antibodies, glucose oxidase and/or ferricyanide, or the like. The test strip may be able to determine, for example, glucose, cholesterol, creatinine, ketones, blood, protein, nitrite, pH, urobilinogen, bilirubin, leucocytes, luteinizing hormone, etc., depending on the type of test strip. The test strip may be used in any number of different ways. In some cases, a test strip may be obtained commercially and inserted into the device, e.g., before or after withdrawing blood or other fluids from a subject. At least a portion of the blood or other fluid may be exposed to the test strip to determine an analyte, e.g., in embodiments where the device uses the test strip as a sensor so that the device itself determines the analyte. In some cases, the device may be sold with a test strip pre-loaded, or a user may need to insert a test strip in a device (and optionally, withdraw and replace the test strip between uses). In certain cases, the test strip may form an integral part of the device that is not removable by a user. In some embodiments, after exposure to the blood or other fluid withdrawn from the subject, the test strip may be removed from the device and determined externally, e.g., using other apparatuses able to determine the test strip, for example, commercially-available test strip readers.

Other components may be present within the device, in some embodiments. For example, the device may contain a cover, displays, ports, transmitters, sensors, microfluidic channels, chambers, fluid channels, and/or various electronics, e.g., to control or monitor fluid transport into or out of the device, to determine an analyte present within a fluid delivered and/or withdrawn from the skin, to determine the status of the device, to report or transmit information regarding the device and/or analytes, or the like.

In some aspects, the device may include channels such as microfluidic channels, which may be used to move fluids within the device. In some cases, the microfluidic channels are in fluid communication with a needle that is used to deliver and/or withdraw fluids to or from the skin. For example, in one set of embodiments, the device may also include one or more microfluidic channels to contain fluid for delivery to the needle, e.g., from a source of fluid, and/or to withdraw fluid from the skin, e.g., for delivery to an analytical chamber within the device, to a reservoir for later analysis, or the like.

In some cases, more than one chamber may be present within the device, and in some cases, some or all of the chambers may be in fluidic communication, e.g., via channels such as microfluidic channels. In various embodiments, a variety of chambers and/or channels may be present within the device, depending on the application. For example, the device may contain chambers for sensing an analyte, chambers for holding reagents, chambers for controlling temperature, chambers for controlling pH or other conditions, chambers for creating or buffering pressure or vacuum, chambers for controlling or dampening fluid flow, mixing chambers, storage chambers for containing a fluid (e.g., withdrawn using a needle), drug chambers, or the like.

For instance, in some cases, the device may contain one or more chambers for holding or containing a fluid. In some cases, the chambers may be in fluidic communication with one or more fluid transporters and/or one or more microfluidic channels. For instance, the device may contain a chamber for containing fluid withdrawn from a subject (e.g., for storage and/or later analysis), a chamber for containing a fluid for delivery to the subject (e.g., blood, saline, optionally containing drugs, hormones, vitamins, pharmaceutical agents, or the like), etc.

In some cases, a storage chamber may contain a reagent or a reaction entity able to react with an analyte suspected of being present in the blood (or other fluid) entering the device, and in some cases, the reaction entity may be determined to determine the analyte. In some cases, the determination may be made externally of the device, e.g., by determining a color change or a change in fluorescence, etc. The determination may be made by a person, or by an external apparatus able to analyze at least a portion of the device. In some cases, the determination may be made without removing blood from the device, e.g., from the storage chamber. (In other cases, however, blood or other fluid may first be removed from the device before being analyzed.) For example, the device may include one or more sensors (e.g., ion sensors such as K+ sensors, colorimetric sensors, fluorescence sensors, etc.), and/or contain “windows” that allow light to penetrate the device. The windows may be formed of glass, plastic, etc., and may be selected to be at least partially transparent to one or a range of suitable wavelengths, depending on the analyte or condition to be determined. As a specific example, the entire device (or a portion thereof) may be mounted in an external apparatus, and light from the external apparatus may pass through or otherwise interact with at least a portion of the device (e.g., be reflected or refracted via the device) to determine the analyte and/or the reaction entity.

In some cases, the device may be designed such that portions of the device are separable. For example, a first portion of the device may be removed from the surface of the skin, leaving other portions of the device behind on the skin. In one embodiment, a stop may also be included to prevent or control the depth to which the needles or microneedles (or other fluid transporter components) are inserted into the skin, e.g., to control penetration to the epidermis, dermis, etc. As another example, a device may be modular, or include a portion that is removable from the device. For instance, blood or other bodily fluid may be received by the device in a portion (e.g., containing a storage chamber) that can be removed from the device. For instance, the removed portion can be stored, shipped to another location for analysis, or the like.

In one set of embodiments, the device contains a vacuum chamber that is also used as a storage chamber to receive blood or other fluid withdrawn from the skin of the subject into the device. For instance, blood withdrawn from a subject through or via the fluid transporter may enter the vacuum chamber due to its negative pressure (i.e., because the chamber has an internal pressure less than atmospheric pressure), and optionally stored in the vacuum chamber for later use. The fluid collected by the device can then be analyzed within the device or removed from the device for analysis, storage, etc.

In another set of embodiments, however, the device may include separate vacuum chambers and storage chambers (e.g., chambers to store fluid such as blood from the skin of the subject). The vacuum chamber and storage chambers may be in fluid communication, and may have any suitable arrangement. In some embodiments, the vacuum from the vacuum chamber may be used, at least in part, to withdraw fluid from the skin, which is then directed into a storage chamber, e.g., for later analysis or use, for example, as discussed below. As an example, blood may be withdrawn into the device, flowing towards a vacuum chamber, but the fluid may be prevented from entering the vacuum chamber. For instance, in certain embodiments, a material permeable to gas but not to a liquid such as blood may be used. For example, the material may be a membrane such as a hydrophilic or hydrophobic membrane having a suitable porosity, a porous structure, a porous ceramic frit, a dissolvable interface (e.g., formed from a salt or a polymer, etc.), or the like.

In some cases, the devices described herein can be single-stage or multi-stage. That is, the device can define a single unit that includes one or more components integrally connected to each other which cannot readily be removed from each other by a user, or can include one or more components which are designed to be and can readily be removed from each other. As a non-limiting example of the later, a two-stage device can be provided for application to the skin of a subject. The device can include a first portion designed to reside proximate the skin of the subject for the duration of the analysis, which might include an analysis region, a reservoir or other material for creating vacuum or otherwise promoting the flow of fluid or other materials relative to the analysis region, a needle or a microneedle to access interstitial fluid or blood, or the like. A second stage or portion of the device can be provided that can initiate operation of the device.

For example, the two-stage device can be applied to the skin of the user. A button or other component or switch associated with the second portion of the device can be activated by the subject to cause insertion of a needle or a microneedle to the skin of the subject, or the like. Then, the second stage can be removed, e.g., by the subject, and the first stage can remain on the skin to facilitate analysis.

In another example, a two-stage device can be provided where the first stage or portion includes visualization or other signal-producing components and the second stage or portion includes components necessary to facilitate the analysis, e.g., the second stage or portion can include all components necessary to access bodily fluid, transport the fluid (if necessary) to a site of analysis, and the like, and that stage can be removed, leaving only a visualization stage for the subject or another entity to view or otherwise analyze as described herein.

In yet another example, a two-stage device can include a first stage or portion that is applied to the skin of the subject, and a second stage or portion that stores blood or another bodily fluid. The second stage can be removed and stored, shipped to another location for analysis, or the like.

In certain embodiments, portions of the device may be constructed and arranged to be connectable and/or detachable from each other readily, e.g., by the subject. Thus, for instance, the subject (or another person) may be able to connect the portions (e.g., modules) to assemble a device, and/or disconnect the portions, without the use of tools such as screwdrivers or tape. In some cases, the connection and/or disconnection can occur while the device is affixed to the skin. Thus, for example, a device may be applied to the subject of the skin, and after use, a portion of the device may be removed from the skin of the subject, leaving the remainder of the device in place on the skin. Optionally, the portion may be replaced by another portion of the device, which may be the same or different than the removed portion.

As an example, in one embodiment, a device may be fabricated to contain a first module, and a second module that is constructed and arranged for repeated connection and disconnection to the first module. The first module may, for example, be used to deliver to and/or withdraw fluid from a subject. For instance, as discussed herein, the first module may contain a fluid transporter for delivering to and/or withdrawing fluid from the skin and/or beneath the skin of the subject. The fluid may optionally be analyzed within the first module, and/or stored for later use, e.g., in a collection chamber. After withdrawal of sufficient fluid, the first module may be removed, leaving the second module in place, and optionally replaced with a new first module for subsequent use (e.g., for subsequent delivery and/or withdrawal of fluid at a later time). In other embodiments, however, the second module may be removed, leaving the first module in place. Depending on the application, the removed module may be reused or disposed of (e.g., thrown in the trash), or the module may be shipped to another location for disposal and/or analysis, for example, to analyze fluid contained within the module, e.g., withdrawn from the skin of the subject. A module may be used once, or multiple times, before being removed from the device, depending on the application. Thus, as non-limiting examples the device may contain removable modules containing removable fluid transporters (e.g., needles or microneedles), removable modules for containing blood or another fluid, e.g., which can be shipped to another location, or the like.

In some aspects, any of the following components may independently be modular and may be single-use or reusable, or even absent in some cases: a pressure regulator such as a vacuum chamber or other vacuum source, an actuator, an activator, a fluid transporter, a fluid assay, a sensor, a fluid storage (e.g., a collection chamber), a data storage or memory component, a processor, a detector, a power source, a transmitter, a display, or the like. As non-limiting examples, one module could be a single use module (e.g., modules containing one or more of the following: fluid transporter, actuator, vacuum source, fluid processing, fluid storage, assay chemistry, etc.), or a module could be a re-usable module (e.g., modules containing one or more of the following: detector, processor, data storage, display, transmitter, power source, etc.) could be a re-useable module. Alternatively, just a single unit (e.g., a fluid transporter, e.g., one or more needles or microneedles) might be single use, and the rest of the device might be re-useable. Other combinations of these are also contemplated. In certain embodiments, the replaceable portion within the device is one that is required for the device to function, for example the device may not be able to function to deliver and/or withdraw fluid without the replaceable portion being present within the device. In one embodiment, the replaceable portion is not a power source (e.g., a battery).

In one set of embodiments, the device, or a portion thereof (e.g., a module) is reusable. For instance, the device may be used repeatedly (at the same location on the skin of a subject, or at different locations) to deliver to and/or withdraw fluid from the skin and/or beneath the skin of the subject. The device used repeatedly may be a single, integral device, and/or the device may contain one or more modules such as those previously discussed. For example, in some cases, between uses, a module may be removed and/or replaced from the device, e.g., as discussed above.

In one set of embodiments, a device as discussed herein (or a portion thereof) may be shipped or transported to another location for analysis. For example, the device or a module may be hand-carried, mailed, etc. In some cases, the device may include an anticoagulant or a stabilizing agent contained within the device, e.g., within a storage chamber for the fluid. Thus, for example, fluid such as blood withdrawn from the skin may be delivered to a chamber (e.g., a storage chamber) within the device, then the device, or a portion of the device (e.g., a module) may be shipped to another location for analysis. Any form of shipping or transport may be used, e.g., via mail or hand-delivery.

After withdrawal of the fluid into the device, the device, or a portion thereof, may be removed from the skin of the subject, e.g., by the subject or by another person. For example, the entire device may be removed, or a portion of the device containing the storage reservoir may be removed from the device, and optionally replaced with another storage reservoir. Thus, for instance, in one embodiment, the device may contain two or more modules, for example, a first module that is able to cause withdrawal of fluid from the skin into a storage reservoir, and a second module containing the storage module. In some cases, the module containing the storage reservoir may be removed from the device.

As another example, the device may include at least two modules manually separable from each other, including a first module comprising a vacuum chamber, and a second module comprising other components such as those described herein. In some embodiments, the modules may be separable without the use of tools. For example, the second module may include one or more components such as a fluid transporter (e.g., a needle or microneedle), an applicator region such as a recess, a reversibly deformable structure such as a flexible concave member, a collection chamber, a sensor, a processor, or the like. As a specific example, the first module may be defined entirely or partially by a vacuum chamber, and the first module may be removed and replaced with a fresh vacuum chamber, during or between uses. Thus, for instance, the first module may be inserted into the device when blood or other bodily fluids are desired to be withdrawn from a subject, and optionally, used to cause blood to be withdrawn from the skin of the subject.

In one set of embodiments, the first module may be substantially cylindrical, and in some embodiments, the first module may be a Vacutainer™ tube, a Vacuette™ tube, or other commercially-available vacuum tube, or other vacuum source such as is described herein. In some embodiments, a Vacutainer™ or Vacuette™ tube that is used may have a maximum length of no more than about 75 mm or about 100 mm and a diameter of no more than about 16 mm or about 13 mm. The device, in certain embodiments, may also contain an adaptor able to hold or immobilize such tubes on the device, for example, a clamp. Other examples of adaptors are discussed in detail herein. In some cases, the device may have a shape or geometry that mimics a Vacutainer™ or Vacuette™ tube, e.g., one having the above dimensions. The device, in some embodiments, is substantially cylindrically symmetric.

The withdrawn fluid may then be sent to a clinical and/or laboratory setting, e.g., for analysis. In some embodiments, the entire device may be sent to the clinical and/or laboratory setting; in other embodiments, however, only a portion of the device (e.g., a module containing a storage reservoir containing the fluid) may be sent to the clinical and/or laboratory setting. In some cases, the fluid may be shipped using any suitable technique (e.g., by mail, by hand, etc.). In certain instances, the subject may give the fluid to appropriate personnel at a clinical visit. For instance, a doctor may prescribe a device as discussed above for use by the subject, and at the next doctor visit, the subject may give the doctor the withdrawn fluid, e.g., contained within a device or module.

One aspect is directed to an adaptor able to position a device in apparatuses designed to contain Vacutainer™ tubes or Vacuette™ tubes. In some cases, the Vacutainer™ or Vacuette™ tube sizes have a maximum length of no more than about 75 mm or about 100 mm and a diameter of no more than about 16 mm or about 13 mm. In some cases, the adaptor may be able to immobilize a device therein, e.g., for subsequent use or processing. In some cases, devices may have a largest lateral dimension of no more than about 50 mm, and/or a largest vertical dimension, extending from the skin of the subject when the device is applied to the subject, of no more than about 10 mm. The device may contained within the adaptor using any suitable technique, e.g., using clips, springs, braces, bands, or the application of force to the device present within the adaptor.

According to one aspect, the device is of a relatively small size. For example, in some embodiments, the device may have a largest lateral dimension (e.g., parallel to the skin) of no more than about 25 cm, no more than about 10 cm, no more than about 7 cm, no more than about 6 cm, no more than about 5.5 cm, no more than about 5 cm, no more than about 4.5 cm, no more than about 4 cm, no more than about 3.5 cm, no more than about 3 cm, no more than about 2 cm, or no more than about 1 cm. In some cases, the device may have a largest lateral dimension of between about 0.5 cm and about 1 cm, between about 2 and about 3 cm, between about 2.5 cm and about 5 cm, between about 2 cm and about 7 cm, etc.

In some embodiments, the device is relatively lightweight. For example, the device may have a mass of no more than about 1 kg, no more than about 300 g, no more than about 150 g, no more than about 100 g, no more than about 50 g, no more than about 30 g, no more than about 25 g, no more than about 20 g, no more than about 10 g, no more than about 5 g, or no more than about 2 g. For instance, in various embodiments, the device has a mass of between about 2 g and about 25 g, a mass of between about 2 g and about 10 g, a mass of between 10 g and about 50 g, a mass of between about 30 g and about 150 g, etc.

Combinations of these and/or other dimensions are also possible in other embodiments. As non-limiting examples, the device may have a largest lateral dimension of no more than about 5 cm, a largest vertical dimension of no more than about 1 cm, and a mass of no more than about 25 g; or the device may have a largest lateral dimension of no more than about 5 cm, a largest vertical dimension of no more than about 1 cm, and a mass of no more than about 25 g; etc. As additional non-limiting examples, the device may have dimensions of no more than 2.0 cm×3.1 cm×5.7 cm (height×width×length), no more than 2.5 cm×3.5 cm×6.0 cm, no more than about 1.5 cm×4.2 cm×4.7 cm, no more than 2.0 cm×4.5 cm×5.0 cm, no more than 100 mm×50 mm×100 mm, no more than 150 mm×100 mm×150 mm, no more than 200 mm×100 mm×200 mm, etc.

In some embodiments, the device may be sized such that it is wearable and/or able to be carried by a subject. For example, the device may be self-contained, needing no wires, cables, tubes, external structural elements, or other external support. The device may be positioned on any suitable position of the subject, for example, on the arm or leg, on the back, on the abdomen, etc.

In some embodiments, the device may be connected to an external apparatus for determining at least a portion of the device, a fluid removed from the device, an analyte suspected of being present within the fluid, or the like. For example, the device may be connected to an external analytical apparatus, and fluid removed from the device for later analysis, or the fluid may be analyzed within the device in situ, e.g., by adding one or more reaction entities to the device, for instance, to a storage chamber, or to analytical chamber within the device. For example, in one embodiment, the external apparatus may have a port or other suitable surface for mating with a port or other suitable surface on the device, and blood or other fluid can be removed from the device using any suitable technique, e.g., using vacuum or pressure, etc. The blood may be removed by the external apparatus, and optionally, stored and/or analyzed in some fashion. For example, in one set of embodiments, the device may include an exit port for removing a fluid from the device (e.g., blood). In some embodiments, fluid contained within a storage chamber in the device may be removed from the device, and stored for later use or analyzed outside of the device. In some cases, the exit port may be separate from the fluid transporter.

In one aspect, the device may be interfaced with an external apparatus able to determine an analyte contained within a fluid in the device, for example within a storage chamber as discussed herein. For example, the device may be mounted on an external holder, the device may include a port for transporting fluid out of the device, the device may include a window for interrogating a fluid contained within the device, or the like.

In some embodiments, the device may be connected to an external apparatus for determining at least a portion of the device, a fluid removed from the device, an analyte suspected of being present within the fluid, or the like. For example, the device may be connected to an external analytical apparatus, and fluid removed from the device for later analysis, or the fluid may be analyzed within the device in situ, e.g., by adding one or more reaction entities to the device, for instance, to a storage chamber, or to analytical chamber within the device. For example, in one embodiment, the external apparatus may have a port or other suitable surface for mating with a port or other suitable surface on the device, and blood or other fluid can be removed from the device using any suitable technique, e.g., using vacuum or pressure, etc. The blood may be removed by the external apparatus, and optionally, stored and/or analyzed in some fashion. For example, in one set of embodiments, the device may include an exit port for removing a fluid from the device (e.g., blood). In some embodiments, fluid contained within a storage chamber in the device may be removed from the device, and stored for later use or analyzed outside of the device. In some cases, the exit port may be separate from the fluid transporter. For example, an exit port can be in fluidic communication with a vacuum chamber, which can also serve as a fluid reservoir in some cases. Other methods for removing blood or other fluids from the device include, but are not limited to, removal using a vacuum line, a pipette, extraction through a septum instead of an exit port, or the like. In some cases, the device may also be positioned in a centrifuge and subjected to various g forces (e.g., to a centripetal force of at least 50 g), e.g., to cause at separation of cells or other substances within a fluid within the device to occur.

In some cases, the device may include a drug or a therapeutic agent for delivery to a subject. For example, the drug may include an anti-inflammatory compound, an analgesic, or an anti-histamine compound. Examples of anti-inflammatory compounds include, but are not limited to, NSAIDs (non-steroidal anti-inflammatory drugs) such as aspirin, ibuprofen, or naproxen. Examples of analgesics include, but are not limited to, benzocaine, butamben, dibucaine, lidocaine, oxybuprocaine, pramoxine, proparacaine, proxymetacaine, tetracaine, acetaminophen, NSAIDs such as acetylsalicylic acid, salicylic acid, diclofenac, ibuprofen, etc., or opioid drugs such as morphine or opium, etc. Examples of anti-histamine compounds include, but are not limited to, clemastine, diphenhydramine, doxylamine, loratadine, desloratadine, fexofenadine, pheniramine, cetirizine, ebastine, promethazine, chlorpheniramine, levocetirizine, olopatadine, quetiapine, meclizine, dimenhydrinate, embramine, dimethindene, dexchlorpheniramine, vitamin C, cimetidine, famotidine, ranitidine, nizatidine, roxatidine, or lafutidine. Other specific non-limiting examples of therapeutic agents that could be used include, but are not limited to biological agents such as erythropoietin (“EPO”), alpha-interferon, beta-interferon, gamma-interferon, insulin, morphine or other pain medications, antibodies such as monoclonal antibodies, or the like.

As mentioned, the device may include an anticoagulant or a stabilizing agent for stabilizing the fluid withdrawn from the skin. As a specific non-limiting example, an anticoagulant may be used for blood withdrawn from the skin. For example, the anticoagulant or stabilizing agent may be present within a storage chamber of the device.

Examples of anticoagulants include, but are not limited to, heparin, citrate, oxalate, or ethylenediaminetetraacetic acid (EDTA). Other agents may be used in conjunction or instead of anticoagulants, for example, stabilizing agents such as solvents, diluents, buffers, chelating agents, antioxidants, binding agents, preservatives, antimicrobials, or the like. Examples of preservatives include, for example, benzalkonium chloride, chlorobutanol, parabens, or thimerosal. Non-limiting examples of antioxidants include ascorbic acid, glutathione, lipoic acid, uric acid, carotenes, alpha-tocopherol, ubiquinol, or enzymes such as catalase, superoxide dismutase, or peroxidases. Examples of microbials include, but are not limited to, ethanol or isopropyl alcohol, azides, or the like. Examples of chelating agents include, but are not limited to, ethylene glycol tetraacetic acid or ethylenediaminetetraacetic acid. Examples of buffers include phosphate buffers such as those known to ordinary skill in the art.

The device may be used with an analgesic or other agent that alters or inhibits sensation. For example, an analgesic such as benzocaine, butamben, dibucaine, lidocaine, oxybuprocaine, pramoxine, proparacaine, proxymetacaine, or tetracaine may be applied to the skin, prior to or during delivery and/or withdrawal of fluid, or another obscuring agent may be applied, e.g., an agent to cause a burning sensation, such as capsaicin or capsaicin-like molecules, for example, dihydrocapsaicin, nordihydrocapsaicin, homodihydrocapsaicin, homocapsaicin, or nonivamide. Further examples of analgesics include, but are not limited to, acetaminophen, NSAIDs such as acetylsalicylic acid, salicylic acid, diclofenac, ibuprofen, etc., or opioid drugs such as morphine or opium, etc.

The analgesic or other agent may be applied to the skin using any suitable technique, e.g., using the device, or separately. The analgesic or other agent may be applied to the skin automatically, or upon activation of the device as discussed herein. For example, the analgesic or other agent may be delivered to the skin (e.g., via a microfluidic channel from a chamber containing the analgesic or other agent) prior to, and/or after, exposure of the skin to a fluid transporter as discussed herein. In some cases, the analgesic or other agent may be sprayed on the skin, e.g., through a nozzle. In another embodiment, a sponge, gauze, a swab, a membrane, a filter, a pad, or other absorbent material may be applied to the skin (e.g., by the device) to apply the analgesic or other agent to the skin, e.g., to blood or other bodily fluids present on the skin. In some cases, a fluid transporter may pass through the material. For example, upon application of the device to the skin, a portion of the device (e.g., a cover) may be moved, thereby exposing the skin to material contained within the device that contains the analgesic or other agent to be applied to the skin. In some cases, an applicator, such as a brush, a pad, or a sponge, may be moved on the surface of the skin to apply the analgesic or other agent the skin. For example, the device may move an applicator across the surface of the skin.

In one aspect, the device may include a system for sanitizing at least a portion of the skin of the subject, for example, the region of skin where fluid is delivered and/or withdrawn. The region may be sanitized at any suitable time. For instance, the region may be sanitized before, during, and/or after delivery to and/or withdrawal of fluid from the skin and/or beneath the skin of the subject. In some embodiments, the system sanitizing the skin may be formed as an integral part of the device; in other embodiments, however, the system may be contained within a module that is connectable and/or detachable to the remainder of the device (e.g., to other modules within the device). For example, the device may contain a sterilization module that optionally can be removed from the device and/or replaced with a new sterilization module, in various embodiments.

As used herein, “sanitizing” means that at least some of the microorganisms present on the surface of the skin are killed and/or inactivated (e.g., rendered uninfectious). The microorganisms that may be present include, for example, bacteria (e.g., of the genuses Propionibacteria, Corynebacteria, Staphylococcus, and/or Streptococcus, etc.), fungi, viruses (e.g., coronaviruses, such as SARS-CoV-2), or the like. It should be understood, however, that the skin may be “sanitized” without necessarily killing 100% of the microorganisms present on the skin in the region being sanitized. For example, the sanitization system may be effective at killing and/or inactivating at least 25%, at least 50% or at least 75% of the microorganisms, or by killing and/or inactivating the microorganisms by 1, 2, 3, or 4 logs, where a “log” is a 10-fold reduction in the number of active microorganisms.

In one set of embodiments, the device contains a fluid containing a sanitizer, and the fluid is applied to the skin. The fluid may be applied to the skin automatically, or upon activation of the device as discussed herein. For example, the fluid may be delivered to the skin (e.g., via a microfluidic channel from a chamber containing the fluid) prior to, and/or after, exposure of the skin to a fluid transporter as discussed herein. In some cases, the fluid may be sprayed on the skin, e.g., through a nozzle. In another embodiment, a sponge, gauze, a swab, a membrane, a filter, a pad, or other absorbent material may be applied to the skin (e.g., by the device) to sanitize the skin. In some cases, a fluid transporter may pass through the material. As another example, a portion of the device (e.g., a cover) may be moved, thereby exposing the skin to material contained within the device that contains the sanitizer. In some cases, an applicator, such as a pad, a brush or a sponge, may be moved on the surface of the skin to sanitize the skin. For example, the device may move an applicator across the surface of the skin.

In one set of embodiments, the sanitizer is a liquid, gel, or foam, and/or is contained in a liquid, gel, or foam. The sanitizer may be any suitable agent able to sanitize the skin, for example, a peroxide (e.g., H₂O₂), bleach, an alcohol (e.g., ethyl alcohol, isopropyl alcohol, etc.), n-propanol, triclosan, benzalkonium chloride, tincture of iodine (e.g., containing 2-7% potassium iodide or sodium iodide, and elemental iodine, dissolved in a mixture of ethanol and water), povidone-iodine (e.g., Betadine) chlorhexidine gluconate, or soap (e.g., common soap, such as liquid soap), or the like. In another set of embodiments, however, the sanitizer may take the form of a source of radiation, for example, ultraviolet radiation.

Other aspects are directed to a kit including one or more devices such as previously discussed. The kit may include a package or an assembly including one or more of the devices such as described herein, and/or other components associated with such devices, for example, as previously described. For example, in one set of embodiments, the kit may include a device and one or more compositions for use with the device. Each of the compositions of the kit, if present, may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions or components include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, tapes, adhesives, and the like, for example, for using, administering, modifying, assembling, storing, packaging, preparing, mixing, diluting, and/or preserving the compositions components for a particular use, for example, to a sample and/or a subject.

A kit may, in some cases, include instructions in any form that are provided in connection with a device in such a manner that one of ordinary skill in the art would recognize that the instructions are to be associated with the device. For instance, the instructions may include instructions for using, modifying, storing, shipping, repairing, dissembling, etc. the device. In some cases, the instructions may also include the use, modification, mixing, diluting, preserving, administering, assembly, storage, packaging, and/or preparation of the compositions and/or other compositions associated with the kit. In some cases, the instructions may also include instructions for the delivery and/or administration of the device, for example, for a particular use, e.g., to a subject. The instructions may be provided in any form recognizable as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

In some embodiments, a fluid receiving device may include features that are generally directed to separating blood into plasma or serum, and a portion enriched in blood cells, for example, under vacuum or reduced pressure. For example, a device may draw blood (or other suitable bodily fluids) into the device and/or through a membrane, such as a separation membrane. In some embodiments, the membrane is used to separate the blood into a first portion formed of plasma or serum, and a second portion that is concentrated in blood cells.

In some cases, the device may be used to separate a relatively small amount of blood into plasma or serum and a portion concentrated in blood cells. For example, less than about 10 ml, less than about 5 ml, less than about 3 ml, less than about 2 ml, less than about 1.5 ml, less than about 1 ml, less than about 800 microliters, less than about 600 microliters, less than about 500 microliters, less than about 400 microliters, less than about 300 microliters, less than about 200 microliters, less than about 100 microliters, less than about 80 microliters, less than about 60 microliters, less than about 40 microliters, less than about 20 microliters, less than about 10 microliters, or less than about 1 microliter of blood may be received into the device and separated within the device. The plasma or serum can then be recovered from the device, for example, using a needle to remove at least a portion of the plasma or serum, and subjected to various diagnostics or testing protocols, for example, for the detection of infections, diabetes (e.g., sugar), AIDS (e.g., HIV), cancer (e.g., prostate-specific antigen), or other indications. In some embodiments, the device may be relatively small, in contrast with machines (such as dialysis machines) that are typically used in plasmapheresis. For example, the device may be handheld or be applied to the skin of a subject, e.g., using an adhesive, as is discussed below. The device may be self-contained in some embodiments, i.e., such that the device is able to function to withdraw blood (or other bodily fluids) from a subject and separate it to produce plasma or serum without requiring external connections such as an external source of vacuum, an external source of power, or the like. For instance, a vacuum source within the device, e.g., a vacuum chamber, may be used to draw blood across the separation membrane to produce plasma or serum.

Furthermore, in certain embodiments, the device is able to effectively produce a relatively small amount of plasma or serum without requiring a relatively large amount of blood and/or without requiring a centrifuge to produce plasma or serum from the received blood. In some cases, at least about 30%, at least about 40%, at least about 50%, at least about 60%, at least about 70%, at least about 80%, or at least about 90% of the plasma or serum produced by the device may be received from the device, e.g., for use in subsequent testing or diagnostics. In contrast, in many prior art techniques where a sample of plasma or serum is required, e.g., for diagnostics or testing purposes, a relatively large volume of blood is received from a subject into a test tube (e.g., having a volume of at least 2 ml, at least 4 ml, at least 6 ml, or at least about 10 ml, such as in the Vacutainer™ (Becton, Dickinson and company) or Vacuette™ (Greiner Bio-One GmBH) systems), then the test tube is processed (for example, via centrifugation) to separate the blood from the plasma or serum. A portion of the plasma or serum is then removed from the test tube for diagnostics or testing purposes; however, the remainder of the plasma or serum within the test tube is not needed for subsequent testing or diagnostics, and is essentially wasted. Additionally, in some embodiments, serum may be produced without use of an anticoagulant within the device, although in other embodiments, the device may contain an anticoagulant to produce plasma. In some embodiments, the membrane and/or the storage chamber may contain an anticoagulant to produce plasma. Alternatively, if there is no anticoagulant present in the device, fluid that flows through a separation membrane into the storage chamber is free of blood cells and will ultimately clot in the storage chamber, thereby producing a liquid component, also known as serum. This serum can be collected via aspiration or other suitable method out of the storage chamber, leaving the blood clots in the storage chamber. Thus, many embodiments described herein may be used to produce plasma or serum, depending on the presence or absence of anticoagulant.

As mentioned, in one aspect, blood received from a subject into a device may be separated within the device to form plasma or serum by passing the blood, or at least a portion thereof, through a separation membrane or a membrane that is permeable to fluids but is substantially impermeable to cells. The separation membrane can be any membrane able to separate blood passing therethrough into a first portion (passing through the membrane) that is enriched in plasma or serum, and a second portion (rejected by the membrane) concentrated in blood cells. In some cases, the separation membrane may have a separation effectiveness during use (the separation effectiveness is the volume of plasma or serum that passes through the membrane relative to the starting volume of whole blood) of at least about 5%, at least about 10%, at least about 20%, at least about 40%, at least about 50%, at least about 55%, or at least about 60%.

In one set of embodiments, the separation membrane is selected to have a pore size smaller than the average or effective diameter of blood cells contained within the blood, including red blood cells and white blood cells. For instance, the pore size of the separation membrane may be less than about 30 micrometers, less than about 20 micrometers, less than about 10 micrometers, less than about 8 micrometers, less than about 6 micrometers, less than about 4 micrometers, less than about 3 micrometers, less than about 2 micrometers, less than about 1.5 micrometers, less than about 1 micrometer, less than about 0.5 micrometers, etc. As specific non-limiting examples, the pore size may be between about 0.5 micrometers and about 2 micrometers, or between about 0.5 micrometers and about 1 micrometer. In addition, in some embodiments, the separation membrane may have a thickness of less than about 1 mm, less than about 750 micrometers, less than about 500 micrometers, less than about 400 micrometers, less than about 350 micrometers, less than about 300 micrometers, less than about 250 micrometers, or less than about 200 micrometers.

The separation membrane may be formed out of any suitable material. For example, in some embodiments, the separation membrane may be formed out of a material that promotes thrombolysis or inhibits clot formation, such as a polyester, and/or the separation membrane may be formed and/or coated with a biocompatible material, or at least a material that does not cause an active clotting response within the blood that the separation membrane is exposed to. As specific non-limiting example, the separation membrane can comprise or be formed from glass (e.g., glass fibers), and/or a polymer such as a polycarbonate, a polysulfone, a polyethersulfone, a polyarylethersulfone, a polyvinylpyrrolidone, a polypropylene, poly(2-methoxyethylacrylate), and/or a nitrocellulose, etc. In some embodiments, the membrane may include a copolymer such as a graft copolymer (for example, poly(propylene-graft-2-methoxyethylacrylate)), e.g., including any one or more of these polymers and/or other suitable polymers. In some cases, the separation membrane may be asymmetric, e.g., having a different separation effectiveness depending on which way blood is passed across the separation membrane to produce plasma. Many such separation membranes may be readily obtained commercially, such as Pall Vivid Plasma Separation Membrane (GF, GX, and GR), as well as other commercially available separation membranes.

During use, blood is moved towards the separation membrane using a suitable driving force to move the blood, for example, vacuum or other reduced pressure as is discussed herein. A fluidic portion of the blood is able to pass across the separation membrane to form plasma or serum on one side of the membrane, while other portions of the blood, e.g., red and white blood cells, are rejected by the membrane and thus form a portion that becomes concentrated in blood cells. For example, serum may be produced if no anticoagulant is present, in accordance with certain embodiments. Either or both portions of the blood may be collected, e.g., in an appropriate storage chamber, for further use, analysis, storage, etc., as is discussed herein.

In some embodiments, a fluid receiving device may include features that are generally directed to substrates for absorbing blood and/or other bodily fluids, for example, a blood spot membrane. Thus, in some embodiments, blood spots may be produced on a blood spot membrane. In these cases, a channel within the device may have a small volume relative to the volume of a blood spot membrane which may be very porous and may collect fluid. The blood spot membrane is used to collect fluid in certain embodiments. The blood spot membrane is not used to separate cells/plasma (as opposed to the separation membranes discussed herein), in certain cases. Fluid may fill all, or a portion of, the blood spot membrane. A second hydrophobic membrane may be positioned on top of the collection membrane in some embodiments. Once fluid contacts the hydrophobic membrane, fluid collection may cease. The blood spot membrane may remain in the device to dry and can then be removed from the device. In some embodiments, the blood spot membrane may be removed from the device and dried outside of the device. In some cases, the membrane is not dried. If a vacuum is used to draw blood towards the blood spot membrane, the vacuum may be released prior to removal of the blood spot membrane from the device, at least in some embodiments.

In one set of embodiments, the substrate is contained within a device for receiving blood from the skin of a subject. Examples of such devices, and details of such devices able to contain a substrate for absorbing blood and/or other bodily fluids, are discussed in detail below.

In one set of embodiments, the substrate for absorbing blood may comprise paper, e.g., that is able to absorb blood or other bodily fluids received by the device. The substrate may be able to partially or wholly absorb any blood (or other bodily fluid) that it comes into contact with. For example, the substrate may comprise filter paper, cellulose filters, cotton-based paper, e.g., made from cellulose filters, cotton fibers (e.g., cotton linters), glass fibers, or the like. Specific non-limiting examples that are commercially available include Schleicher & Schuell 903™ or Whatman 903™ paper (Whatman 903™ Specimen Collection Paper) (Whatman International Limited, Kent, UK), or Ahlstrom 226 specimen collection paper (Ahistrom Filtration LLC, Mount Holly Springs, Pa.). In some embodiments, the paper may be one that is validated in compliance with the requirements of the CLSI (Clinical and Laboratory Standards Institute) LA4-A5 consensus standard. However, other materials may also be used for the substrate for absorbing blood, instead of and/or in addition to paper. For example, the substrate for absorbing blood (or other bodily fluids) may comprise gauze, cloth, cardboard, foam, foamboard, paperboard, a polymer, a gel, or the like. In some cases, the absorbent substrate may have a surface area of at least about 0.001 m²/g, at least about 0.003 m²/g, at least about 0.005 m²/g, at least about 0.01 m²/g, at least about 0.03 m²/g, at least about 0.05 m²/g, at least about 0.1 m²/g, at least about 0.3 m²/g, at least about 0.5 m²/g, or at least about 1 m²/g. In some cases, the absorbent substrate may have a surface area of about 100 g/m² to about 200 g/m², or about 150 g/m² to about 200 g/m².

The blood (or other bodily fluid) may be absorbed into the substrate such that the blood becomes embedded within fibers or other materials forming the substrate, and/or such that the blood becomes embedded in spaces between the fibers or other materials forming the substrate. For example, the blood may be held within or on the substrate mechanically and/or chemically (e.g., via clotting and/or reaction with fibers or other materials forming the substrate).

In some cases, the substrate may absorb a relatively small amount of blood. For example, less than about 1 ml, less than about 800 microliters, less than about 600 microliters, less than about 500 microliters, less than about 400 microliters, less than about 300 microliters, less than about 200 microliters, less than about 100 microliters, less than about 80 microliters, less than about 60 microliters, less than about 40 microliters, less than about 30 microliters, less than about 20 microliters, less than about 10 microliters, or less than about 1 microliter of blood may be absorbed into the substrate.

The substrate may be of any shape or size. In some embodiments, the substrate may be substantially circular, although in other embodiments, other shapes are possible, e.g., square or rectangular. The substrate may have any suitable area. For example, the substrate may be large enough to contain only one spot, of blood (e.g., of the above volumes), or more than one spot in some embodiments. For example, the substrate may have an area of no more than about 1 cm², no more than about 3 cm², no more than about 5 cm², no more than about 7 cm², no more than about 10 cm², no more than about 30 cm², no more than about 50 cm², no more than about 100 cm², no more than about 300 cm², no more than about 500 cm², no more than about 1000 cm², or no more than about 3000 cm².

In some embodiments, a “tab” or a handle, or other separate portion, may be present on or proximate the substrate, e.g., to facilitate analysis and/or manipulation of the absorbed blood or other bodily fluid. The handle may be any portion that can be used to manipulate the substrate. For example, a handle may be used to remove the substrate from the device for subsequent shipping and/or analysis, e.g., without requiring a person to touch the blood spot itself in order to manipulate the substrate. The handle may be formed from the substrate, and/or different material, for example, plastic, cardboard, wood, metal, etc. In some cases, the handle may surround all, or at least a portion of, the substrate.

In certain embodiments, the substrate may include stabilizers or other agents, e.g., for stabilizing and/or treating the blood in the substrate. Non-limiting examples of stabilizers include chelating agents, enzyme inhibitors, or lysing agents. Examples of chelating agents include, but are not limited to, EDTA (ethylenediaminetetraacetic acid) or dimercaprol. Examples of enzyme inhibitors include, but are not limited to, protease inhibitors (e.g., aprotinin, bestatin, calpain inhibitor I and II, chymostatin, E-64, leupeptin or N-acetyl-L-leucyl-L-leucyl-L-argininal, alpha-2-macroglobuline, Pefabloc SC, pepstatin, PMSF or phenylmethanesulfonyl fluoride, TLCK, a trypsin inhibitor, etc.) or reverse transcriptase inhibitors (e.g., zidovudine, didanosine, zalcitabine, stavudine, lamivudine, abacavir, emtricitabine, entecavir, apricitabine, etc.). Non-limiting examples of lysing agents include distilled water or guanidinium thiocyanate.

The following are each also incorporated herein by reference in their entireties: U.S. Pat. Apl. Ser. Nos. 62/842,303; 62/880,137; 62/942,540; 62/948,788; and 62/959,868.

While aspects of the disclosure have been described with reference to various illustrative embodiments, such aspects are not limited to the embodiments described. Thus, it is evident that many alternatives, modifications, and variations of the embodiments described will be apparent to those skilled in the art. Accordingly, embodiments as set forth herein are intended to be illustrative, not limiting. Various changes may be made without departing from the spirit of aspects of the disclosure.

What is claimed is:

1. A device for receiving fluid from a subject, comprising:
a device actuator;
one or more flow activators configured to cause fluid to be released from the subject;
a vacuum source;
a support having a sidewall; and
an interface configured to contact the subject's skin, the interface defining an opening through which fluid is received from the subject,
wherein at least a portion of the interface is moveable relative to the sidewall of the support.

2. The device of claim 1, wherein the interface is made of a first material and the sidewall of the support is made of a second material, the first material having a lower Young's modulus than a Young's modulus of the second material.

3. The device of claim 1 or 2, wherein the interface includes a main body and a first section, the first section being connected to the main body by a region having reduced cross-sectional area as compared to the main body, wherein the region permits the first section to move relative to the main body, and wherein the first section is moveable relative to the sidewall of the support.

4. The device of any one of claims 1-3, wherein a diameter of the opening is smaller than a largest diameter of the sidewall of the support.

5. The device of any one of claims 1-4, wherein the sidewall forms a cylindrical shape.

6. The device of any one of claims 1-5, wherein the sidewall forms a funnel shape.

7. The device of any one of claims 1-6, wherein the interface includes a distal surface configured to contact the subject's skin, and the sidewall includes a distal end, wherein a surface area of the distal surface of the interface is larger than a surface area of the distal end of the sidewall.

8. The device of any one of claims 1-7, wherein the interface is attached to the sidewall.

9. The device of any one of claims 1-8, wherein the interface is made of silicone.

10. The device of any one of claims 1-9, wherein the interface is made of thermoplastic elastomer.

11. The device of any one of claims 1-10, wherein the interface has a horizontal portion and a vertical portion, where the horizontal portion is moveable relative to the support.

12. The device of any one of claims 1-11, wherein the interface has a horizontal portion and a C-shaped portion that transitions the interface from the support to the horizontal portion of the interface.

13. The device of any one of claims 1-12, wherein the interface comprises a horizontal shape with a rounded corner at the opening.

14. The device of any one of claims 1-13, wherein the interface comprises an L-shape having a vertical portion and a horizontal portion.

15. The device of claim 14, wherein a neck portion joins the vertical portion to the horizontal portion, the neck portion having a smaller width than a width of the vertical portion and a width of the horizontal portion.

16. The device of any one of claims 1-15, wherein the vacuum source comprises vacuum bulb that generates a vacuum when the bulb expands from a smaller volume to a larger volume.

17. The device of any one of claims 1-16, further comprising a storage chamber configured to store fluid received into the device.

18. The device of claim 17, wherein the storage chamber is removable from the device.

19. The device of any one of claims 1-18, wherein the flow activators comprise needles.

20. The device of any one of claims 1-19, wherein the flow activators comprise blades.

21. A device for receiving fluid from a subject, comprising:
a housing including an inlet sidewall defining an opening to receive fluid into the housing;
a device actuator;
one or more flow activators configured to cause fluid to be released from the subject; and
an interface configured to contact the subject's skin,
wherein the interface includes a distal surface configured to contact the subject's skin, and the inlet sidewall includes a distal end, wherein a surface area of the distal surface of the inlet sidewall is larger than a surface area of the distal end of the inlet sidewall.

22. The device of claim 21, wherein a portion of the interface extends radially inwardly from the inlet sidewall.

23. The device of claim 22, wherein the interface includes a hole through which fluid is received, wherein a diameter of the hole of the interface is smaller than a diameter of the opening of the inlet sidewall.

24. The device of any one of claims 21-23, wherein the flow activators comprise needles.

25. The device of any one of claims 21-24, wherein the flow activators comprise blades.

26. A device for receiving fluid from a subject, comprising:
a device actuator;
one or more flow activators configured to cause fluid to be released from the subject;
a vacuum source; and
an interface configured to contact the subject's skin, the interface defining an opening through which fluid is received from the subject, the interface having a sidewall comprising a funnel shape.

27. The device of claim 26, further comprising a lubricant on at least a portion of the inlet sidewall.

28. The device of claim 27, wherein the lubricant comprises petroleum jelly.

29. The device of any one of claims 26-28, wherein the flow activators comprise needles.

30. The device of any one of claims 26-29, wherein the flow activators comprise blades.

31. A device for receiving fluid from a subject, comprising:
a device actuator;
one or more flow activators configured to cause fluid to be released from the subject;
a vacuum source comprising a flexible dome made of a first material; and
a shell made of a second material having a higher Young's modulus than that of the first material, the device actuator being moveable relative to the shell,
wherein movement of the device actuator relative to the shell causes compression of the flexible dome.

32. The device of claim 31, wherein the shell includes an opening through which at least a portion of the device actuator extends.

33. The device of claim 32, wherein the device actuator includes a user-contacting portion and a stem, wherein the stem extends through the opening of the shell.

34. The device of any one of claims 31-33, wherein the flexible dome has a first shape prior to compression and a second shape during compression, and the flexible dome is biased to return to its first shape when the flexible dome is no longer subjected to compression.

35. The device of claim 34, wherein return of the flexible dome from the second shape back to the first shape creates vacuum.

36. The device of claim 35, further comprising a one-way vent that permits movement of air through the vent during compression of the flexible dome from the first shape to the second shape, but prevents movement of air through the vent during return of the flexible dome from the second shape back to the first shape.

37. The device of any one of claims 31-36, wherein the flexible dome includes a wall having an indented circumferential shoulder.

38. The device of any one of claims 31-37, further comprising a ratchet mechanism that resists movement of the device actuator relative to the shell in a direction away from the flexible dome.

39. The device of claim 38, wherein the ratchet mechanism comprises a ratchet and pawl.

40. The device of claim 39, wherein the pawl is attached to the shell and the ratchet is attached to the device actuator.

41. The device of claim 40, wherein the device actuator includes a user-contacting portion and a stem, and the ratchet is located on the stem.

42. The device of claim 40 or 41, wherein the shell includes an opening through which at least a portion of the device actuator extends, and the pawl is located in the opening.

43. The device of claims 39-42, wherein the ratchet includes a plurality of teeth and a first tooth, the user-contacting portion being further away from the first tooth than the plurality of teeth, wherein the first tooth is reversed in direction relative to the plurality of teeth.

44. The device of any one of the above claims, further comprising a retraction actuator and a piercing assembly, the piercing assembly including a deployment actuator and the one or more flow activators, the piercing assembly being moveable in a deployment direction toward the opening, the retraction actuator being compressed during movement of the piercing assembly toward the opening.

45. The device of claim 44, wherein the deployment actuator stores potential energy during movement of the piercing assembly toward the opening.

46. The device of claim 44 or 45, wherein the deployment actuator releases stored potential energy upon contact of the piercing assembly with a user's skin, causing the one or more flow activators to move toward and pierce the user's skin.

47. The device of claim 44-46, wherein the retraction actuator comprises a spring having one or more cantilevered helical arms.

48. The device of claims 44-47, wherein the piercing assembly further includes a guide housing, the one or more flow activators being moveable through at least a portion of the guide housing during deployment, wherein the guide housing is in contact with the retraction spring.

49. The device of claim 48, wherein the retraction spring slides against the guide housing during compression of the retraction spring.

50. The device of claim 44, 47, or 49, wherein the retraction actuator is attached to the support.

51. The device of claim 48, wherein the guide housing includes a notch that is in contact with the retraction spring.

52. The device of claims 48-51, wherein the piercing assembly further includes a latch engaged with a support on the guide housing, the latch being moveable through at least a portion of the guide housing when the latch is released.

53. The device of claim 52, further comprising a latch release, wherein movement of the latch release exceeding a threshold travel distance disengages the latch from the support.

54. The device of claim 52 or 53, further comprising a latch release, wherein an actuation force applied to the latch release exceeding a threshold force disengages the latch from the support, independent of travel distance of the latch release.

55. The device of claims 52-54, wherein the guide housing includes latch tracks that guide linear movement of the latch through the guide housing.

56. The device of claims 48-55, wherein the support includes guide housing tracks that guide linear movement of the guide housing relative to the support.

57. The device of claims 44-56, wherein the deployment actuator comprises a deployment spring and the retraction actuator comprises a retraction spring, and wherein a spring constant of the deployment spring is stiffer than a spring constant of the retraction spring.

58. The device of claims 44-57, wherein the deployment actuator comprises a deployment spring and the retraction actuator comprises a retraction spring, and wherein the deployment spring and the retraction spring are arranged in series.

59. The device of claims 44-48, wherein the deployment actuator comprises a deployment spring and the retraction actuator comprises a retraction spring, and wherein the deployment spring and the retraction spring are arranged in parallel.

60. The device of claims 52-59, wherein the piercing assembly further includes a push cap including a contact surface configured to contact the latch to release the latch, the deployment spring being compressed between the push cap and the latch when the push cap moves toward the latch, and wherein actuation of the device causes the push cap to move in a deployment direction toward the opening.

61. The device of claim 60, wherein the guide housing includes push cap tracks that guide linear movement of the push cap through the guide housing.

62. The device of claim 60 or 61, wherein the deployment spring is attached to the push cap.

63. The device of claims 52-62, wherein the deployment actuator comprises a deployment spring, and wherein the latch and the deployment spring are integrally formed as a single component.

64. The device of claims 52-63, further comprising a latch release, wherein the deployment actuator comprises a deployment spring, and wherein the latch release and the deployment spring are integrally formed as a single component.

65. The device of claims 52-63, further comprising a latch release, wherein the deployment actuator comprises a deployment spring, and wherein the latch, latch release and the deployment spring are integrally formed as a single component.

66. The device of claims 44-65, wherein the deployment actuator comprises a spring having an undulating, non-coil shape.

67. The device of any one of claims 48 to 52, wherein the guide housing includes a spring track that receives at least a portion of the spring and guides linear movement of the spring through the guide housing.

68. The device of claims 52-67, wherein the latch has a latch width spanning from a first arm of the latch to a second arm of the latch, and the deployment actuator comprises a spring having an undulating, non-coil shape, the spring having a spring width spanning from a first curve of the spring to a second curve of the spring, the first and second curve facing opposite directions, and wherein the spring width is oriented perpendicular to the latch width.

69. The device of any one of the above claims, further comprising a piercing assembly including a deployment actuator and the one or more flow activators, and further comprising a positive stop that limits movement distance of the one or more flow activators.

70. The device of claim 69, wherein the positive stop comprises a peg that abuts against a contact surface.

71. The device of claim 70, further comprising a guide housing, the one or more flow activators being moveable through at least a portion of the guide housing during deployment, wherein the peg is coupled to the deployment actuator, and the contact surface is coupled to a guide housing.

72. The device of claim 36, wherein the one-way vent comprises an umbrella valve.

73. The device of any one of claims 31-36, wherein the flexible dome is harder to compress during an initial phase of compression than during a final phase of compression.

74. A device comprising:
a flexible dome having a first shape prior to compression and a second shape during compression, the flexible dome being biased to return toward the first shape when the flexible dome is no longer subjected to compression; and
a one-way vent, wherein air exits the flexible dome through the one-way vent as the flexible dome is compressed,
wherein the flexible dome is harder to compress during an initial phase of compression than during a later phase of compression.

75. The device of claim 74, wherein return of the flexible dome from the second shape back toward the first shape creates vacuum.

76. The device of any one of claim 74 or 75, wherein the flexible dome includes a wall having an indented circumferential shoulder.
